(19) United States
(12) Patent Application Publication
(10) Pub. No.: US 2026/0002158 A1
(43) Pub. Date: Jan. 1, 2026

(54) VESICLE-BASED COMPOSITIONS AND USES THEREOF

(71) Applicants: NATIONAL UNIVERSITY OF SINGAPORE; Nanyang Technological University, Singapore (SG); Carmine Therapeutics Pte Ltd, Singapore (SG)

(72) Inventors: Thi Nguyet Minh Le, Singapore (SG); Boya Peng, Singapore (SG); Cao Dai Phung, Singapore (SG); Dahai Luo, Singapore (SG); Wee Yeh Ronne Yeo, Singapore (SG); Mai Trinh Nguyen, Singapore (SG)

(21) Appl. No.: 18/846,192

(22) PCT Filed: Mar. 13, 2023
(86) PCT No.: PCT/SG2023/050161

§ 371 Date: Sep. 11, 2024

(30) Foreign Application Priority Data
Mar. 11, 2022 (SG) 10202202517W

(51) Int. Cl. C12N 15/113; A61K 47/69; A61P 35/00; C12N 15/117

(52) U.S. Cl. CPC C12N 15/1135; A61K 47/6901; A61P 35/00; C12N 15/1138; C12N 15/117; C12N 2310/11; C12N 2310/17; C12N 2320/31; C12N 2320/32

(57) ABSTRACT

Disclosed are vesicle-based compositions and uses thereof. The composition comprising a vesicle, such as a red blood cell-derived extracellular vesicle (RBCEV), for delivery of a retinoic acid inducible gene I receptor (RIG-I) agonist to a cell. Also disclosed are methods for treating diseases by administering the vesicle-based compositions.

### FIELD OF THE INVENTION

The present invention relates generally to the field of molecular biology. In particular, the present invention relates to vesicle-based compositions and the uses thereof.

### BACKGROUND OF THE INVENTION

Many therapies, including RNA oligonucleotides, small interference RNAs (siRNAs) and antisense oligonucleotides, are being developed to target many disease genes that were once considered undruggable. Despite their potential, the clinical application of such therapies continues to be restrained by current inefficient delivery of these molecules to target cells, as well as toxicity that can induce organ damage and cause poor survival.

Lipid-based nanoparticles were developed to deliver, for example, siRNA for cancer treatment, and they were observed to have little toxicity. However, off-target effects were observed when lipid-based nanoparticles were used with the conventional siRNAs, thereby causing an accumulation of the siRNA in the wrong cells and a decrease in effectiveness as an anti-cancer therapy.

There is therefore a need to provide improved compositions addressing at least some of the above problems.

### SUMMARY

In one aspect, the present disclosure refers to a composition comprising a vesicle for delivery of a retinoic acid inducible gene I receptor (RIG-I) agonist to a cell.

In another aspect, the present disclosure refers to a composition comprising:
a red blood cell-derived extracellular vesicle (RBCEV);
an immunomodulatory RNA (immRNA) comprising or consisting of 5′-GGAUUUCCACCUUCGGGGGAAAUCC-3′ (SEQ ID NO: 1), wherein the immunomodulatory RNA (immRNA) further comprises a 5′ triphosphate cap; and/or an antisense oligonucleotide (ASO) comprising or consisting of 5′-GGAAGUUAGGGUCUCAGGCCCUAACUUCC-3′ (SEQ ID NO: 2), wherein the antisense oligonucleotide (ASO) further comprises a 5′ triphosphate cap.

In another aspect, the present disclosure refers to a composition comprising:
a lipid nanoparticle;
an immunomodulatory RNA (immRNA) comprising 5′-GGAUUUCCACCUUCGGGGGAAAUCC-3′ (SEQ ID NO: 1), wherein the immunomodulatory RNA (immRNA) further comprises a 5′ triphosphate cap; and/or
a KRAS-ASO.

In another aspect, the present disclosure refers to a method of delivering the retinoic acid inducible gene I receptor (RIG-I) agonist into a cell, comprising administering a pharmaceutically effective amount of the composition as disclosed herein, the pharmaceutical composition as disclosed herein, or the kit as disclosed herein to the cell.

In another aspect, the present disclosure refers to a method of treating a disease comprising administering a pharmaceutically effective amount of the composition as disclosed herein, the pharmaceutical composition as disclosed herein, or the kit as disclosed herein to a subject in need thereof.

### BRIEF DESCRIPTION OF THE DRAWINGS

The invention will be better understood with reference to the detailed description when considered in conjunction with the non-limiting examples and the accompanying drawings, in which:

FIG. 1A is a schematic showing fluorescence-activated cell sorting (FACS) analysis of RBCEVs loaded with FAM-labelled antisense oligonucleotide (ASO) using electroporation, Exo-Fect and REG1. As a negative control, red blood cells-derived extracellular vesicles (RBCEVs) were incubated with FAM-ASO without electroporation or loading reagents.

FIG. 1B is a schematic showing the average mean intensity of FAM in RBCEVs loaded with FAM-labelled ASO using electroporation, Exo-Fect and REG1 (n=3), compared to the extracellular vesicles (EVs) only condition for statistical analysis.

FIG. 1C is a schematic showing transmission electron microscopy images of FAM-ASO-loaded RBCEVs. Scale bar, 200 nm.

FIG. 1D is a schematic showing the size distribution of FAM-ASO-loaded RBCEVs, determined using ZetaView® nanoparticle tracking analyser.

FIG. 1E is a schematic showing representative immunofluorescent images of CA1a cells taking up FAM-ASO in RBCEVs. Nuclei were stained with Hoechst (blue). Plasma membranes were stained with CellMask Deep Red (red). Scale bar, 20 μm.

FIG. 1F is a schematic showing the average mean intensity of FAM in CA1a cells as in (E), compared to untreated control for statistical analysis.

FIG. 1G is a schematic showing FACS analysis of CA1a cells taking up FAM-ASO-loaded RBCEVs (n=3), compared to untreated control for statistical analysis.

FIG. 1H is a schematic showing the viability of human breast cancer CA1a cells treated with 0.05 μg/μl NC-ASO loaded RBCEVs at different time points. Viable cells were quantified by normalizing the absorbance readings to the untreated control at each time point (n=3).

FIG. 1I is a schematic showing the loading efficiency of miR-125b ASO in RBCEVs using Exo-Fect and REG1, determined using gel electrophoresis (n=3).

FIG. 1J is a schematic showing quantitative polymerase chain reaction (qPCR) analysis of miR-125b fold change (FC) relative to EVs only condition, normalized to U6B in CA1a cells treated with different doses of RBCEVs containing miR-125b ASO (n=3). All bar graphs represent mean±SEM. Ns, not significant; *p<0.05, **p<0.01, ***p<0.001, and ****p<0.0001 determined by Student's two-tailed t-test. FIG. 1 shows that RBCEVs can be loaded with small RNAs using multiple methods for the delivery of RNAs to cancer cells.

FIG. 2A is a schematic showing the design of immunomodulatory RNA (immRNA) with 5′ triphosphate (ppp).

FIG. 2B-D is a schematic showing qPCR analysis of RIG-I encoding mRNA (Ddx58) and its downstream effectors relative to Gapdh in mouse breast cancer 4T1 cells (B), human breast cancer CA1a cells (C) and human lung cancer H358 cells (D) treated with 0.1 μg/μL unloaded RBCEVs, NC-RNA-loaded RBCEVs and immRNA loaded RBCEVs for 24 hours (n=4, RNA loaded using REG1).

FIG. 2E is a schematic showing the average luciferase activity in A549-Dual™ and A549-Dual™ RIG-I^{-/-} cells treated with PBS, 0.05 μg/μL unloaded RBCEVs, NC-RNA-loaded RBCEVs and immRNA-loaded RBCEVs for 24 and 48 hours (n=5-9).

FIG. 2F is a schematic showing multiplex immunoassay analysis of cytokines in the conditioned media of 4T1 cells treated with 0.1 μg/μL unloaded RBCEVs, NC-RNA-loaded RBCEVs and immRNA-loaded RBCEVs for 48 hours (n=3).

FIG. 2G to I is a schematic showing FACS analysis of the average percentage of ANXV+PI+ population in 4T1 cells (G), CA1a cells (H) and H358 cells (I) after a treatment with 0.1 μg/μL unloaded RBCEVs, NC-RNA-loaded RBCEVs and immRNA-loaded RBCEVs for 72 hours (n=4). All bar graphs represent mean±SEM. *P<0.05, **P<0.01, ***P<0.001 and ****P<0.0001 determined by Student's two-tailed t-test. FIG. 2 shows that RBCEVs deliver immunomodulatory RNA to activate the RIG-I pathway and induce immunogenic cell death in cancer cells.

FIG. 3A is a schematic showing miR-125b sequence and the design of ASO against miR-125b with and without 5′ triphosphate. The seed sequence of miR-125b is coloured in red and underlined. A triphosphate group (PPP) was added to the 5′ end of 3p-125b-ASO whereas the first four nucleotides were replaced with GG. A short sequence (grey circles) was added to the 3′ end of 3p-125b-ASO during IVT.

FIG. 3B is a schematic showing qPCR analysis of miR-125b relative to snoRNA234 in 4T1 cells treated with 0.1 μg/μL unloaded or NC-RNA-loaded, 125b-ASO-loaded and 3p-125b-ASO-loaded RBCEVs for 24 hours (n=4, RNA loaded using REG1).

FIG. 3C is a schematic showing qPCR analysis of Ddx58 and its downstream effectors relative to Gapdh in 4T1 cells treated with RBCEVs as in (B).

FIG. 3D is a schematic showing qPCR analysis of miR-125b relative to U6B in CA1a cells treated with RBCEVs as in (B).

FIG. 3E is a schematic showing qPCR analysis of DDX58 and its downstream effectors relative to GAPDH in CA1a cells treated with RBCEVs as in (B).

FIG. 3F is a schematic showing qPCR analysis of miR-125b relative to U6B in H358 cells treated with RBCEVs as in (B).

FIG. 3G is a schematic showing qPCR analysis of DDX58 and its downstream effectors relative to GAPDH in H358 cells treated with RBCEVs as in (B).

FIG. 3H is a schematic showing the average luciferase activity in A549-Dual™ and A549-Dual™ RIG-I^{-/-} cells treated with PBS, 0.05 μg/μL unloaded RBCEVs, NC-RNA-loaded RBCEVs and 3p-125b-ASO-loaded RBCEVs for 24 and 48 hours (n=5-8).

FIG. 3I is a schematic showing multiplex immunoassay analysis of cytokines in the conditioned media of 4T1 cells treated with 0.1 μg/μL unloaded, NC-RNA-loaded and 3p-125b-ASOloaded RBCEVs for 48 hours (n=3).

FIG. 3J to L is a schematic showing FACS analysis revealing the average percentage of ANXV+PI+ population in 4T1 cells (J), CA1a cells (K) and H358 cells (L) after a treatment with 0.1 μg/μL unloaded, NC-RNA-loaded, 125b-ASOloaded and 3p-125b-ASO-loaded RBCEVs for 72 hours (n=4). All bar graphs represent mean±SEM. **P<0.01, ***P<0.001 and ****P<0.0001 determined by Student's two-tailed t-test. FIG. 3 shows that RBCEVs deliver bi-functional ASOs to simultaneously inhibit oncogenic miR-125b and activate the RIG-I pathway leading to cell death in cancer cells.

FIG. 4A is a schematic showing the treatment of mouse 4T1 mammary tumours in BALB/c mice with intratumoral delivery of immRNA in RBCEVs.

FIG. 4B is a schematic showing the volume of 4T1 tumours injected intratumorally with 2.5 mg/kg RBCEVs containing immRNA or NC RNA every three days (n=6 mice).

FIG. 4C is a schematic showing qPCR analysis of the RIG-I pathway gene expression relative to Gapdh in untreated and treated 4T1 tumours (n=6 mice).

FIG. 4D is a schematic showing flow cytometry analysis of immune cells in untreated and treated 4T1 tumours (n=6 mice), presented as the average percentage of each subset in total cells. NEUT, neutrophils; NK, natural killer cells; MP, macrophages; DCs, dendritic cells.

FIG. 4E is a schematic showing ELISA quantification of IFNβ in the sera of mice with 4T1 tumours (n=4 mice).

FIG. 4F is a schematic showing qPCR analysis of cytokine gene expression relative to Gapdh in untreated and treated 4T1 tumours (n=4 mice).

FIG. 4G is a schematic showing representative images of TUNEL staining (orange fluorescence) of untreated and treated 4T1 tumour sections. Cancer-associated fibroblasts (green) were stained with anti-αSMA antibody. Nuclei were stained with Hoechst (blue). Scale bar, 50 μm. T, Tumour; S, Stroma.

FIG. 4H is a schematic showing the average mean intensity per nucleus area of TUNEL staining signals (n=4 mice).

FIG. 4I is a schematic showing the treatment for human CA1a mammary tumours in NSG-SGM3 mice with intratumoral delivery of immRNA-loaded RBCEVs.

FIG. 4J is a schematic showing the volume of CA1a tumours injected intratumorally with 2.5 mg/kg RBCEVs loaded with immRNA or NC RNA every three days (n=6 mice).

FIG. 4K is a schematic showing qPCR analysis of RIG-I pathway gene expression relative to GAPDH in untreated and treated CA1a tumours (n=6 mice).

FIG. 4L is a schematic showing flow cytometry analysis of immune cells in untreated and treated CA1a tumours (n=6 mice). All bar graphs represent mean±SEM. *P<0.05, **P<0.01, ***P<0.001 and ****P<0.0001 determined by Student's two-tailed t-test. FIG. 4 shows that intratumoral delivery of immRNA suppresses breast cancer growth by triggering RIG-I mediated immune responses.

FIG. 5A is a schematic showing treatment of mouse 4T1 mammary tumours in BALB/c mice with intratumoral delivery of 3p-125b-ASO in RBCEVs.

FIG. 5B is a schematic showing the volume of 4T1 tumours injected intratumorally with 5 mg/kg RBCEVs containing 3p-125b-ASO or NC RNA every three days (n=6 mice).

FIG. 5C is a schematic showing qPCR analysis of miR-125b relative to sno234 in untreated and treated 4T1 tumours (n=6 mice).

FIG. 5D is a schematic showing qPCR analysis of RIG-I pathway gene expression relative to Gapdh in untreated and treated 4T1 tumours (n=6 mice).

FIG. 5E is a schematic showing flow cytometry analysis of immune cells in untreated and treated 4T1 tumours (n=5 mice). NEUT, neutrophils; NK, natural killer cells; MΦ, macrophages; DCs, dendritic cells.

FIG. 5F is a schematic showing multiplex immunoassay analysis of cytokine concentration (conc.) in the tumour lysates of mice (n=3 mice).

FIG. 5G is a schematic showing qPCR analysis of cytokine gene expression relative to Gapdh in untreated and treated 4T1 tumours (n=5 mice).

FIG. 5H is a schematic showing representative images of TUNEL staining (orange fluorescence) of untreated and treated 4T1 tumour sections. Cancer-associated fibroblasts (green) were stained with anti-αSMA antibody. Nuclei were stained with Hoechst (blue). Scale bar, 50 μm. T, Tumour; S, Stroma.

FIG. 5I is a schematic showing the average mean intensity per nucleus area of TUNEL staining signals (n=4 mice). All bar graphs represent mean±SEM. *P<0.05, **P<0.01, ***P<0.001 and ****P<0.0001 determined by Student's two-tailed t-test. FIG. 5 shows that intratumoral delivery of bi-functional ASO suppresses breast cancer growth by triggering apoptosis and RIG-I mediated immune responses

FIG. 6A is a schematic showing RBCEV modification: RBCEVs were conjugated with an anti-EGFR nanobody and loaded with immRNA.

FIG. 6B is a schematic showing FACS analysis of 4T1-hEGFR cells and parental 4T1 cells treated with 0.02 μg/μL uncoated, control (Ctrl)-VHH-coated and EGFR-VHH-coated CFSE-labeled RBCEVs (n=3).

FIG. 6C is a schematic showing FACS analysis of 4T1-hEGFR cells and parental 4T1 cells treated with 0.02 μg/μL uncoated, Ctrl-VHH-coated and EGFR-VHH-coated FAM-NC-ASO-loaded RBCEVs (n=3).

FIG. 6D is a schematic showing qPCR analysis of the RIG-I pathway gene expression relative to Gapdh in 4T1-hEGFR cells and parental 4T1 cells treated with 0.1 μg/μL uncoated, Ctrl-VHH-coated and EGFR-VHH-coated RBCEVs containing immRNA (n=3).

FIG. 6E is a schematic showing intrapulmonary delivery of EGFR-targeted CFSE-RBCEVs in mice bearing metastatic 4T1 breast cancer.

FIG. 6F is a schematic showing FACS analysis of CFSE signals in tumour cells and immune cells in the lungs of mice treated with 25 mg/kg uncoated, Ctrl-VHH-coated and EGFR-VHH-coated CFSE-RBCEVs (n=3 mice). NEUT, neutrophils; MΦ, macrophages; DCs, dendritic cells. All bar graphs represent mean±SEM. **P<0.01 and ***P<0.001 determined by Student's two-tailed t-test. FIG. 6 shows that conjugation of RBCEVs with EGFR-binding nanobody promotes specific delivery of immRNA to metastatic breast cancer cells.

FIG. 7A is a schematic showing intrapulmonary treatments for mice bearing breast cancer metastasis.

FIG. 7B is a schematic showing flow cytometry analysis of tumour burden in the lungs using anti-human-EGFR antibody after five treatments.

FIG. 7C is a schematic showing the average percentage of hEGFR-positive tumour cells in the lungs as shown in (B) (n=4 mice).

FIG. 7D is a schematic showing average percentage of metastatic area in the lungs treated with 25 mg/kg uncoated, Ctrl-VHH-coated and EGFR-VHH-coated RBCEVs containing immRNA after five treatments (n=4 mice).

FIG. 7E is a schematic showing representative hematoxylin and eosin (H & E)-stained lung sections from mice as in (D). Scale bar, 50 μm.

FIG. 7F is a schematic showing qPCR analysis of the RIG-I pathway gene expression relative to Gapdh in the lungs of mice as in (A) (n=4 mice).

FIG. 7G is a schematic showing FACS analysis of immune cells in the lungs of mice as in (A) (n=4 mice).

FIG. 7H is a schematic showing representative images of TUNEL staining (orange fluorescence) of lung sections from mice as in (A). Nuclei were stained with Hoechst (blue). Scale bar, 20 μm.

FIG. 7I is a schematic showing average mean intensity per nucleus area of TUNEL staining signals (n=4 mice).

FIG. 7J is a schematic showing multiplex immunoassay of cytokines in the lung homogenates of mice with 4T1-hEGFR metastatic tumours (n=3 mice). All bar graphs represent mean±SEM. *P<0.05, **P<0.01, ***P<0.001 and ****P<0.0001 determined by Student's two-tailed t-test. FIG. 7 shows that intrapulmonary delivery of immRNA using EGFR-targeted RBCEVs suppresses breast cancer metastasis in the lung

FIG. 8A is a schematic showing flow cytometry analysis of activation and polarization of DCs and macrophages in the lungs of mice as in FIG. 7a (n=3-4 mice). MΦ, macrophages.

FIG. 8B is a schematic showing the average percentage of cells as shown in (A) (n=3-4 mice).

FIG. 8C is a schematic showing flow cytometry analysis of CD69 and granzyme B in CD8+ tumour-infiltrating lymphocytes (TILs) from the lungs of mice as in FIG. 7a (n=3-4 mice).

FIG. 8D is a schematic showing the average percentage of CD69 and granzyme B in CD8+ TILs as shown in (C).

FIG. 8E is a schematic showing ELISA quantification of IFNγ from tumour-specific CD8+ TILs in the culture supernatants at 48 h (n=3-4 mice).

FIG. 8F is a schematic showing tumour-specific cytotoxic T lymphocyte (CTL) activity of CD8+ TILs (n=3-4 mice). All bar graphs represent mean±SEM. ns, not significant; *p<0.05, **p<0.01, ***p<0.001, and ****p<0.0001 determined by Student's two-tailed t-test. FIG. 8 shows that EGFR-targeted immRNA-loaded RBCEVs induce DC activation, promote M1 macrophage polarization and potentiate tumour-specific CD8+ T cell activity.

FIG. 9A is a schematic showing the design and chemical modification of ASOs targeting KRAS G12D and G12V.

FIG. 9B is a schematic showing the knockdown of KRAS in A427 and AsPC-1 cells bearing KRAS G12D mutant and H1975 and DU145 cells bearing wild-type KRAS after treatment with RBCEVs loaded with KRAS G12D ASOs as determined by Western Blot (n=3).

FIG. 9C is a schematic showing the knockdown of KRAS in A427 cells bearing KRAS G12D mutant and H1975 and DU145 cells bearing wild-type KRAS after treatment with RBCEVs loaded with KRAS G12D ASOs as determined by qPCR analysis (n=3).

FIG. 9D is a schematic showing the knockdown of KRAS in AsPC-1 cells bearing KRAS G12D mutant and H1975 and DU145 cells bearing wild-type KRAS after treatment with RBCEVs loaded with KRAS G12D ASOs as determined by qPCR analysis (n=3).

FIG. 9E is a schematic showing the cell viability changes caused by RBCEVs loaded with KRAS G12D ASO2 in A427 cells determined by CCK-8 assay (n=5).

FIG. 9F is a schematic showing the cell viability changes caused by RBCEVs loaded with KRAS G12D ASO2 in AsPC-1 cells determined by CCK-8 assay (n=5).

FIG. 9G is a schematic showing the knockdown of KRAS in H441 cells bearing KRAS G12V mutant and H1975 cells bearing wild-type KRAS after treatment with RBCEVs loaded with KRAS G12V ASOs as determined by Western Blot (n=3).

FIG. 9H is a schematic showing the knockdown of KRAS in H441 cells bearing KRAS G12V mutant and H1975 cells bearing wild-type KRAS after treatment with RBCEVs loaded with KRAS G12V ASOs as determined by qPCR analysis (n=3).

FIG. 9I is a schematic showing the reduction in cell viability caused by RBCEVs loaded with KRAS G12V ASO2 in A427 cells and H1975 cells determined by CCK-8 assay (n=5). All bar graphs represent mean±SD. ns, *p<0.05, **p<0.01, ***p<0.001, and ****p<0.0001 determined by Student's two-tailed t-test.

FIG. 10 is a schematic showing the delivery of RIG-I agonists using RBCEVs for anti-cancer immunotherapy.

FIG. 11A is a schematic showing a western blot of ALIX, TSG101, GPA, HBA, β-actin and GAPDH in RBCs and RBCEVs.

FIG. 11B is a schematic showing single-EV FACS analysis of GPA in RBCEVs. Gating strategy used to obtain a distinct population of RBCEVs from the background noise.

FIG. 11C is a schematic showing flow cytometry analysis of EpCAM expression in mouse lung epithelial cells and α-EGFR-VHH binding affinity to multiple cells. FIG. 11 shows the characterization of RBCEVs, mouse lung epithelial cells and α-EGFR-VHH binding affinity.

FIG. 12A to D is a schematic showing qPCR analysis of DDX58 and its downstream effectors relative to GAPDH in MDA-MB-468 (A), MDA-MB-231 (B), MCF10A (C) and mouse lung epithelial cells (D) treated with 0.1 μg/μL unloaded or NC-RNA-loaded, immRNA-loaded RBCEVs for 24 hours (n=3-4, RNA loaded using REG1) ND, not detected.

FIG. 12E is a schematic showing representative flow cytometric plots of ANXV/PI staining in 4T1, CA1a, H358, MDA-MB-468 (MB468), MDA-MB-231 (MB231), MCF10A and mouse lung epithelial (mLE) cells treated with 0.1 μg/μL unloaded RBCEVs, NC RNA-loaded RBCEVs and immRNA-loaded RBCEVs for 72 hours.

FIG. 12F is a schematic showing the average percentage of ANXV+PI+ population in MB468, MB231, MCF10A and mLE cells treated with RBCEVs as in (E) (n=3). All bar graphs represent mean±SEM. *P<0.05, **P<0.01 and ****P<0.0001 determined by Student's two-tailed t-test. FIG. 12 shows the effects of immRNA-loaded RBCEVs on cancer cells and non-malignant cells.

FIG. 13A is a schematic showing the secondary structure of 3p-125b-ASO.

FIG. 13B to D is a schematic showing qPCR analysis of DDX58 and its downstream effectors relative to GAPDH in MDA-MB-231 (B), MCF10A (C) and mouse lung epithelial cells (D) treated with 0.1 μg/μL unloaded or NCRNA-loaded, 125b-ASO-loaded and 3p-125b-ASO-loaded RBCEVs for 24 hours (n=3-4, RNA loaded using REG1) ND, not detected.

FIG. 13E is a schematic showing representative flow cytometric plots of ANXV/PI staining in 4T1, CA1a, H358, MDA-MB-231 (MB231), MCF10A and mouse lung epithelial (mLE) cells treated with 0.1 μg/μL unloaded or NC RNA-loaded, 125b-ASO-loaded and 3p-125b-ASO-loaded RBCEVs for 72 hours.

FIG. 13F is a schematic showing the average percentage of ANXV+PI+ population in MB231, MCF10A and mLE cells treated with RBCEVs as in (E) (n=3).

FIG. 13G is a schematic showing representative flow cytometric plots of ANXV/PI staining in 4T1 cells and CA1a cells after a treatment with combined 3p-125-ASO- and immRNA-loaded RBCEVs for 72 hours.

FIG. 13H is a schematic showing qPCR analysis of RIG-I pathway gene expression in 4T1 cells and CA1a cells after a treatment with combined 3p-125-ASO- and immRNA-loaded RBCEVs for 24 hours. All bar graphs represent mean±SEM. *P<0.05, **P<0.01, ***P<0.001 and ****P<0.0001 determined by Student's two-tailed t-test. FIG. 13 shows the effects of 3p-125b-ASO-loaded RBCEVs on cancer cells and non-malignant cells.

FIG. 14A is a schematic showing flow cytometry analysis of immune cells in 4T1 mammary tumours treated with immRNA-loaded RBCEVs as in FIG. 4. Cells were gated based on FSC-A and SSC-A to exclude the debris and dead cells. The single cells were further gated based on FSC-W and FSC-H to exclude doublets and aggregates. The live cells were gated from singlet population base on Sytox™ Blue negative. Subsequently, the fluorescence of phenotypic markers for immune cells were gated based on the fluorescent channels and subjected to viSNE analysis.

FIG. 14B is a schematic showing survival of mice with orthotopic 4T1 tumours treated with 2.5 mg/kg NC-RNA-RBCEVs (i.t.), 2.5 mg/kg immRNA-RBCEVs (i.t.), 2 mg/kg anti-PD-L1 antibody (intraperitoneal (i.p.)) as well as 2.5 mg/kg immRNA-RBCEVs (i.t.) in combination with 2 mg/kg anti-PD-L1 antibody (i.p.) (n=2-4 mice).

FIG. 14C is a schematic showing tumour growth of mice treated as in (B) (n=2-4 mice).

FIG. 14D is a schematic showing ELISA quantification of IFNβ in the sera of mice bearing 4T1 tumours treated as in (B) (n=2-4 mice).

FIG. 14E is a schematic showing representative viSNE plots of immune cells in CA1a mammary tumours treated with immRNAloaded RBCEVs as in FIG. 4.

FIG. 14F is a schematic showing volume of MDA-MB-468 tumours injected intratumorally with 2.5 mg/kg RBCEVs containing immRNA or NC RNA every three days (n=3-4 mice).

FIG. 14G is a schematic showing qPCR analysis of the RIG-I pathway gene expression relative to GAPDH in untreated and treated MDA-MB-468 tumours (n=3-4 mice). All bar graphs represent mean±SEM. **P<0.01, ***P<0.001 and ****P<0.0001 determined by Student's two-tailed t-test. FIG. 14 shows the therapeutic efficacy of immRNA-loaded RBCEVs in mammary tumours.

FIG. 15 is a schematic showing flow cytometry analysis by representative viSNE plots of immune cells in 4T1 mammary tumours treated with 3p-125b-ASO-loaded RBCEVs.

FIG. 16A is a schematic showing qPCR analysis of the RIG-I pathway gene expression relative to Gapdh in 4T1-hEGFR cells and parental 4T1 cells treated with 0.1 μg/μL uncoated, Ctrl-VHH-coated and EGFR-VHH-coated RBCEVs containing immRNA (n=3). All bar graphs represent mean±SEM. **P<0.01 and ***P<0.001 determined by Student's two-tailed t-test.

FIG. 16B is a schematic showing representative viSNE plots of biodistribution of EGFR-VHH-CFSE-RBCEVs in respective cell type in the lungs with 4T1-hEGFR metastatic tumours. FIG. 16 shows EGFR-VHH-RBCEVs target 4T1-hEGFR cells in vitro and 4T1-hEGFR lung metastatic tumours in vivo.

FIG. 17A is a schematic showing representative viSNE plots of respective cell type in the lungs with 4T1 metastatic tumours treated with EGFR-VHH-immRNA-RBCEVs.

FIG. 17B is a schematic showing qPCR analysis of cytokine gene expression relative to Gapdh in the lungs of mice with 4T1-hEGFR metastatic tumours (n=4 mice). All bar graphs represent mean±SEM. *P<0.05, **P<0.01, ***P<0.001 and ****P<0.0001 determined by Student's two-tailed t-test. FIG. 17 shows the flow cytometry analysis of immune cells in the lungs with 4T1 metastatic tumours treated with EGFRVHH-immRNA-RBCEVs.

FIG. 18A is a schematic showing the design and chemical modification of ASOs targeting EGFR L858R and T790M.

FIG. 18B is a schematic showing the knockdown of EGFR in H1975 cells bearing EGFR L858R/T790M mutant and A549 cells bearing wild-type EGFR after treatment with RBCEVs loaded with EGFR L858R ASOs, as determined by Western Blot (n=3).

FIG. 18C is a schematic showing the knockdown of EGFR in H1975 cells bearing EGFR L858R/T790M mutant and A549 cells bearing wild-type EGFR after treatment with RBCEVs loaded with EGFR L858R ASOs, as determined by qPCR analysis (n=3).

FIG. 18D is a schematic showing the reduction in cell viability caused by RBCEVs loaded with EGFR L858R ASO4 in H1975 cells and A549 cells as determined by CCK-8 assay (n=5).

FIG. 18E is a schematic showing the knockdown of EGFR in H1975 cells bearing EGFR L858R/T790M mutant and A549 cells bearing wild-type EGFR after treatment with RBCEVs loaded with EGFR T790M ASOs as determined by Western Blot (n=3).

FIG. 18F is a schematic showing the showing the knockdown of EGFR in H1975 cells bearing EGFR L858R/T790M mutant and A549 cells bearing wild-type EGFR after treatment with RBCEVs loaded with EGFR T790M ASOs as determined by qPCR analysis (n=3).

FIG. 18G is a schematic showing the reduction in cell viability caused by RBCEVs loaded with EGFR T790M ASO4 in H1975 cells and A549 cells determined by CCK-8 assay (n=5). All bar graphs represent mean±SD. ns, *p<0.05, **p<0.01, ***p<0.001, and ****p<0.0001 determined by Student's two-tailed t-test.

FIG. 19A is a schematic showing qPCR analysis of DDX58 and IFN-β expression in A427 cells after treatments with different RBCEV formulations.

FIG. 19B is a schematic showing qPCR analysis of DDX58 and IFN-β expression in CT26 cells after treatments with different RBCEV formulations

FIG. 19C is a schematic showing qPCR analysis of DDX58 and IFN-β expression in AsPC-1 cells after treatments with different RBCEV formulations

FIG. 19D is a schematic showing qPCR analysis of DDX58 and IFN-β expression in H1975 cells after treatments with different RBCEV formulations

FIG. 19E is a schematic showing the cell viability of A427 cells after treatments with different RBCEV formulations, determined by CCK-8 assay.

FIG. 19F is a schematic showing the cell viability of CT26 cells after treatments with different RBCEV formulations, determined by CCK-8 assay.

FIG. 19G is a schematic showing the cell viability of AsPC-1 cells after treatments with different RBCEV formulations, determined by CCK-8 assay.

FIG. 19H is a schematic showing the cell viability of H1975 cells after treatments with different RBCEV formulations, determined by CCK-8 assay.

FIG. 19I is a schematic showing the expression of DDX58 and IFN-β in AsPC-1 cells after treatments with different LNP formulations determined by qPCR.

FIG. 19J is a schematic showing the cell viability of AsPC-1 cells after treatments with different RBCEV formulations determined by CCK-8 assay.

FIG. 19K is a schematic showing the schematic for analysis of THP-1 monocytic cell activation after being incubated with cancer cell culture supernatants.

FIG. 19L is a schematic showing the flow cytometric analysis of CD86 expression in THP-1 cells after being incubated with pretreated pancreatic AsPC-1 cancer cells. All bar graphs represent mean±SD. *p<0.05, **p<0.01, ***p<0.001, and ****p<0.0001 determined by One-Way ANOVA test.

FIG. 20A is a schematic showing the treatment of mCherry-Luciferase-expressing H1975 lung tumor in NSGS mice with intratracheal administration of NC ASO or EGFR L858R ASO in RBCEVs.

FIG. 20B is a schematic showing the tumor progression in mice quantified using the average bioluminescent signals following treatment with EV-NC ASO or EV-L858R ASO over time. The graphs present the mean±SEM (n=5). **p<0.01 determined by two-way ANOVA test.

FIG. 20C is a schematic showing the representative bioluminescent imaging of xenografted mice treated with EV-NC ASO or EV-L858R ASO over time.

FIG. 20D is a schematic showing the representative IHC images of EGFR expression in tumor sections from treated mice at the end of study. Scale bar, 100 μm.

FIG. 21A is a schematic showing the tumor volume in BALB/c mice injected with CT26 cells and treated with RBCEVs that were unloaded or loaded with KRAS-G12D ASO and/or immRNA by intratumoral injections every 2 days. The graphs represent the mean±SEM (n=3). *P<0.05, **P<0.01, determined by Two-way ANOVA test.

FIG. 21B is a schematic showing the tumor weight of RBCEV treated mice at the end point. The graphs present the mean±SEM (n=3). *P<0.05, determined by Student's two-tailed t-test.

FIG. 21C is a schematic showing the representative H&E staining of tumors collected from treated mice. Scale bar, 100 μm.

FIG. 21D is a schematic showing the representative TUNEL staining of tumor sections from treated mice at the end of study. Scale bar, 100 μm.

### DEFINITIONS

As used herein, the term “lipid” refers to a class of organic compounds that comprises carbon, hydrogen, and oxygen atoms. Examples of lipids can be, but are not limited to, eicosanoids, monoglycerides, diglycerides, triglycerides, phospholipids, sphingolipids, terpenes, prenols, fatty acids, waxes, steroids, or phospholipids. Further examples of lipids can be, but are not limited to, cationic lipids, ionizable lipids, PEG-lipids, phosphatidylcholine, phosphatidylethanolamine, or cholesterol.

As used herein, the term “cationic lipid” refers to a lipid which comprises a hydrophilic headgroup, and a hydrophobic tail, wherein a linker which can be but are not limited to, an ether, ester, or amide, links the hydrophilic headgroup and the hydrophobic tail.

As used herein, the term “ionizable lipid” refers to a lipid which is derived from a cationic lipid, wherein the quaternary ammonium head of cationic lipids is substituted with a titratable moiety, and are thus neutral at physiological pH, but positively charged at low pH.

As used herein, the terms “PEG-lipid” and “PEG-modified lipid” are used interchangeably and refer to a lipid which is attached to a polyethylene glycol (PEG), wherein one end of the PEG chain is attached to the hydrophobic tail of the lipid through a linker. The molecular weight of the PEG linked to a lipid can be, but is not limited to, 750, 2000, 5000, or 8000. PEG can be linked to a lipid, such as a lipid of a lipid vesicle, or a lipid nanoparticle, to prolong the blood circulation time of the said lipid vesicle.

As used herein, the term “phospholipid” refers to a lipid which comprises a glycerol backbone, fatty acid tails, and a phosphate group, wherein the phosphate group can be modified with organic molecules which can be, but are not limited to, choline, ethanolamine, or serine. Examples of phospholipids can be, but are not limited to, phosphatidylcholine, phosphatidylethanolamine, phosphatidylinositol, or phosphatidylserine.

As used herein, the term “steroid” refers to a lipid characterized by four carbon rings. Examples of steroids can be, but are not limited to, corticosteroids, progestogens, androgens, estrogens, cholesterol, phytosterols, or ergosterols.

As used herein, the term “vesicle” refers to a structure comprising a lipid layer enclosing liquid or cytoplasm. Vesicles can have either be made of a single lipid layer or a lipid bilayer. Vesicles can be naturally-occurring or can be prepared artificially. Examples of vesicles can be, but are not limited to, liposomes, lysosomes, transport vesicles, secretory vesicles, or extracellular vesicles. In the present disclosure, in one example, vesicles comprise or encapsulate the retinoic acid inducible gene I receptor (RIG-I) agonist which is to be delivered to the cells as defined herein.

As used herein, the terms “extracellular vesicle” or “EV” refer to a cell-derived membrane vesicle that is secreted by cells into the extracellular space. The extracellular vesicle plays an essential role in intercellular communication between cells, wherein the content of the extracellular vesicle can comprise specific molecules that could serve as biomarkers, or function as mediators of different physiological processes. Extracellular vesicles can be derived from different cells, for example, but not limited to red blood cells, cancer cells, fibroblasts, epithelial cells, endothelial cells, immune cells, or platelets. Thus, examples of extracellular vesicles can be, but are not limited to, red blood cell-derived extracellular vesicle, milk-derived extracellular vesicle, plasmid-derived extracellular vesicle, or cancer-cell derived extracellular vesicle. In one example, these extracellular vesicles are to be used to deliver the retinoic acid inducible gene I receptor (RIG-I) agonist to the cells as described herein.

As used herein, the terms “red blood cell-derived extracellular vesicle” or “RBCEV” refer to an extracellular vesicle that is derived from a red blood cell, and are about 120-200 nm in diameter. RBCEVs can be purified, and purified RBCEVs can have an increase in markers that can be, but are not limited to, hemoglobin A (HBA), ALIX, or TSG101m. Purified RBCEVs can have a reduction in markers which can be, but are not limited to, cytoskeleton protein β-actin. RBCEVs can be derived from any red blood cell. When derived from a human red blood cell, RBCEVs can have common characteristics which can be, but are not limited to, a lipid membrane, a size of 100-300 nm, an increase in glycophorin A (GPA), or an increase in intraluminal hemoglobin.

As used herein, the terms “lipid nanoparticle” or “LNP” refer to a spherical vesicle made of lipids, which are positively charged at low pH and neutral at physiological pH. A lipid nanoparticle can comprise for example, but is not limited to, one or more cationic lipids, non-cationic lipids, and/or PEG-modified lipids. In one example, the lipid nanoparticle comprises PEG2000-modified lipid. In another example, LNPs are to be used to deliver the retinoic acid inducible gene I receptor (RIG-I) agonist to the cells as described herein.

As used herein, the term “retinoic acid inducible gene I receptor (RIG-I)” refers to a protein that functions as a cytosolic RNA sensor, and recognises RNA sequences with a 5′ triphosphate moiety, and binds to short double-stranded RNAs with higher affinity.

As used herein, the term “antagonist” refers to a chemical that binds to a target receptor to inhibit downstream effector mechanisms. Inhibition of downstream effector mechanisms by an antagonist may be reversible or irreversible.

As used herein, the term “agonist” refers to a chemical that activates a target receptor to activate downstream effector mechanisms to produce a biological response. An agonist can either be endogenous or exogenous. In one example described herein the retinoic acid inducible gene I receptor (RIG-I) agonist is exogenous.

As used herein, the terms “retinoic acid inducible gene I receptor (RIG-I) agonist” or “RIG-I agonist” refer to a substance that binds to RIG-I gene or protein (direct agonist), or a substance that binds to a gene or protein upstream of the RIG-I gene or protein (indirect agonist) that will lead to the activation of RIG-I and produce a biological response. Examples of RIG-I direct agonists include, but are not limited to, 3p-125b-ASO and immRNA. Examples of RIG-I indirect agonists include, but are not limited to, KRAS-G12D ASO. KRAS-G12D does not bind to RIG-I but can synergize with immRNA to promote RIG-I activation.

As used herein, the terms “immunomodulatory RNA” or “immRNA” refer to an RNA with a short hairpin RNA (shRNA) structure that binds to a target gene. For example, the immRNA binds to a gene of interest to activate retinoic acid inducible gene I receptor (RIG-I). The immRNA can comprise about 1-30 nucleotides, or about 7-11 nucleotides, about 10-14 nucleotides, about 13-17 nucleotides, about 16-20 nucleotides, about 19-23 nucleotides, about 22-26 nucleotides, about 25-30 nucleotides, or about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides.

As used herein, the terms “antisense oligonucleotide” or “ASO” refer to a single-stranded deoxyribonucleotide that is complementary to an RNA target, for example but not limited to, mRNA, microRNA, or non-long coding RNA. Antisense oligonucleotides silence (block) a target gene. For example, the antisense oligonucleotide binds to a gene of interest to silence one or more oncogene. In another example, a modified ASO can activate a gene of interest for example, the retinoic acid inducible gene I receptor (RIG-I). Exemplary modified ASOs can include, but are not limited to, an ASO with a 5′triphosphate cap that binds to miR-125b and/or RIG-I. The antisense oligonucleotide can comprise about 1-30 nucleotides, or about 1-5 nucleotides, about 4-8 nucleotides, about 7-11 nucleotides, about 10-14 nucleotides, about 13-17 nucleotides, about 16-20 nucleotides, about 19-23 nucleotides, about 22-26 nucleotides, about 25-30 nucleotides, or about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides.

As used herein, the term “siRNA” is also known in the art as “small interfering RNA”, and refers to a double-stranded RNA which is about 20 to 24 nucleotides in length. siRNA interferes with gene expression by binding to target mRNA via complementary base pairing and degrading the target mRNA.

As used herein, the term “antibody” refers to an immunoglobulin molecule capable of binding to a specific epitope on an antigen. Antibodies can be comprised of a polyclonal mixture, or may be monoclonal in nature. Further, antibodies can be entire immunoglobulins derived from natural sources, or from recombinant sources. The antibodies of the present disclosure may exist in a variety of forms, including for example as a whole antibody, or as an antibody fragment, or other immunologically active fragment thereof, such as complementarity determining regions. Antibodies may exist as an antibody fragment having functional antigen-binding domains, that is, heavy and light chain variable domains.

As used herein, the term “monoclonal antibody” refers to an antibody that recognises and binds to a single epitope.

As used herein, the term “single chain antibody” is used interchangeably with the terms “single chain Fv” and “scFv”, and refers to an antibody or immunoglobulin that comprises the variable regions from both the heavy and light chains which are connected by a peptide linker, and which lacks the constant regions.

As used herein, the term “nanobody” refers to an antibody that has the variable domain of the heavy chain only, and lacks the light chain. The antigen-binding capacity of nanobodies, however, remains similar to that of conventional antibodies and can bind to a target. Examples of nanobodies can be, but are not limited to, EGFR, HER1, HER2, HER3, human growth factor (HGF), CXCR4, PD-L1, CD49a, CD49b, CD49c, CD49d, CD49e, CD49f, ITGA7, ITGA8, ITGA9, ITGA10, ITGA11, CD11D, CD103, CD11a, CD11b, CD51, CD41, CD11c, CD29, CD18, CD61, CD104, ITGB5, ITGB6, ITGB, ITGB8, FOLR1, FOLR2 or FOLR3 binding nanobodies.

As used herein, the term “cancer marker”, is also known as a “tumour marker” in the art, and refers to any molecule which can be found in increased levels, compared to average levels, in a subject suffering from cancer. Cancer markers differ between cancers and are well-known by a person in the art, who will be aware of what molecules can be found in increased levels in a subject suffering from cancer. Examples of molecules can be, but is not limited to, Alpha fetoprotein (AFP), immunoglobulin, an epidermal growth factor receptor (EGFR), HER1, HER2, HER3, human growth factor (HGF), CXCR4, PD-L1, one or more integrin proteins (e.g. CD49a, CD49b, CD49c, CD49d, CD49e, CD49f, ITGA7, ITGA8, ITGA9, ITGA10, ITGA11, CD11D, CD103, CD11a, CD11b, CD51, CD41, CD11c, CD29, CD18, CD61, CD104, ITGB5, ITGB6, ITGB OR ITGB8), or folic acid receptors (e.g. FOLR1, FOLR2 or FOLR3). EGFR can be found in increased levels in cancers such as, but not limited to, breast cancer, lung cancer, pancreatic cancer, colorectal cancer, or prostate cancer. HER1/HER2/HER3 can be found in increased levels in cancers such as, but not limited to, breast cancer, bladder cancer, pancreatic cancer, ovarian cancer, or stomach cancer.

### DETAILED DESCRIPTION

In recent decades, there have been large strides in the development of immunotherapy beginning with the breakthrough approval of interferon-alpha2 anti-tumour cytokine by the U.S. Food and Drug Administration (FDA)

RIG-I is a cytosolic RNA sensor that recognizes RNA sequences with a 5′ triphosphate moiety and binds to short double-stranded RNAs with higher affinity. Although the role of RIG-I as a prognostic marker varies across cancer types, its activation consistently induces apoptotic tumour cell death and increases infiltration of immune cells into the tumour, enhancing their anticancer effects and reducing immunosuppressive activities.

Intratumoral delivery of RIG-I agonists induces apoptosis of pancreatic cancer cells in a type I IFN-dependent manner and enhances effective cross-presentation of tumour-associated antigens by dendritic cells to CD8+ T cells, therefore leading to tumour regression, for example, in mice with pancreatic cancer.

As RNA oligonucleotides including small interference RNAs (siRNAs) and antisense oligonucleotides are being developed to target disease genes, combining gene knockdown with RIG-I activation is one approach for anticancer therapy.

However, clinical application of RNA therapeutics continues to be restrained by current delivery of these molecules to target cells. One of the most widely used delivery vehicles of RIGI agonists is the polymer in vivo-jetPEI, where there have been contrasting reports on its safety. In vivo-jetPEI tends to induce liver damage and is associated with poor survival rates of mice, an effect attributed to its toxicity. Thus, developing a delivery method is crucial for RNA therapy.

In view of the above problems, there is a need to provide a composition that can be used for delivering a retinoic acid inducible gene I receptor (RIG-I) agonist to cell. The effective delivery of the a retinoic acid inducible gene I receptor (RIG-I) agonist will allow treatment of a disease.

Accordingly, the inventors of the present disclosure have found a composition comprising a vesicle for delivery of a retinoic acid inducible gene I receptor (RIG-I) agonist to a cell.

The retinoic acid inducible gene I receptor (RIG-I) agonist as used herein can be an immunomodulatory RNA (immRNA), an antisense oligonucleotide (ASO), a small interfering RNA (siRNA), or any combinations thereof.

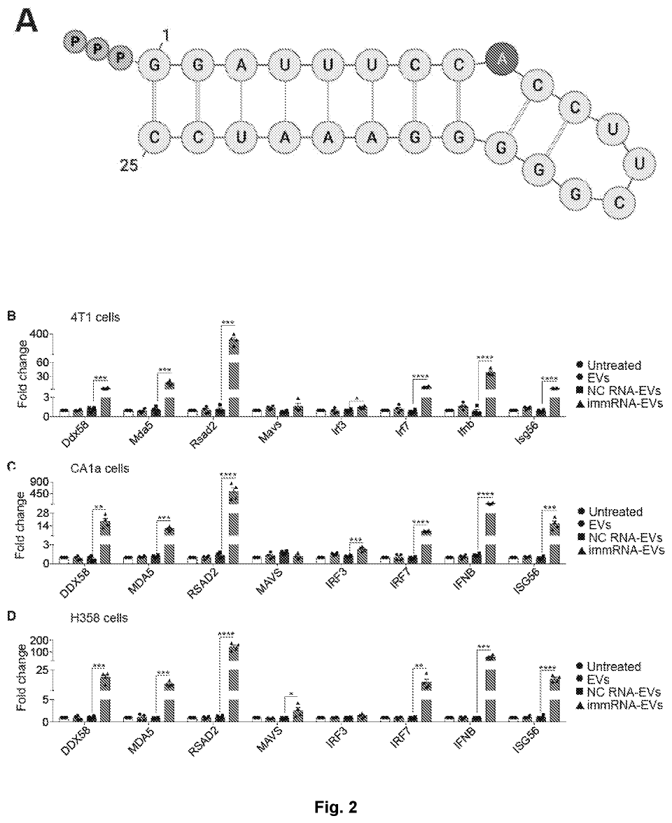

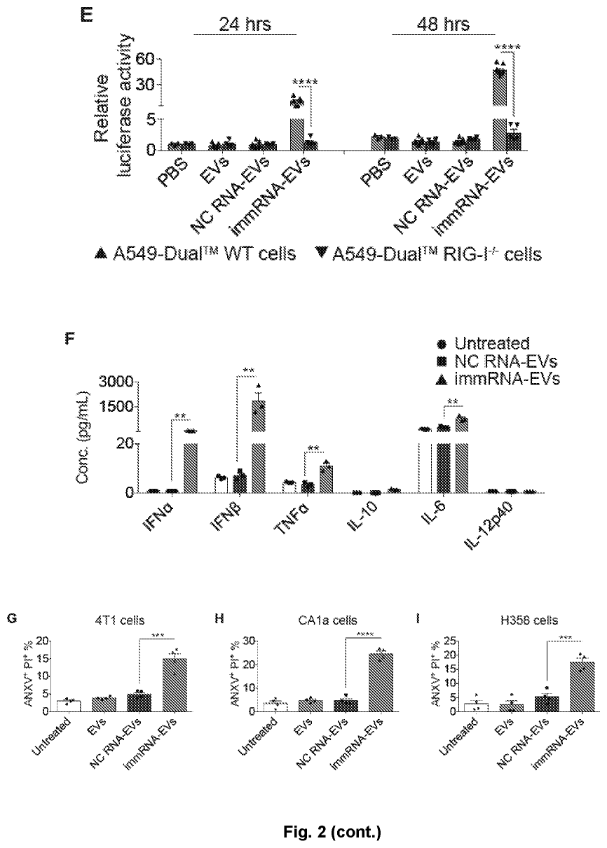

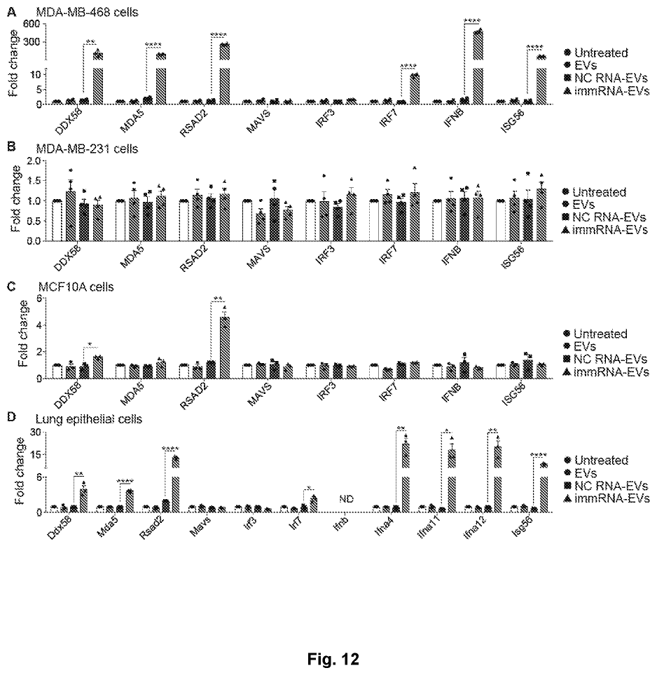

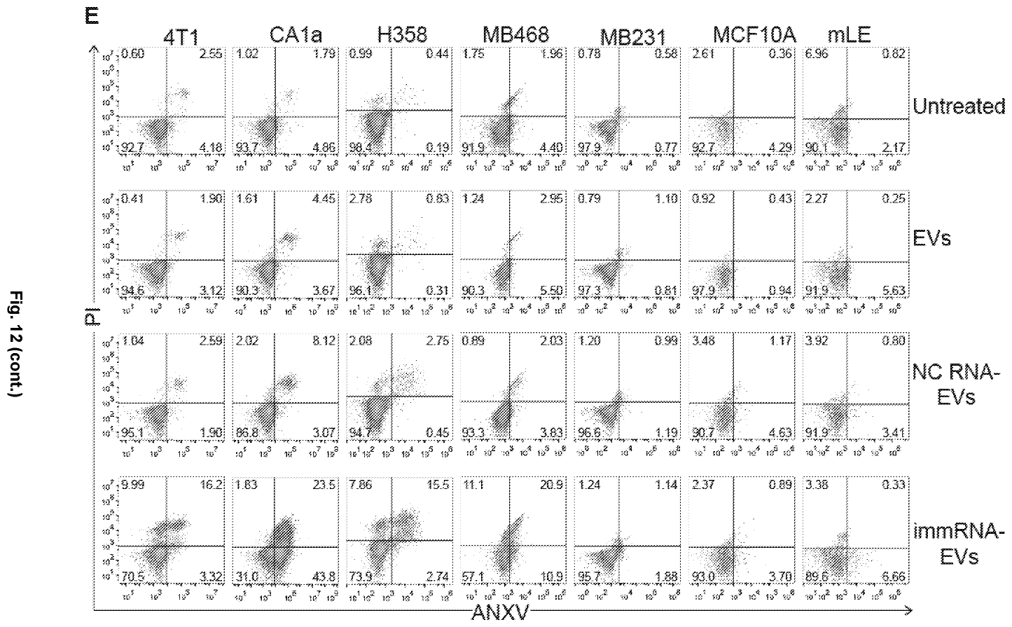

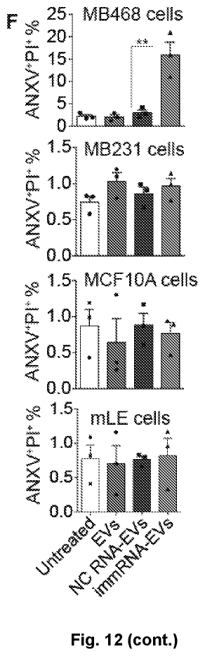

Immunomodulatory RNA (immRNA) can be delivered to cancer cells to trigger immunogenic cell death of cancer cells by activation of the RIG-I pathway (FIGS. 2 and 12). Thus, in one example, the retinoic acid inducible gene I receptor (RIG-I) agonist can be an immunomodulatory RNA (immRNA). In another example, the immunomodulatory RNA (immRNA) comprises the sequence 5′-GGAUUUCCACCUUCGGGGGAAAUCC-3′ (SEQ ID NO: 1).

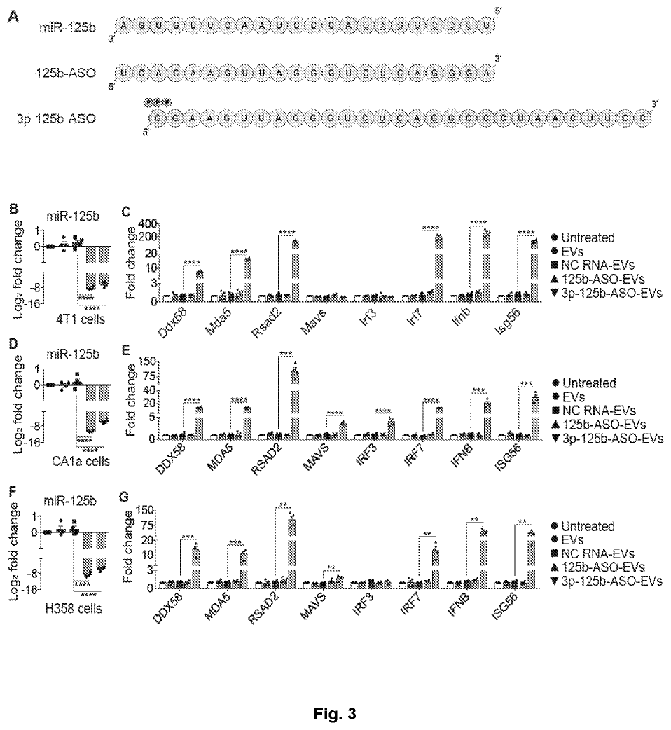

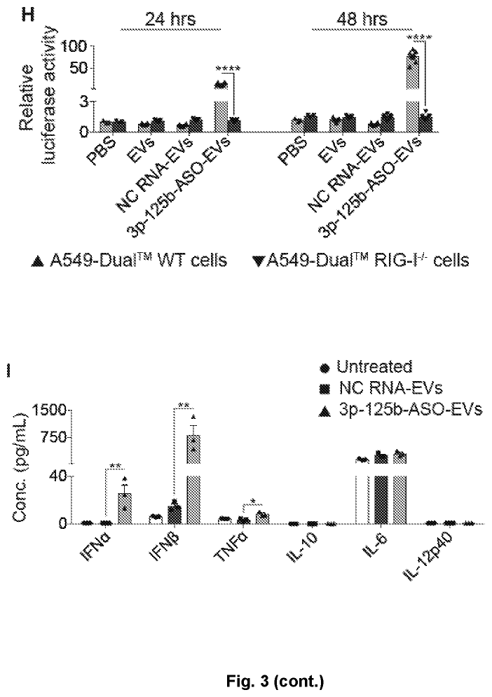

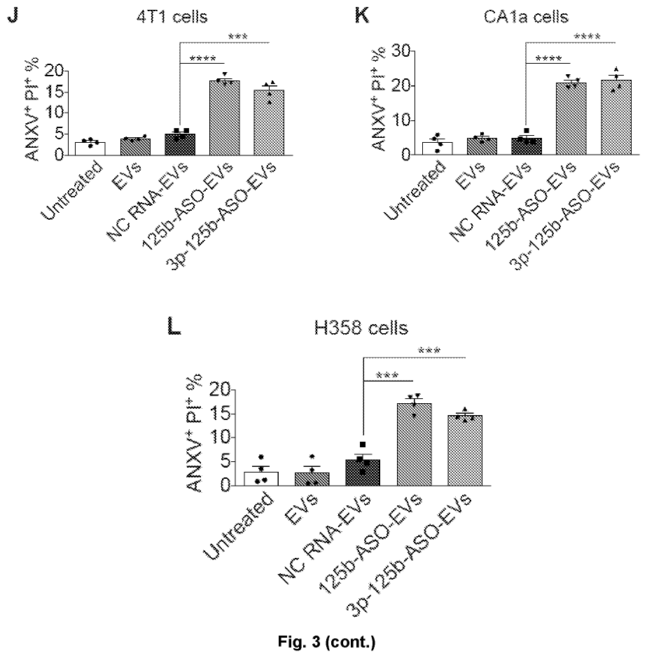

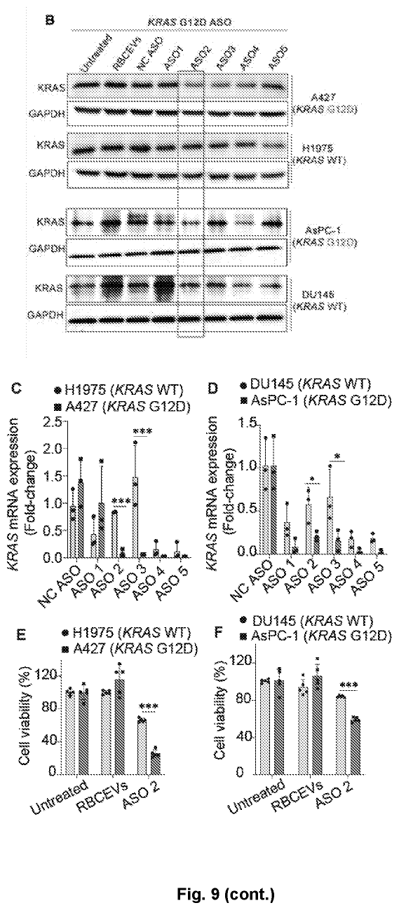

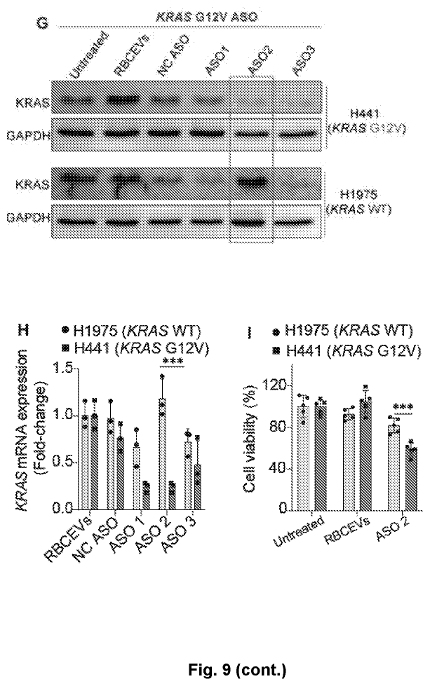

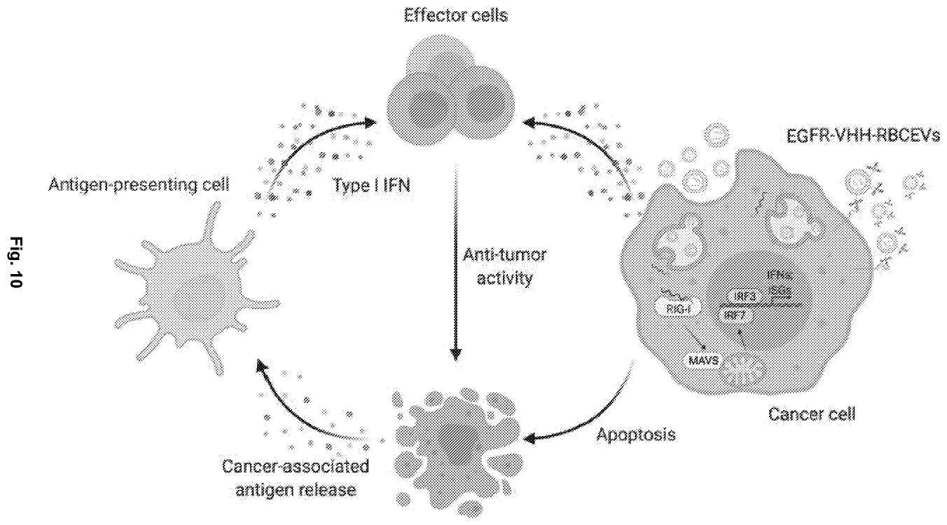

Antisense oligonucleotides (ASO) can also be delivered to cancer cells to trigger cell death in cancer cells (FIGS. 3 and 9). In addition, an ASO targeting KRAS-G12D and/or KRAS-G12V can synergise with RIG-I agonist to trigger cancer cell death (FIG. 9). Accordingly, in one example, the retinoic acid inducible gene I receptor (RIG-I) agonist can be an antisense oligonucleotide (ASO). In another example, the antisense oligonucleotide (ASO) can be a miRNA-125b-ASO, a KRAS-ASO, an EGFR targeting ASO, or any combinations thereof. In one example, the antisense oligonucleotide (ASO) is a miRNA-125b-ASO. In a further example, the antisense oligonucleotide (ASO) is a miRNA-125b-ASO comprising the sequence 5′-GGAAGUUAGGGUCUCAGGCCCUAACUUCC-3 (SEQ ID NO: 2). In another example, the antisense oligonucleotide (ASO) is a KRAS-ASO. In a further example, the retinoic KRAS-ASO can be a KRAS-G12D ASO, a KRAS-G12V ASO, a KRAS-G12C ASO, or a KRAS-G12S ASO. In yet another example, the KRAS-ASO is a KRAS-G12D ASO which can comprise or consist of any one of the sequences in the table below:

| SEQ | | | SEQ | |
|---|---|---|---|---|
| ID | | ASO sequence | ID | Target sequence |
| No. | Name | (5′ → 3′) | No. | (5′ → 3′) |
| 3 | KRAS G12D ASO 1 | CCATCAGCTCCAACTACCAC | 21 | GTGGTAGTTGGAGCTGATGG |
| 4 | KRAS G12D ASO 2 | GCCATCAGCTCCAACTACCA | 22 | TGGTAGTTGGAGCTGATGGC |
| 5 | KRAS G12D ASO 3 | CTTGCCTACGCCATCAGCTC | 23 | GAGCTGATGGCGTAGGCAAG |
| 6 | KRAS G12D ASO 4 | TCTTGCCTACGCCATCAGCT | 24 | AGCTGATGGCGTAGGCAAGA |
| 7 | KRAS G12D ASO 5 | CTCTTGCCTACGCCATCAGC | 25 | GCTGATGGCGTAGGCAAGAG |

In yet another example, the KRAS-ASO is a KRAS-G12V ASO which can comprise or consist of any one of the sequences in the table below:

| SEQ | | | SEQ | |
|---|---|---|---|---|
| ID | | ASO sequence | ID | Target sequence |
| No. | Name | (5 ′ → 3′) | No. | (5′ → 3′) |
| 8 | KRAS G12V ASO 1 | CAACAGCTCCAACTACCACA | 26 | TGTGGTAGTTGGAGCTGTTG |
| 9 | KRAS G12V ASO 2 | CCAACAGCTCCAACTACCAC | 27 | GTGGTAGTTGGAGCTGTTGG |
| 10 | KRAS G12V ASO 3 | ACTCTTGCCTACGCCAACAG | 28 | CTGTTGGCGTAGGCAAGAGT |

In another example, the antisense oligonucleotide (ASO) is an EGFR targeting ASO. In another example, the antisense oligonucleotide (ASO) is an EGFR L858R targeting ASO. In yet another example, the antisense oligonucleotide (ASO) is an EGFR L858R targeting ASO which can comprise or consist of any one of the sequences in the table below:

| SEQ | | | SEQ | |
|---|---|---|---|---|
| ID | | ASO sequence | ID | Target sequence |
| No. | Name | (5′ → 3′) | No. | (5′ → 3′) |
| 11 | EGFR L858R ASO 1 | GCCGCCCAAAATCTGTGATC | 29 | GATCACAGATTTTGGGCGGC |
| 13 | EGFR L858R ASO 2 | GGCCGCCCAAAATCTGTGAT | 30 | ATCACAGATTTTGGGCGGCC |
| 13 | EGFR L858R ASO 3 | TGGCCGCCCAAAATCTGTGA | 31 | TCACAGATTTTGGGCGGCCA |
| 14 | EGFR L858R ASO 4 | TTGGCCGCCCAAAATCTGTG | 32 | CACAGATTTTGGGCGGCCAA |

In another example, the antisense oligonucleotide (ASO) is an EGFR T790M targeting ASO. In yet another example, the antisense oligonucleotide (ASO) is an EGFR T790M targeting ASO which can comprise or consist of any one of the sequences in the table below:

| SEQ | | | SEQ | |
|---|---|---|---|---|
| ID | | ASO sequence | ID | Target sequence |
| No. | Name | (5′ → 3′) | No. | (5′ → 3′) |
| 15 | EGFR T790M | GGGCATGAGCTGCATGATGA | 33 | TCATCATGCAGCTCATGCCC |
| | ASO 1 | | | |
| 16 | EGFR T790M | AGGGCATGAGCTGCATGATG | 34 | CATCATGCAGCTCATGCCCT |
| | ASO 2 | | | |
| 17 | EGFR T790M | AAGGGCATGAGCTGCATGAT | 35 | ATCATGCAGCTCATGCCCTT |
| | ASO 3 | | | |
| 18 | EGFR T790M | GAAGGGCATGAGCTGCATGA | 36 | TCATGCAGCTCATGCCCTTC |
| | ASO 4 | | | |

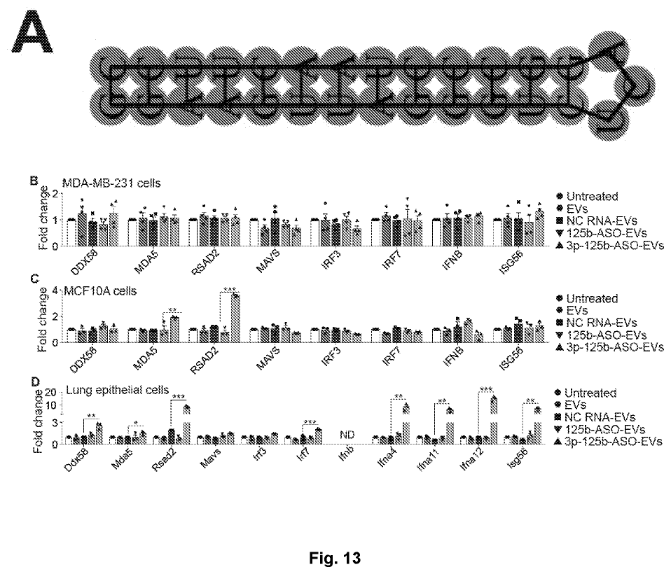

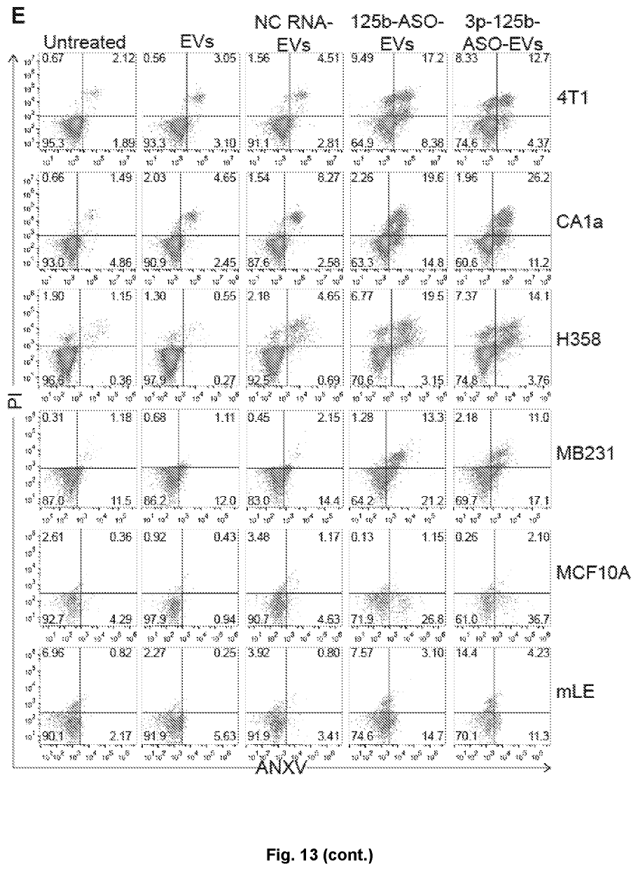

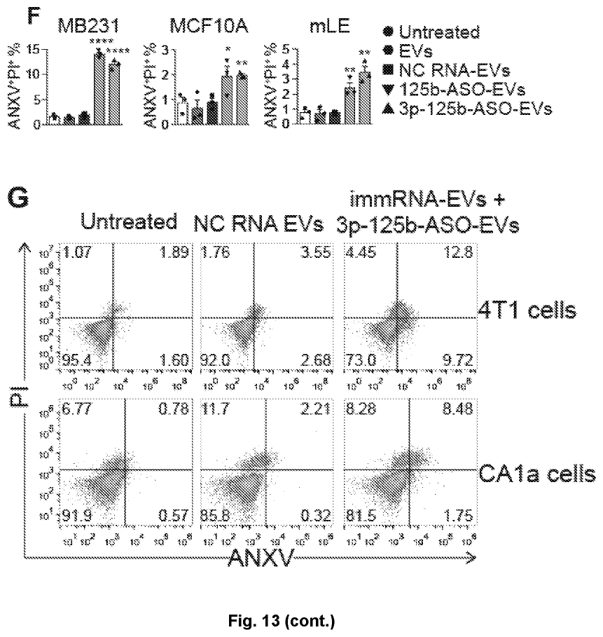

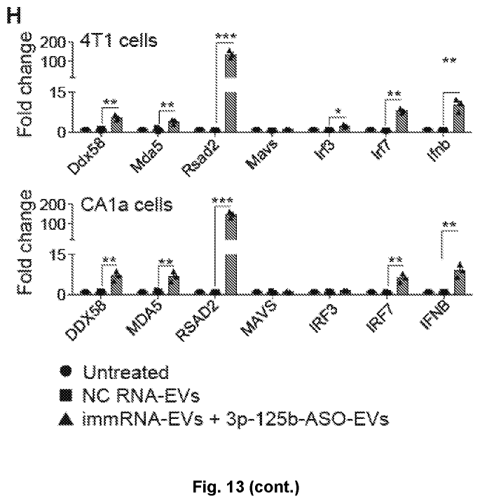

Modifications to the retinoic acid inducible gene I receptor (RIG-I) agonist can allow for multiple targets to be activated (FIGS. 3 and 13). Thus, in one example, the immunomodulatory RNA (immRNA) comprises a modification in the 5′ and/or 3′ end. In another example, the modification can be, but is not limited to, a 5′ triphosphate cap, phosphorylation, methylation, adenylation, or any combinations thereof. In a specific example, the modification is a 5′ triphosphate cap. Thus, in one example, the modified immunomodulatory RNA (immRNA) comprises the sequence 5′-pppGGAUUUCCACCUUCGGGGGAAAUCC-3′ (SEQ ID NO: 19).

In another example, the antisense oligonucleotide (ASO) comprises a modification in the 5′ and/or 3′ end. Suitable modifications can be, but are not limited to, a 5′ triphosphate cap, phosphorylation, methylation, adenylation, locked nucleic acid, phosphorothioate, 2-methoxyethyl, and combinations thereof. In a specific example, the modification is a 5′ triphosphate cap. Thus, in one example, the modified antisense oligonucleotide (ASO) can be a modified miRNA-125b-ASO, KRAS-ASO, EGFR targeting ASO, or any combinations thereof. In one example, the modified antisense oligonucleotide (ASO) is a modified miRNA-125b-ASO comprising the sequence 5′-pppGGAAGUUAGGGUCUCAGGCCCUAACUUCC-3′ (SEQ ID NO: 20). In yet another example, the modified antisense oligonucleotide (ASO) is a modified KRAS-ASO. In a further example, the modified KRAS-ASO can be a modified KRAS-G12D ASO, modified KRAS-G12V ASO, modified KRAS-G12C ASO, or modified KRAS-G12S ASO. In one example, the modified KRAS-ASO is a modified KRAS-G12D ASO which can comprise or consist of any one of the sequences in the table below:

| SEQ | | | SEQ | ASO sequence with |
|---|---|---|---|---|
| ID | Name | ASO sequence | ID | chemical modification |
| No. | | (5′ → 3′) | No. | (5′ → 3′) |
| 3 | KRAS G12D | CCATCAGCTCCAACTACCAC | 129 | +C*mC*mA*+T*mC*A*G*C*T*C |
| | ASO 1 | | | *C*A*A*C*T*mA*mC*mC*mA*+ |
| | | | | C |
| 4 | KRAS G12D | GCCATCAGCTCCAACTACCA | 130 | +G*mC*mC*mA*+T*C*A*G*C*T |
| | ASO 2 | | | *C*C*A*A*C*mT*mA*mC*mC*+ |
| | | | | A |
| 5 | KRAS G12D | CTTGCCTACGCCATCAGCTC | 131 | +C*mT*mT*mG*mC*C*T*A*C*G |
| | ASO 3 | | | *C*C*A*+T*C*mA*mG*mC*mT* |
| | | | | +C |
| 6 | KRAS G12D | TCTTGCCTACGCCATCAGCT | 132 | +T*mC*mT*mT*mG*C*C*T*A*C |
| | ASO 4 | | | *G*C*C*A*+T*mC*mA*mG*mC* |
| | | | | +T |
| 7 | KRAS G12D | CTCTTGCCTACGCCATCAGC | 133 | +C*mT*mC*mT*mT*G*C*C*T*A |
| | ASO 5 | | | *C*G*C*C*A*+T*mC*mA*mG*+ |
| | | | | C |

In another example, the modified KRAS-ASO is a modified KRAS-G12V ASO which can comprise or consist of any one of the sequences in the table below:

| SEQ | | | SEQ | ASO sequence with |
|---|---|---|---|---|
| ID | | ASO sequence | ID | chemical modification |
| No. | Name | (5′ → 3′) | No. | (5′ → 3′) |
| 8 | KRAS G12V | CAACAGCTCCAACTACCACA | 134 | +C*mA*+A*mC*mA*G*C*T*C*C |
| | ASO 1 | | | *A*A*C*T*A*mC*mC*mA*mC*+ |
| | | | | A |
| 9 | KRAS G12V | CCAACAGCTCCAACTACCAC | 135 | +C*mC*mA*+A*mC*A*G*C*T*C |
| | ASO 2 | | | *C*A*A*C*T*mA*mC*mC*mA*+ |
| | | | | C |
| 10 | KRAS G12V | ACTCTTGCCTACGCCAACAG | 136 | +A*mC*mT*mC*mT*T*G*C*C*T |
| | ASO 3 | | | *A*C*G*C*C*mA*+A*mC*mA*+ |
| | | | | G |

In another example, the modified antisense oligonucleotide (ASO) is a modified EGFR targeting ASO. In another example, the modified antisense oligonucleotide (ASO) is a modified EGFR L858R targeting ASO. In yet another example, the modified antisense oligonucleotide (ASO) is a modified EGFR L858R targeting ASO which can comprise or consist of any one of the sequences in the table below:

| SEQ | | | SEQ | ASO sequence with |
|---|---|---|---|---|
| ID | | ASO sequence | ID | chemical modification |
| No. | Name | (5′ → 3′) | No. | (5′ → 3′) |
| 11 | EGFR L858R | GCCGCCCAAAATCT | 137 | +G*mC*+C*mG*C*C*C*A*A*A*A |
| | ASO 1 | GTGATC | | *T*C*T*G*mT*mG*mA*mT*+C |
| 12 | EGFR L858R | GGCCGCCCAAAATC | 138 | +G*mG*mC*+C*mG*C*C*C*A*A* |
| | ASO 2 | TGTGAT | | A*A*T*C*T*mG*mT*mG*mA*+T |
| 13 | EGFR L858R | TGGCCGCCCAAAAT | 139 | +T*mG*mG*mC*+C*G*C*C*C*A* |
| | ASO 3 | CTGTGA | | A*A*A*T*C*mT*mG*mT*mG*+A |
| 14 | EGFR L858R | TTGGCCGCCCAAAA | 140 | +T*mT*mG*mG*mC*+C*G*C*C*C |
| | ASO 4 | TCTGTG | | *A*A*A*A*T*mC*mT*mG*mT*+G |

In another example, the modified antisense oligonucleotide (ASO) is a modified EGFR targeting ASO. In another example, the modified antisense oligonucleotide (ASO) is a modified EGFR T790M targeting ASO. In yet another example, the modified antisense oligonucleotide (ASO) is a modified EGFR T790M targeting ASO which can comprise or consist of any one of the sequences in the table below:

| SEQ | | | SEQ | ASO sequence with |
|---|---|---|---|---|
| ID | | ASO sequence | ID | chemical modification |
| No. | Name | (5′ → 3′) | No. | (5′ → 3′) |
| 15 | EGFR T790M | GGGCATGAGCTG | 141 | +G*mG*mG*mC*mA*T*G*A*G*C* |
| | ASO 1 | CATGATGA | | T*G*C*+A*T*mG*mA*mT*mG*+A |
| 16 | EGFR T790M | AGGGCATGAGCT | 142 | +A*mG*mG*mG*mC*A*T*G*A*G* |
| | ASO 2 | GCATGATG | | C*T*G*C*+A*mT*mG*mA*mT*+G |
| 17 | EGFR T790M | AAGGGCATGAGC | 143 | +A*mA*mG*mG*mG*C*A*T*G*A* |
| | ASO 3 | TGCATGAT | | G*C*T*G*C*+A*mT*mG*mA*+T |
| 18 | EGFR T790M | GAAGGGCATGAG | 144 | +G*mA*mA*mG*mG*G*C*A*T*G* |
| | ASO 4 | CTGCATGA | | A*G*C*T*G*mC*+A*mT*mG*+A |

Where:
+Locked Nucleic Acid (LNA)
*Phosphorothioated (PS)
m2′-O-methoxyethyl (2′-MOE)

Extracellular vesicles (EVs) derived from red blood cells (RBCs) are non-immunogenic and non-oncogenic. The absence of DNA in RBCs eliminates the risk of horizontal gene transfer with RBCEVs, a known risk of using EVs from nucleated cells for nucleic acid delivery; and immunogenicity can be prevented with blood type matching Furthermore, there is no observable toxicity associated with RBCEV treatmentTherefore, extracellular vesicles (EVs) for delivery of RIG-I's provide multiple advantages over existing RIG-I agonist delivery systems, due to the limited immunogenicity.

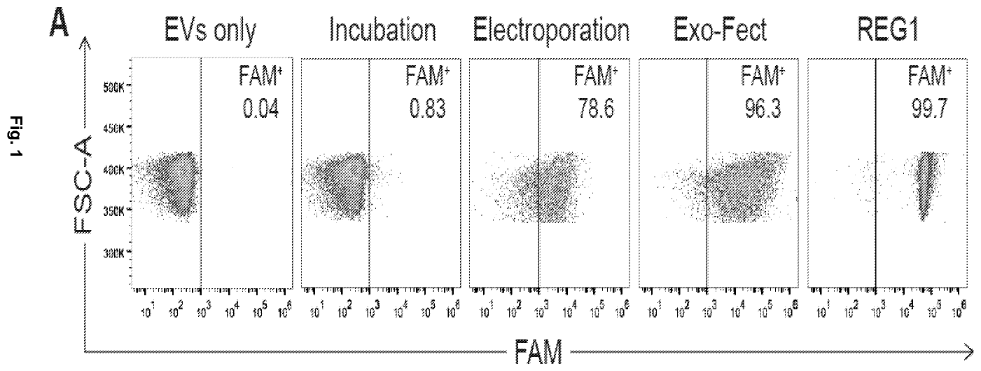

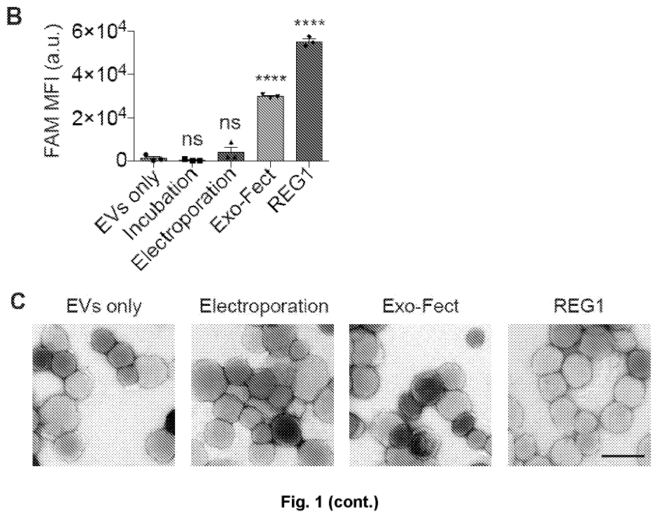

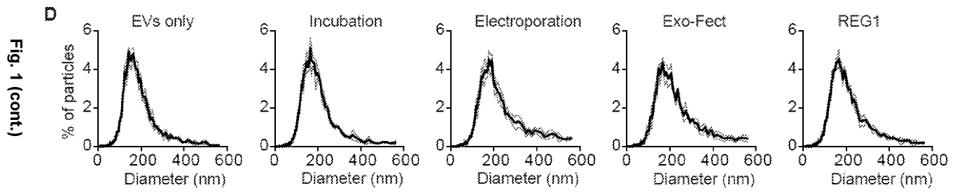

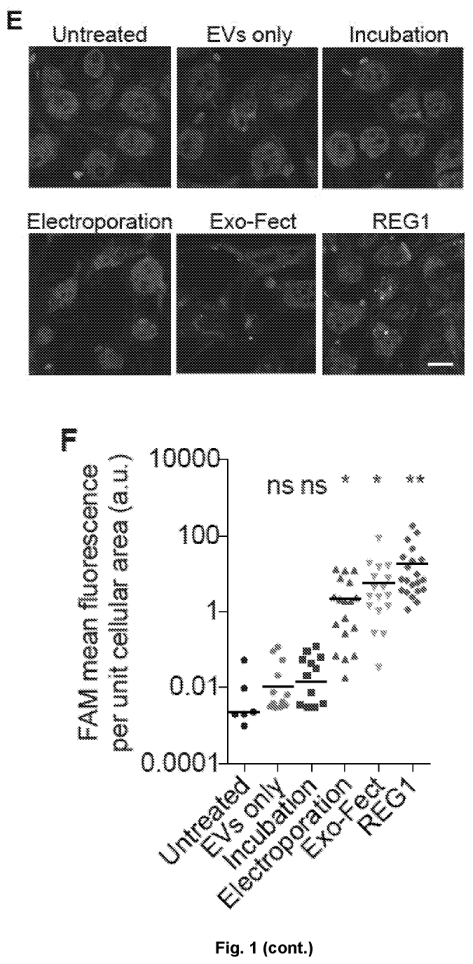

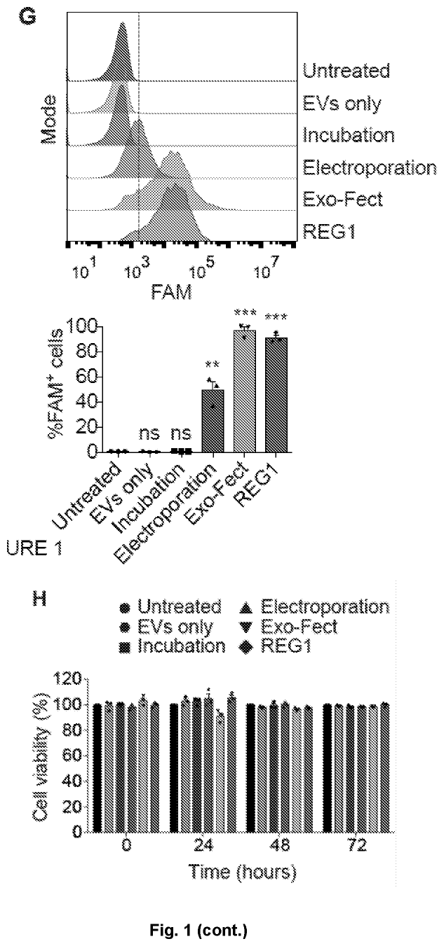

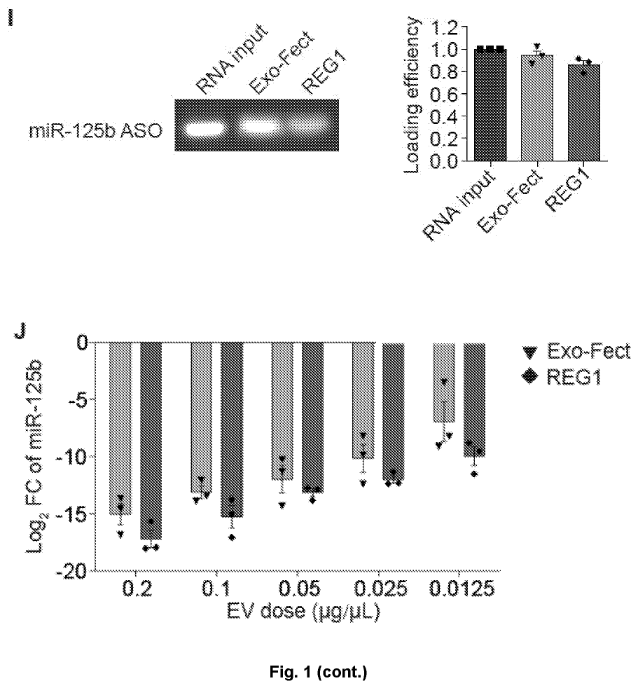

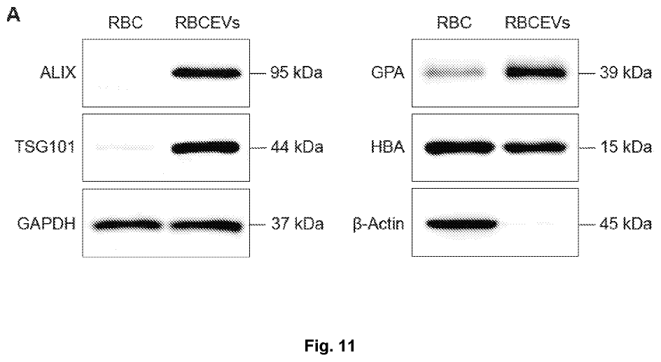

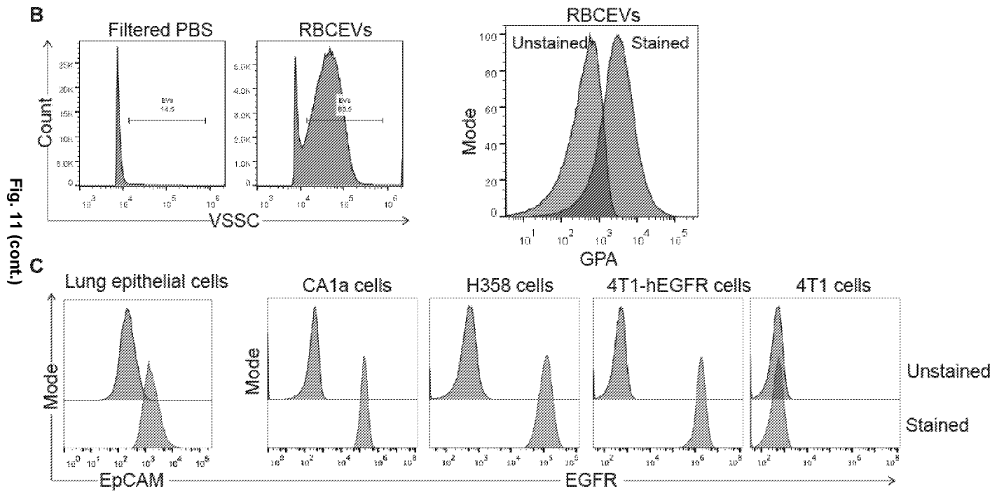

Accordingly, the composition as described herein can comprise an extracellular vesicle or a lipid nanoparticle for delivery of a retinoic acid inducible gene I receptor (RIG-I) agonist (FIGS. 1 and 11). Thus, in one example, the extracellular vesicle can be, but is not limited to, a red blood cell-derived extracellular vesicle (RBCEV), a milk derived EV, a plasmid derived EV, and a cancer cell derived EV. In a further example, the extracellular vesicle is a red blood cell-derived extracellular vesicle (RBCEV). In another example, the lipid nanoparticle comprises lipids which can be, but is not limited to, cationic lipids, ionizable lipids, PEG-lipids, phospholipids, phosphatidylcholine, phosphatidylethanolamine, or cholesterol. In one example, the lipid nanoparticle comprises PEG2000-modified lipid.

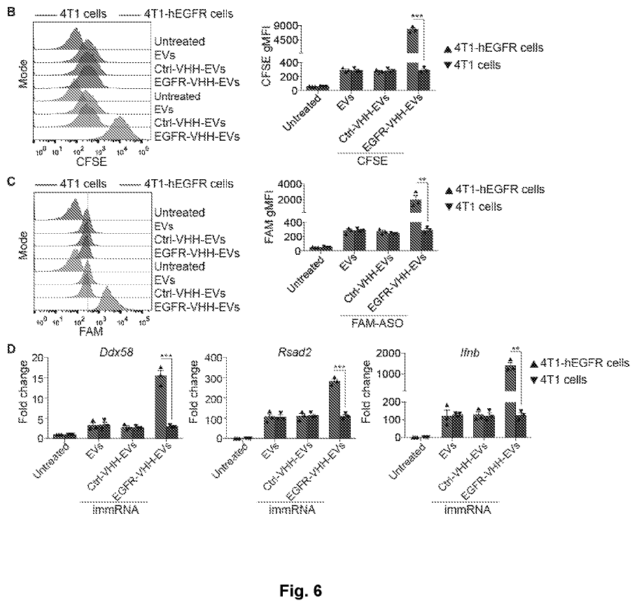

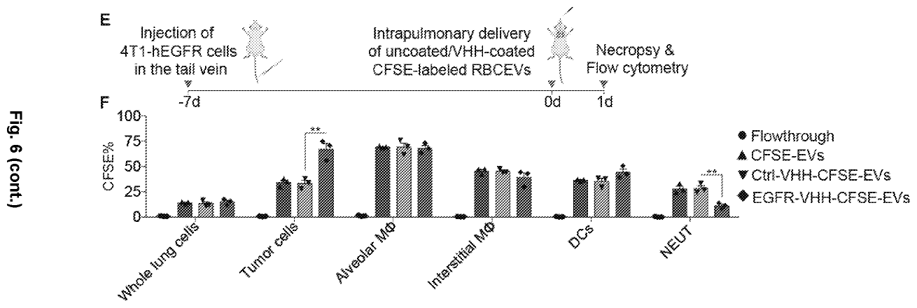

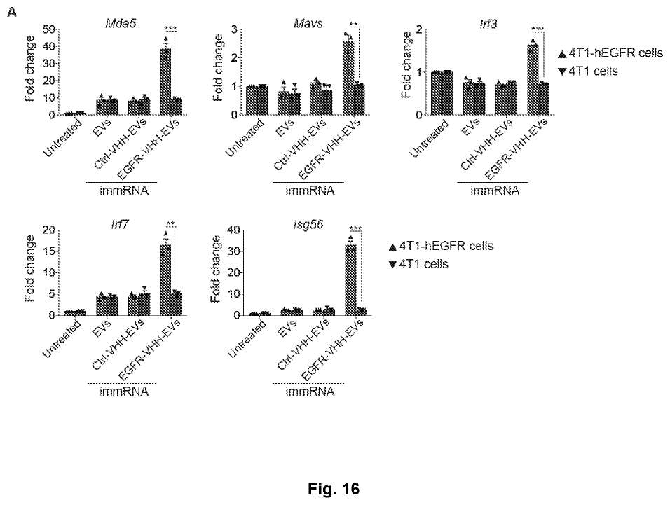

Targeting cancer cells specifically is an important characteristic of EV-based drug delivery to increase therapeutic efficacy and decrease toxicity. In the present invention, RBCEVs were conjugated to antibodies to facilitate uptake of extracellular vesicles by target cells (FIGS. 6 and 16). Thus, in one example, the antibody can be, but is not limited to, a nanobody, a monoclonal antibody, or a single-chain antibody. In another example, the antibody is a nanobody. In yet another example, the antibody is a nanobody that binds to a cancer marker. Accordingly, in one example, the cancer marker can be, but is not limited to, an epidermal growth factor receptor (EGFR), HER1, HER2, HER3, human growth factor (HGF), CXCR4, PD-L1, one or more integrin proteins (e.g. CD49a, CD49b, CD49c, CD49d, CD49e, CD49f, ITGA7, ITGA8, ITGA9, ITGA10, ITGA11, CD11D, CD103, CD11a, CD11b, CD51, CD41, CD11c, CD29, CD18, CD61, CD104, ITGB5, ITGB6, ITGB OR ITGB8), one or more folic acid receptors (e.g. FOLR1, FOLR2 or FOLR3), or combinations thereof.

In this disclosure, new compositions of EVs and RIG-I agonists are provided, which are capable of activating RIG-I, inducing anti-tumor immunity, in particular, type I interferon-mediated anti-tumor polarization, and targeting metastatic EGFR-positive cancer.

Thus, in one example, disclosed herein is a composition comprising:
a red blood cell-derived extracellular vesicle (RBCEV);
an immunomodulatory RNA (immRNA) comprising or consisting of 5′-GGAUUUCCACCUUCGGGGGAAAUCC-3′ (SEQ ID NO: 1), wherein the immunomodulatory RNA (immRNA) further comprises a 5′ triphosphate cap; and/or
an antisense oligonucleotide (ASO) comprising or consisting of 5′-GGAAGUUAGGGUCUCAGGCCCUAACUUCC-3′ (SEQ ID NO: 2), wherein the antisense oligonucleotide (ASO) further comprises a 5′ triphosphate cap (e.g. 3p-125b-ASO).

In another example, disclosed herein is a composition comprising:
a lipid nanoparticle;
an immunomodulatory RNA (immRNA) comprising or consisting of 5′-GGAUUUCCACCUUCGGGGGAAAUCC-3′, wherein the immunomodulatory RNA (immRNA) further comprises a 5′ triphosphate cap; and/or
a KRAS-ASO.

The compositions as disclosed herein can be formulated to be suitable for administration. Thus, in one example, disclosed herein is a pharmaceutical composition comprising the composition as disclosed herein and a a pharmaceutically acceptable excipient. Suitable excipients are well-known by a skilled person of the art and can be, but are not limited to, polymers, lactose, sucrose, or sodium starch glycolate.

The composition or pharmaceutical composition as disclosed herein can be packaged in a kit for a user's ease of use. Such kits can contain, the composition disclosed herein, or the pharmaceutical composition disclosed herein. Further ingredients of the kit may include other components which can be, but are not limited to, anti-cancer drugs, or buffers. Suitable anti-cancer drugs can be, but is not limited to, an anti-PD1 antibody, anti-oncogene ASOs, anti-cancer cytokines, and combinations thereof.

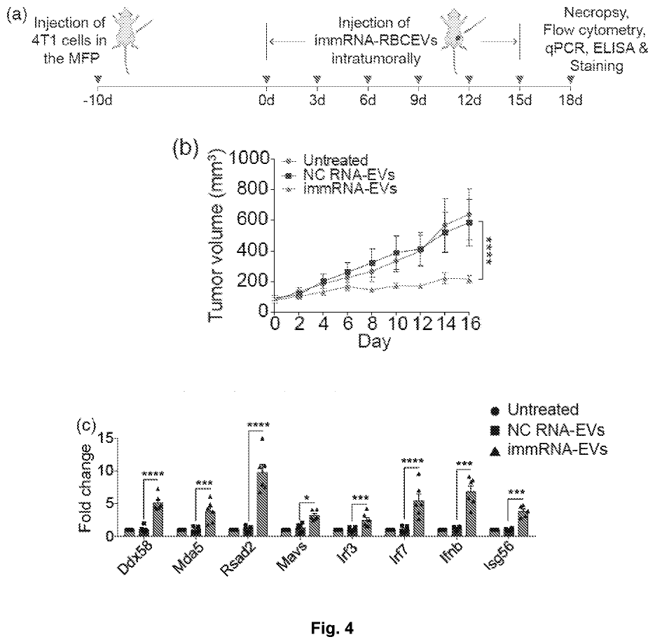

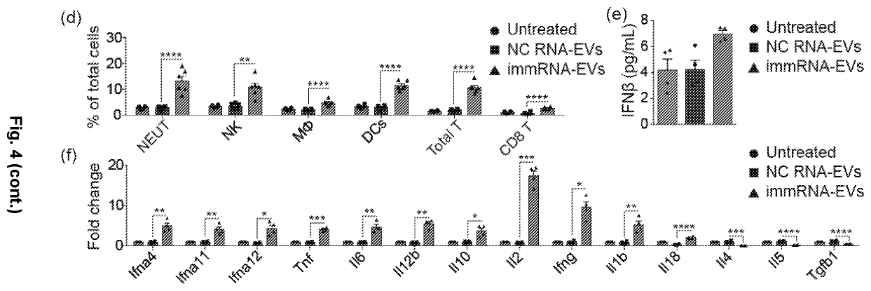

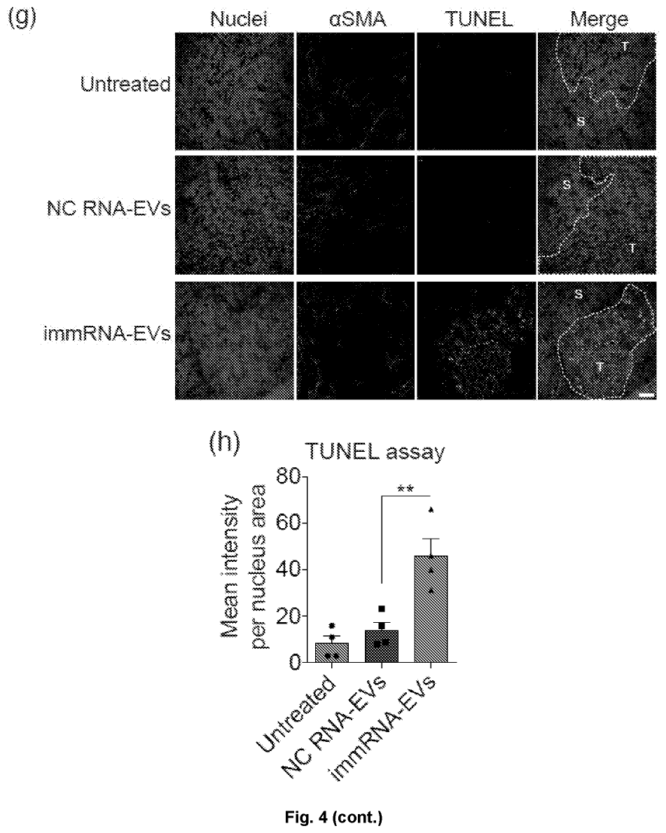

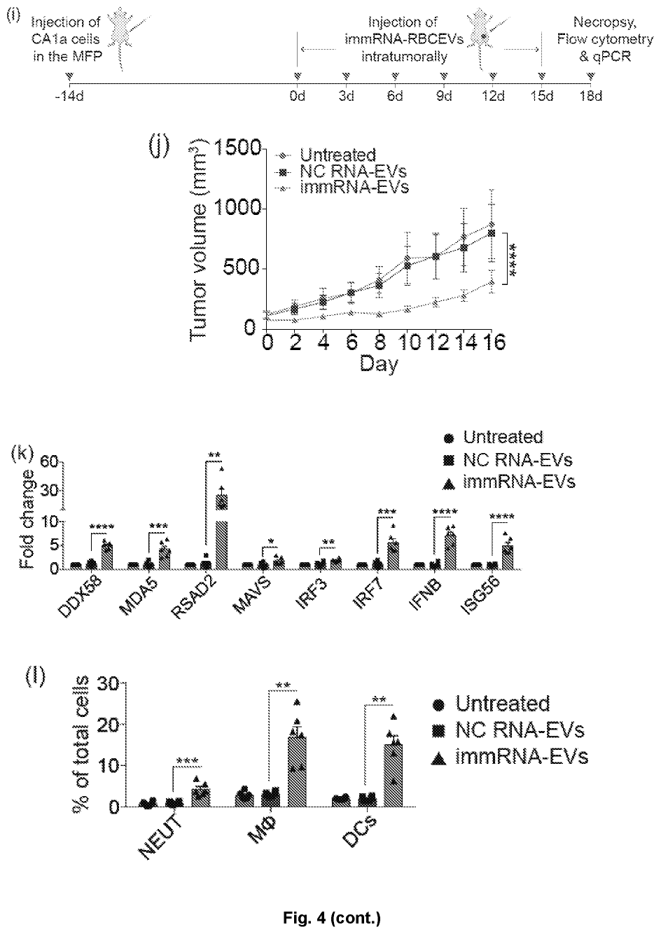

Effective immunomodulation of the tumor microenvironment and anti-tumor activity was achieved using composition described herein without any observable adverse effects, suggesting the advantages of the composition described herein for clinical application. For example, the treatment with RIG-I-agonist loaded EVs was validated in breast cancer and lung metastatic models (FIGS. 4, 5, 7, and 17).

In another example, delivery of a RIG-I agonist and an antisense oligonucleotide (ASO) was demonstrated to induce RIG-I pathway activation, production of type I-IFN, and apoptosis in cancer cells. In addition, immRNA also synergizes with KRAS-G12D ASO and KRAS-G12V ASO in suppressing the viability of cancer cells.

Accordingly, in one example, disclosed herein is a method of delivering RIG-I agonists using an extracellular vesicle. In another example, disclosed herein is a method of providing/delivering an anti-cancer therapy or an anti-cancer immunotherapy to a subject in need thereof.

Methods of delivery can be carried out by administering the composition, the pharmaceutical composition, or components of the kit of the disclosure. In one example, the composition as disclosed herein, or the pharmaceutical composition as disclosed herein, can be administered with an anti-cancer drug. The anti-cancer drug can be, but is not limited to, an anti-PD1 antibody, one or more anti-oncogene ASOs, and one or more anti-cancer cytokines. In another example, the anti-cancer drug can be administered simultaneously or subsequently with the composition as described herein. Suitable modes of administration are well-known by a person skilled in the art. Thus, in one example, methods of administration can be, but is not limited to, intrapulmonary, intranasal, intratracheal, subcutaneous, intravenous, intramuscular, intra-articular, intra-synovial, intrasternal, intrathecal, intrahepatic, intracranial injection, infusion techniques, topically, orally, rectally, nasally, buccally, and vaginally. In one example, the method of administration is intrapulmonary. In another example, the method of administration is intratumoral.

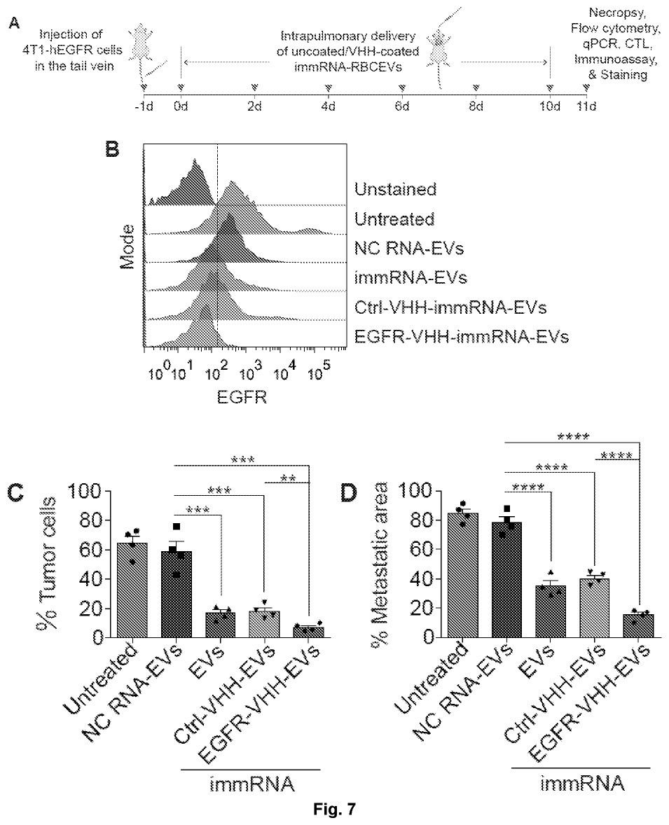

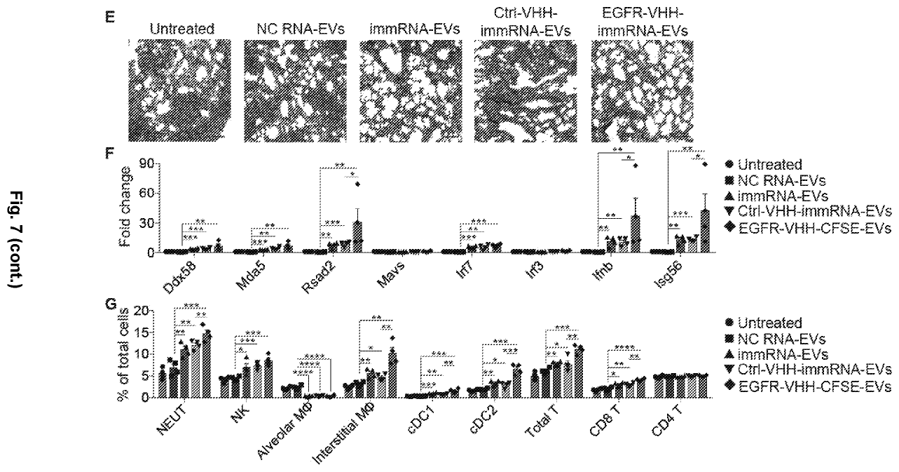

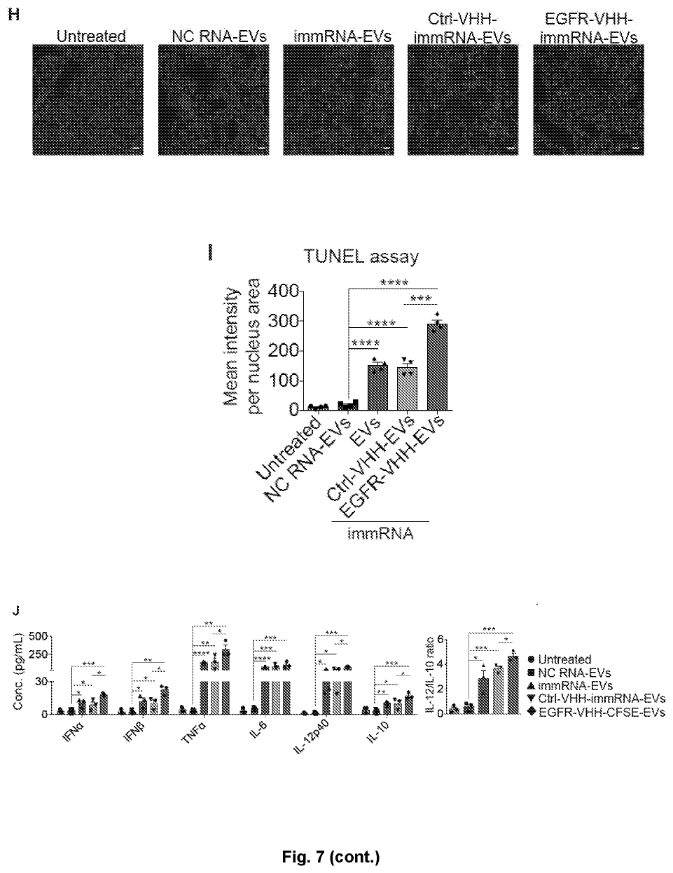

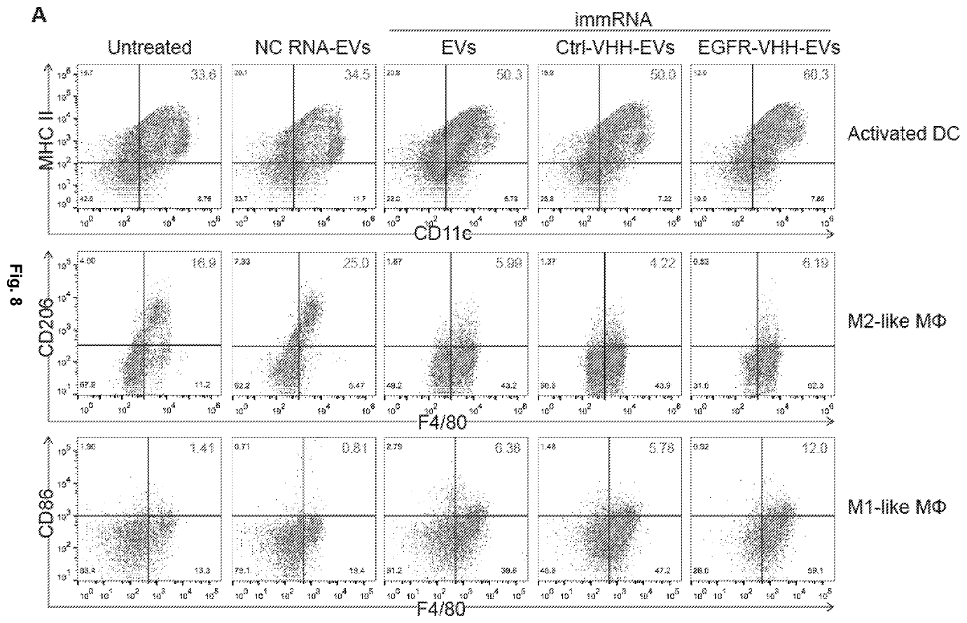

Intratumoral administration of RBCEVs comprising or encapsulating an RIG-I agonist and an antisense oligonucleotide (ASO) suppressed tumour growth and triggered high levels of type I IFN in the tumour microenvironment, immune cell activation and profound tumour cell apoptosis, and targeted delivery conferred potent anti-tumour efficacy (FIGS. 7, 8, and 17).

Accordingly, disclosed herein is a method of treating a disease with the composition, the pharmaceutical composition, or the kit of the disclosure to a subject in need thereof.

In one example, the disease is cancer. In another example, the cancer can be, but is not limited to, breast cancer, lung cancer, pancreatic cancer, brain cancer, head and neck cancer, liver cancer, stomach cancer, colon cancer, prostate cancer, cervical cancer, and bone cancer. In another example, the cancer is EGFR positive breast cancer. In another example, the cancer is EGFR positive lung cancer. In another example, the cancer is lung metastatic cancer. In another example, the cancer is pancreatic cancer.

In yet another example, the disease is a pulmonary disease or a disorder. In another example, the pulmonary disease or disorder can be, but is not limited to, asthma or pneumonia.

Also disclosed herein is a composition, a pharmaceutical composition, or a kit for use in therapy, or for use in treating a disease. In one example, disclosed herein is a composition, a pharmaceutical composition, or a kit for use in an anti-cancer therapy or an anti-cancer immunotherapy.

In another example, disclosed herein is a use of the composition, the pharmaceutical composition, or the kit of the disclosure in the manufacture of a medicament for treating a disease.

As used in this application, the singular form “a,” “an,” and “the” include plural references unless the context clearly dictates otherwise. For example, the term “a genetic marker” includes a plurality of genetic markers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Material and Methods

### Cell Culture

Mouse breast cancer 4T1 cell line and human breast cancer MCF10CA1a (CA1a) cell line were purchased from Karmanos Cancer Institute (Wayne State University, USA). Human breast cancer MDA-MB-468 and MDA-MB-231 cell lines, human untransformed mammary gland epithelial cell line MCF10A, human lung cancer NCI-H358 (H358) cell line, H1975, H441, and A427 cell lines, human pancreatic cancer AsPC-1 cells and human prostate cancer DU145 cell line, human monocytic THP-1 cells, and murine colorectal carcinoma CT26 cell line were obtained from the American Type Culture Collection (ATCC, USA). Human lung epithelial carcinoma reporter cell lines A549-Dual™ and A549-Dual™ RIGI^{-/-} were purchased from InvivoGen, USA. All cell lines except MCF10A cells were cultured in Dulbecco's Modified Eagle Media—DMEM (Thermo Fisher Scientific, USA) supplemented with 10% fetal bovine serum (FBS) (Biosera, France), 1×penicillin-streptomycin (Thermo Fisher Scientific), and 5 μg/ml Plasmocin™ prophylactic (InvivoGen, USA) in a humidified incubator at 37° C. with 5% CO2. MCF10A cells were cultured in MEBM medium supplemented with the additives from the MEGM kit (Lonza, UK) and 100 ng/ml cholera toxin (Sigma, USA).

### Mouse Primary Lung Epithelial Cell Isolation

Mouse lung epithelial cells were isolated from female BALB/c mice as previously described (Kasinski & Slack, 2013). Briefly, 7-week-old mice were euthanized. Mouse lungs were excised and dissociated in RPMI media containing 10% FBS and 5 mg/ml collagenase IV (Thermo Fisher Scientific) using the GentleMACS dissociator (Miltenyi Biotec, Germany). Cells were filtered through a 70 μm strainer, washed and seeded in a 10-cm plate. Fibroblasts are more sensitive to trypsin and can therefore be removed while the epithelial cells adhere for a longer time. Over the course of epithelial cell selection, removing the first cells that begin to slough off the plate upon trypsinization helps increase the purity of epithelial cells. EpCAM expression of epithelial cells was assessed using flow cytometry after three rounds of selection.

### Purification of RBCEVs

Blood samples of healthy donors with informed consent were obtained from the Singapore Health Science Authority and Hong Kong Red Cross. RBCs were separated from whole blood and RBCEVs were purified from RBCs as described previously (Pham et al., 2021). Briefly, RBCs were separated from plasma using centrifugation at 1,000×g for 8 min at 4° C. and washed three times with PBS (1,000×g for 8 min at 4° C.). White blood cells were completely removed by leukodepletion filters (Nigale, China). Isolated RBCs were collected in Nigale buffer (0.2 g/L citric acid, 1.5 g/L sodium citrate, 7.93 g/L glucose, 0.94 g/L sodium dihydrogen phosphate, 0.14 g/L adenine, 4.97 g/L sodium chloride, 14.57 g/L mannitol), diluted in PBS containing 0.1 mg/mL calcium chloride, and incubated overnight with 10 μM calcium ionophore (Sigma, USA) at 37° C. with 5% CO2. RBCs and cell debris were pelleted and the supernatant was collected by centrifugation at increasing speed (600×g for 20 min, 1,600×g for 15 min, and 3,260×g for 15 min). The supernatant was filtered through a 0.45 μm membrane before ultracentrifugation at 50,000×g for 70 min at 4° C. using a SW32 rotor (Beckman Coulter, USA). The pellet was resuspended in 1 mL of PBS and subsequently loaded onto a 60% sucrose cushion and ultracentrifuged at 50,000×g for 16 hours at 4° C. RBCEVs were collected at the interface and washed with PBS at 50,000×g for 70 min at 4° C. Purified RBCEVs were stored in PBS containing 4% trehalose at −80° C.

### Characterization of RBCEVs

The size distribution and concentration of RBCEVs were quantified using a ZetaView® nanoparticle tracking analyzer (Particle Metrix, Germany). Because hemoglobin is the major constituent of RBCEVs, RBCEV quantity is indicated by hemoglobin quantity throughout this study. The hemoglobin contents of RBCEVs were quantified using a NanoDrop™ 2000 spectrophotometer (Thermo Fisher Scientific).

### Western Blot Analysis

RBCEVs were lysed with RIPA buffer (Thermo Fisher Scientific) supplemented with protease inhibitors (Biotool, USA) for 5 min on ice. Cells were lysed for 30 min on ice. Protein concentration was measured by a Pierce™ BCA assay (Life Technologies, USA) with BSA (New England Biolabs, UK) concentration as standards. A total of 50 μg protein lysates were loaded onto 10% polyacrylamide gels together with a Precision Plus Protein™ Kaleidoscope™ prestained protein standard (Bio-Rad, USA). The proteins were transferred to Immobilon-P polyvinylidene difluoride membranes (Merck Millipore, USA), which were blocked using 5% milk in Tris buffered saline containing 0.1% Tween-20 (TBST) for 1 hour followed by an incubation with primary antibody, anti-ALIX antibody (Santa Cruz, USA, dilution 1:1,000), anti-TSG101 antibody (Santa Cruz, dilution 1:1,000), anti-human GAPDH antibody (Santa Cruz, dilution 1:1,000), antihuman GPA antibody (Santa Cruz, dilution 1:500), anti-human HBA antibody (Santa Cruz, dilution 1:1,000), anti-human KRAS antibody (Invitrogen, USA, dilution 1:1,000), anti-EGFR antibody (Santa Cruz, USA, dilution 1:1,000) or antihuman β-actin antibody (CST, USA, dilution 1:1,000) overnight at 4° C. The membranes were washed three times with TBST then incubated with HRP-conjugated secondary antibody (Jackson ImmunoResearch, USA, dilution 1:10,000) for 1 hour at room temperature. The blots were imaged using a ChemiDoc™ gel documentation system (Bio-Rad).

### Transmission Electron Microscopy

RBCEVs were fixed with 2% paraformaldehyde for 10 min and loaded on a glow-discharged copper grid (200 mesh, coated with formvar carbon film). RBCEVs on the grid were incubated with 3% uranyl acetate for 5 min to perform negative staining of RBCEVs. This was followed by a quick wash with distilled water to remove excess stain. The grids were air dried for 10 min before being imaged using a Tecnai G2 transmission electron microscope (FEI) at 100 kV.

### Single-EV Flow Cytometry

Single-EV flow cytometry was carried out using a CytoFLEX LX flow cytometer (Beckman Coulter, USA). RBCEVs were stained with anti-human GPA-FITC antibody (BioLegend, USA) for 3 hours at 4° C., followed by two washing steps using sterile 0.2 μm-filtered PBS to remove unbound antibody. It was previously found that EV suspensions within the range of 3.9×10³ to 2.5×10⁵ EVs/μL allowed analysis of single EVs accurately (Pham et al., 2021). As such, the stained RBCEVs were diluted to a concentration of 3×10⁴ EVs/μL in 0.2 μm-filtered PBS. RBCEVs were gated out from background noise using the violet side scatter (VSSC) channel acquired via the 405 nm laser (FIG. 11B). Reference 100 nm beads were used to verify the gating of the EV population. Data was acquired using the following settings: FSC 138, SSC 180, FITC 3000, VSSC 800 with the threshold of the trigger signal (VSSC) set manually to 4000. The flow rate was maintained at 30 μL/min (achieving an event rate of ˜5000 events/second) and the abort rate kept below 5% for the duration of the experiment.

### Sequences and Modifications of RNA Oligonucleotides

immRNA (5′-pppGGAUUUCCACCUUCGGGGGAAAUCC-3′) (SEQ ID NO: 19) and 5 triphosphate 125b-ASO (3p-125b-ASO) (5′-pppGGAAGUUAGGGUCUCAGGCCCUAACUUCC-3′) (SEQ ID NO: 20) were synthesized by in vitro transcription, as described in the next section. Anti-miR-125b ASO (125b-ASO) (5′-UCACAAGUUAGGGUCUCAGGGA-3′) (SEQ ID NO: 53), negative control RNA (NC RNA) (5′-CAGUACUUUUGUGUAGUACAA-3′) (SEQ ID NO: 54) and FAM-labeled NC-ASO (5′-FAMCAGUACUUUUGUGUAGUACAA-3′) (SEQ ID NO: 55) were synthesized with 2′ O-methyl modification at every ribonucleotide by Shanghai GenePharma (Shanghai, China).

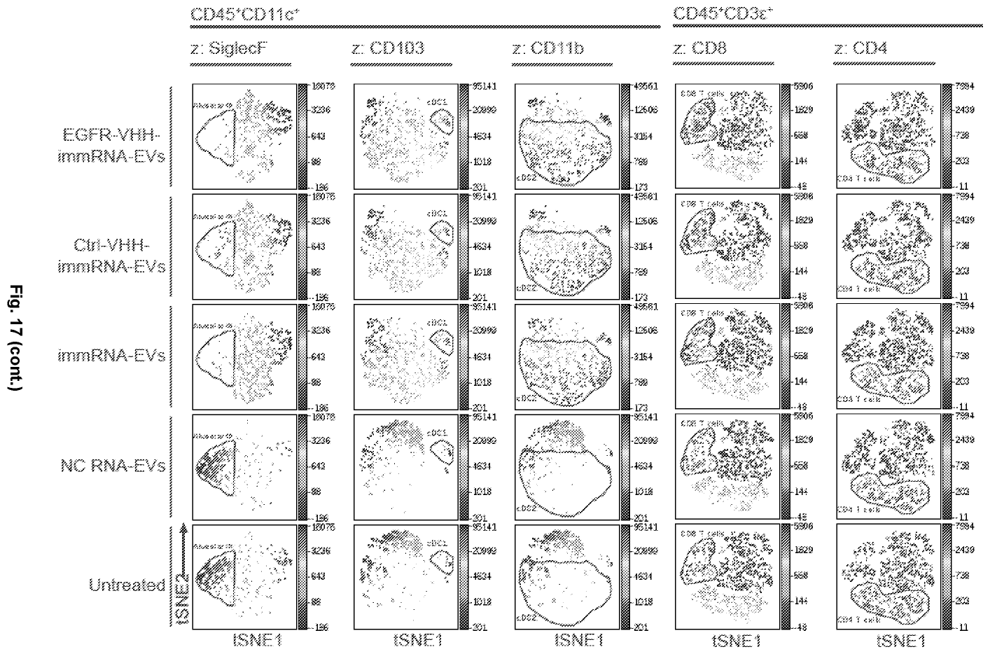

Oncogene-targeting ASOs with 2′-O-methoxyethyl (MOE), Phosphorothioate (PS), and Locked Nucleic Acid (LNA) modifications (FIGS. 9A and 18A) were synthesized by Integrated DNA Technologies (USA).

### In Vitro Transcription (IVT) of RNA

immRNAs were prepared following the protocol of Yong et al., 2019. Briefly, RNAs were transcribed in vitro using T7 RNA polymerase and a pair of primers (Forward: GGATTTCCCCCGAAGGTGGAAATCCTATAGTGAGTCGTATTAC (SEQ ID NO: 125); Reverse: GTAATACGACTCACTATAGGATTTCCACCTTCGGGGGAAATCC) (SEQ ID NO: 126). The reactions contain 40 mM Tris-HCl pH 8.0, 30 mM MgCl2, 2 mM spermidine, 10 mM DTT, 0.01% Triton-X100, 5 mM GTP, and 4 mM NTP (CTP, ATP and UTP), 1 μM annealed DNA template, 400-600 nM T7 RNA polymerase, and 0.2 U/mL thermostable inorganic pyrophosphatase, which react overnight at 37° C. The phenol:chloroform:isoamyl alcohol (25:24:1) was used to stop IVT reactions and extract RNAs. The RNAs were precipitated overnight at −80° C. with three volumes of 95% ethanol in presence of 0.1% (v/v) of sodium acetate. The target RNAs were further isolated by a Hi-TrapQ HP column and the expected bands of RNAs were excised from a 20% denaturing urea-PAGE. Similar to immRNAs, 3p-125b-ASOs were prepared as described above using a DNA template (Sense: 5′-GTAATACGACTCACTATAGGAAGTTAGGGTCTCAGGCT-3′ (SEQ ID NO: 56); Antisense: 3′-CATTATGCTGAGTGATATCCTTCAATCCCAGAGTCCGA-5′ (SEQ ID NO: 57)) and of (Forward: a pair primers AGCCTGAGACCCTAACTTCCTATAGTGAGTCGTATTAC (SEQ ID NO: 127); Reverse: GTAATACGACTCACTATAGGAAGTTAGGGTCTCAGGCT (SEQ ID NO: 128)). The quality of immRNAs and 3p-125b-ASOs were determined again on a 20% denaturing urea-PAGE and by IFNs activity assay prior to the subsequent experiments.

To confirm the sequence of 3p-125b-ASO, the ASO was incubated with RNA 5′ pyrophosphohydrolase (RppH) (New England Biolabs) for 1-2 hours at 37° C. The RNA was analysed using BioAnalyzer 2100 (Agilent, USA) and converted to cDNA using a NEBNext small RNA library prep kit (New England Biolabs) according to the manufacturer's instructions. The library was sequenced using Illumina HiSeq 2500 system. Sequencing reads were processed using Geneious Prime to trim the 3′ adapter sequence (TGGAATTCTCGGGTGCCAAGG) (SEQ ID NO: 58). The sequences were aligned to miR-125b ASO using Bowtie.

RNA Loading into RBCEVs

50 μg of RBCEVs were transfected with 1 μg of RNA using REG1 EV loading reagent (Carmine Therapeutics, USA) or an Exo-Fect™ Exosome Transfection Kit (System Biosciences, Canada) according to the manufacturer's protocols. Afterwards, free RNA and transfection reagents were washed away using centrifugation at 21,000×g for 30 min. For electroporation, 50 μg of RBCEVs were electroporated with 1 μg of RNA at 250V using a GenePulser Xcell electroporator (Bio-Rad) with exponential program at a fixed capacitance of 100 μF with 0.4 cm cuvettes.

RNA Loading into Lipid Nanoparticles (LNPs)

RNA-loaded LNPs were prepared by a dilution method. Briefly, 10 μg of NC-ASO, KRAS G12D ASO, immRNA, NC-ASO-immRNA, or KRAS G12D ASO-immRNA in 500 μl of HEPES buffer (10 mM, pH 4.0) was added to 500 μl of ethanol containing 185 nmole of DOTAP, 500 nmole of DOPE, 300 nmole of Cholesterol, and 15 nmole of DSPE-mPEG under vortexing. Then, 4.5 ml of HEPES (10 mM, pH 4.0) was added to the solution to dilute the concentration of ethanol. The resulting solutions were centrifuged (4000 g, 30 min, 20° C.) using Amicon Ultra 100K tube. The LNPs were next buffer-exchanged and concentrated by adding 4.0 ml of PBS pH 7.4 to the tubes and centrifuged again at 4000 g, 30 min, 20° C.

RNA Loading into Lipid Nanoparticles (LNPs)

1,2-dioleoyl-3-trimethylammonium-propane (chloride salt) (DOTAP), (1,2-Dioleoyl-sn-glycero-3-phosphoethanolamine (DOPE), 1,2-distearoyl-sn-glycero-3-phosphoethanolamine-N-[methoxy (polyethylene glycol)-2000] (ammonium salt)) (DSPE-mPEG2000), and Cholesterol were purchased Avanti Polar Lipids (Alabama, USA). RNA-loaded LNPs were prepared by dilution method. Briefly, 10 μg of NC-ASO, KRAS G12D ASO, immRNA, NC-ASO-immRNA, or KRAS G12D ASO-immRNA in 500 μl of HEPES buffer (10 mM, pH 4.0) was added to 500 μl of ethanol containing 185 nmole of DOTAP, 500 nmole of DOPE, 300 nmole of Cholesterol, and 15 nmole of DSPE-mPEG under vortexing. Then, 4.5 ml of HEPES (10 mM, pH 4.0) was added to the solution to dilute the concentration of ethanol. The resulting solutions were centrifuged (4000×g, 30 min, 20° C.) using Amicon Ultra 100K tube. The LNPs were next buffer-exchanged and concentrated by adding 4.0 ml of PBS pH 7.4 to the tubes and centrifuge again at 4000 g, 30 min, 20° C.

### Quantification of Loaded RNA in RBCEVs

Following loading with RNA, RBCEVs or LNPs were incubated with 1% Triton-X (Sigma) for 5 min at room temperature and then with heparin sulfate at a final concentration of 20 mg/mL for 1 hour at 37° C. After incubation, the mixture was loaded onto a 2% Tris-acetate-EDTA agarose gel with GelRed® nucleic acid gel stain (Sigma), separated at 100 V for 60 min and visualized with a ChemiDoc™ gel documentation system. The band fluorescence intensity was quantified using ImageJ v1.8.0.

Conjugation of RBCEVs with Anti-Human EGFR Nanobody

An RBCEV surface functionalization method was previously developed using OaAEP1 ligase to ligate peptides with ligase-binding motif (NGL) at the C-terminus onto RBCEV surface (Pham et al., 2021). For OaAEP1-mediated peptide ligation, RBCEVs were incubated for 3 hours in a solution with final concentration of 2 μM ligase and 500 μL biotinylated linker peptide (Biotin-TRNGL, GL Biochem, China) at 25° C. Following incubation, RBCEVs were washed three times with PBS using centrifugation at 21,000×g for 20 min. The biotin-TRNGL-ligated RBCEVs were then incubated with streptavidin (SA) (Abcam, UK) at a final concentration of 0.1 mg/mL for 2 hours at room temperature. The SA-biotin-RBCEVs were subsequently washed as described previously to remove free unbound streptavidin. For further functionalization, the sequences of anti-human EGFR nanobody (α-EGFR-VHH) and anti-mCherry nanobody (α-mCherry-VHH) were cloned with additional epitope tags for detection and purification. The VHH-coding DNA was synthesized and inserted into pET32(a+) plasmid, following a T7 promoter, by Guangzhou IGE Biotechnology Ltd (China). Nanobodies were expressed and purified as described previously (Pham et al., 2021). After purification, α-EGFR-VHH or α-mCherry-VHH was biotinylated using a Type B-Lightning-Link® Biotinylation Kit (Abcam) following the manufacturer's instructions. The biotin-hEGFR-VHH or biotin-mCherry-VHH was then incubated with SA-biotin-RBCEVs for 6 hours at 4° C. and washed twice with PBS. After washing, uncoated or VHH-coated RBCEVs were incubated with 20 μM CFSE (Life Technologies) for 1 hour at 37° C. and washed twice with PBS. After washing labelled RBCEVs were washed once using centrifugation and loaded into a size exclusion chromatographic (SEC) column (Izon, New Zealand) and eluted with PBS to wash away the unbound dye. RBCEVs, collected from SEC fraction 7 to 9, were washed twice with PBS by centrifugation at 21,000×g for 15 min at 4° C. For in vivo treatment, uncoated or VHH-coated RBCEVs were loaded with immRNA using REG1 as described earlier.

In Vitro Treatment of Cancer Cells with RBCEVs

A total of 50,000 4T1, CA1a or H358 cells were seeded in a 24-well plate prior to the incubation with 0.1 μg/μL immRNA-, 3p-125b-ASO-, 125b-ASO-, or NC-RNA-loaded RBCEVs for 24-72 hours in a humidified incubator at 37° C. with 5% CO2. Cells were then harvested for immunofluorescent imaging, RT-qPCR, or flow cytometry analysis. For dose response assay, 50,000 CA1a cells were incubated with 0.2, 0.1, 0.05, 0.025 and 0.0125 μg/μL of 125b-ASO-loaded RBCEVs for 24 hours and then collected for RT-qPCR analysis. For EV targeted delivery assay, 100,000 4T1-hEGFR cells were incubated with 0.02 μg/μL uncoated or VHH-coated CFSE-labelled or FAM-NC-ASO-loaded RBCEVs for 2 hours and then collected for flow cytometry analysis. 100,000 4T1-hEGFR cells were incubated with 0.1 μg/μL uncoated or VHH-coated immRNA-RBCEVs for 24 hours and then collected for RT-qPCR analysis.

To examine the knockdown efficacy of KRAS G12D, KRAS G12V, EGFR L858R, or EGFR T790M-targeting ASOs, 50,000 cells were seeded in each well of 12-well plates and cultured in a humidified incubator at 37° C. with 5% CO2 overnight. The cells were then treated with 100 nM NC ASO or oncogene-targeting ASO-loaded RBCEVs. After 48 hours, the cells were harvested for RT-qPCR and Western Blot analysis.

### Cell Viability Assay

A total of 10,000 CA1a cells were seeded in a 96-well plate prior to the incubation with 0.05 μg/μL NC-ASO-loaded RBCEVs. After 24, 48 and 72 hours, the cells were incubated with 10% (v/v) of CCK-8 reagent (Biosharp, China) for 2 hours at 37° C. with 5% CO2. The absorbance was measured at 450 nm using a microplate reader (Tecan, Switzerland).

To assess the anti-tumor effect of KRAS or EGFR ASO in combination with immRNA, 5,000 AsPC-1 cells were seeded in each well of a 96-well plate prior to the incubation with 0.1 μg/μl NC-ASO-, targeting-, immRNA-, NC-ASO-immRNA, or KRAS G12D ASO-immRNA-loaded RBCEVs or lipid nanoparticles for 48 hours. The cells were next incubated with 10% (v/v) of CCK-8 reagent and the absorbance was measured as described above. The cell viability was determined according to the following equation:

$$Cell viability ( \% ) = [ ( OD {450}_{( sample )} - OD {450}_{( blank )} ) / ( OD {450}_{( control )} - OD {450}_{( blank )} ) ] \times 100 \%$$

### Immunofluorescent Imaging

CA1a cells were pre-seeded on poly-D-lysine (Gibco, USA) coated 12 mm coverslips (Citoglass, China) 24 hours prior to treatment. Following treatment with FAM-NC-ASO-loaded RBCEVs, the coverslips were rinsed with fresh media, and stained with CellMask™ Deep Red Plasma Membrane Stains (Thermo Fisher Scientific) for 10 minutes at 37° C. Cell were rinsed twice in PBS and stained with Hoechst 33342 (Abcam) for 5 minutes at room temperature. The coverslips were rinsed three times with PBS before being fixed for 12 minutes using 4% paraformaldehyde (Alfa Aesar, USA) in PBS. The coverslips were subsequently washed three times with PBS followed by a final wash with distilled water before being mounted on slides using anti-fade fluorescence mounting medium (Abcam). Images were acquired using an Olympus FV3000 confocal microscope (Olympus, Japan). Image acquisition was conducted using Fluo View software while further analysis and quantification was conducted using ImageJ v1.8.0. Cell areas were selected as regions of interest (ROIs) based on the dilated mask of Hoechst signals. FAM signals were measured as mean pixel intensity of ROIs. Total measurement area covered 1200 to 1600 cells in each condition.

### IFN Reporter Assay

A549-Dual™ and A549-Dual™ RIG-I^{-/-} cells were seeded in 96-well plate at 10,000 cells per well in culture media for 18-24 hours before incubation with RBCEVs. 0.05 μg/μL immRNA-, 3p-125b-ASO-, or NC-RNA-loaded RBCEVs were incubated with the cells for 24 and 48 hours. Lucia luciferase in the supernatant was detected by the QUANTI-Luc reagent (InvivoGen) on a Synergy H1 microplate reader (Biotek, USA).

### Activation of Immune Cells in Co-Culture Systems

AsPC-1 cells were seed in a 12-well plate at a density of 50,000 cells/well and cultured in a humidified incubator at 37° C. with 5% CO2 overnight. The cells were then treated with RBCEVs delivered with 1 μg/ml of either NC ASO, KRAS G12D ASO, immRNA, or combination of KRAS G12D ASO and immRNA. After 48 hours, the cell culture media were harvested and centrifuged at 21,000×g for 20 minutes to completely remove free RBCEVs. The supernatants were collected and added to THP-1 cells pre-seeded in a 12-well plate at a density of 100,000 cells/well. After 24 hours, the treated THP-1 cells were collected, blocked with anti-human CD16/CD32 antibody, and stained with anti-human CD86-APC (Biolegend). The stained cells were then analyzed by FACS LSRII cytometer (BD BioSciences, USA).

Monocytes from Ficoll-isolated human peripheral blood mononuclear cells (PBMCs) were separated from lymphocytes by using human CD14 MicroBeads following the manufacture's protocol (Miltenyi Biotec, USA). The monocytes were differentiated into macrophages by culturing in RPMI medium supplemented with 10% fetal bovine serum, 1×penicillin-streptomycin, and 10 ng/ml M-CSF for 7 days prior to incubating with supernatants of treated PDOs. PCA067 PDOs were seeded in a 24-well plate until reaching a diameter of 50-200 nm. Thereafter, the PDOs were treated with RBCEVs delivered with 1 μg/ml of either NC ASO, KRAS G12D ASO, immRNA, or a combination of KRAS G12D ASO and immRNA. After 48 hours, the culture media were harvested and centrifuged at 21,000×g for 20 minutes to completely remove free RBCEVs. The collected supernatants were added to PBMC-derived macrophages. After 24 hours, the macrophages were harvested for RNA extraction and qPCR analysis

In Vivo Generation of Cancer Models and Treatment with RBCEVs

All mouse experiments were performed according to experimental protocols approved by the Institutional Animal Care and Use Committee of National University of Singapore. Mice of similar ages were tagged and grouped randomly for control and test treatments. Experiments were performed in a blinded manner. Exclusion was applied to the mice that accidentally died due to anaesthesia. BALB/c mice were purchased from In Vivos Pte Ltd, Singapore. NSG-SGM3 mice (NOD.CgPrkdc<scid>Il2rg<tm1Wjl>/Tg(CMV-IL3,CSF2,KITLG)1Eav/MloySzJ) and BALB/c nude mice (strain NU/JInv) were purchased from the Jackson Laboratory, USA.

### Intratumoral Administration of RBCEVs

Female 7-week-old BALB/c mice were injected with 1.25×10⁵ 4T1 cells in the 4^{th} mammary fat pad (MFP). After 10 days, mice were grouped randomly and injected with 2.5 mg/kg immRNA-loaded RBCEVs, 5 mg/kg 3p-125b-ASO-loaded RBCEVs, or same amount of NC-RNA-loaded RBCEVs as controls intratumorally. Intratumoral injection was repeated every three days, five times in total. For PD-L1 blockade treatment, the mice bearing 4T1 tumours were injected intraperitoneally with 2 mg/kg of anti-mouse PD-L1 monoclonal antibody (BioXCell, USA) one day after intratumoral injection of immRNA-loaded RBCEVs. The treatment was repeated five times at intervals of three days. Tumour size was measured every two days using digital calipers. Mice were sacrificed when untreated tumours approached ˜15 mm in diameter. Tumours were collected for RNA extraction, flow cytometry analysis or imaging.

Female 7-week-old NSGS mice were injected with 1×10⁶ CA1a cells in the 4^{th} mammary fat pad (MFP). After fourteen days, mice were grouped randomly and injected with 2.5 mg/kg immRNA- or NC-RNA-loaded RBCEVs intratumorally. Intratumoral injection was repeated every three days, five times in total. Tumour size was measured every two days using digital calipers. Mice were sacrificed when untreated tumours approached ˜15 mm in diameter. Tumours were collected for RNA extraction, flow cytometry analysis or imaging.

Female 7-week-old BALB/c mice were subcutaneously injected with 5×10⁵ CT26 cells on the right flank. When the tumor volume reached around 40-50 mm³, the mice with similar tumor volumes and body weight were selected and randomly divided into four groups (denoted as day 0) and intratumorally injected with 2.5 mg/kg RBCEVs, NC ASO, KRAS G12D ASO, or 5 mg/kg KRAS G12D ASO and immRNA-loaded RBCEVs at a 2-days interval. Tumor size was measured every two days using digital calipers. Mice were sacrificed when untreated tumors approached ˜15 mm in diameter. The isolated tumors were measured regarding absolute wet-weights by an automatic electronic balance. Tumors were then subjected to RNA extraction or imaging.

### Intrapulmonary Targeted Delivery of RBCEVs

To generate a breast cancer metastasis model, 4T1 cells were transduced with a lentiviral vector (pHAGE-EGFR, Addgene, USA), selected with puromycin (Santa Cruz) and sorted using Aria II sorter (BD Biosciences) to create a stable cell line with high expression level of human EGFR. A total of 2.5×10⁵ 4T1-hEGFR cells were injected intravenously in female 7-week-old BALB/c mice. One day post inoculation, intrapulmonary delivery of 25 mg/kg uncoated or VHH-coated immRNA-RBCEVs was conducted every two days in the respective mice using a mouse Microspray Aerosolizer (Yuyan, China). After five treatments, the mice were sacrificed and the lungs were excised for RNA extraction, flow cytometry analysis or imaging. For intrapulmonary biodistribution of RBCEVs, 5×10⁵ 4T1-hEGFR cells were injected intravenously in female 7-week-old BALB/c mice. After seven days, intrapulmonary delivery of 25 mg/kg uncoated or VHH-coated CFSE-RBCEVs was carried out. After 24 hours, the mice were sacrificed and the lungs were excised for flow cytometry analysis.

To generate an orthotopic lung cancer model, H1975 cells were transduced with a lentiviral vector (pLenti-mCherry-luc, Addgene, USA), selected with puromycin (Santa Cruz) and sorted using Aria II sorter (BD Biosciences) to create a stable luciferase and mCherry-expressing H1975 cell line (Luc-mCherry-H1975). A total of 1×10⁶ Luc-mCherry-H1975 cells were injected intravenously in 8-week-old NSGS mice. After five weeks, the mice were subjected to IVIS imaging to check the tumor growth at lung. The mice with similar bioluminescence intensity were divided into two groups (denoted as day 0) and intratracheally administrated with 5 mg/kg NC ASO or EGFR L858R ASO-loaded RBCEVs at a 3-days interval. On day 14, mice were sacrificed, and lung tissues were collected for H&E and immunohistochemistry staining.

### Flow Cytometry Analysis

### RBCEV Analysis

To quantify FAM-NC-ASO loading efficiency, 50 μg of RBCEVs were incubated with 2.5 μg of latex beads (Thermo Fisher Scientific) overnight at 4° C. on a shaker, washed three times with PBS and resuspended in 100 μL of FACS buffer. FACS analysis of latex beads was performed using a CytoFlex LX cytometer (Beckman Coulter). FACS plots were generated using FlowJo v10.0.7.

### Apoptosis Analysis

Following treatment with RBCEVs, cells were collected and washed with PBS. Apoptosis was determined by Annexin V (ANXV)/propidium iodide (PI) staining with the apoptosis detection kit (Life Technologies). Briefly, 50,000 treated cells were incubated with ANXV and PI in binding buffer for 15 min at 4° C. The cells were then analyzed by FACS LSRII cytometer (BD BioSciences, USA) or CytoFlexS cytometer (Beckman Coulter). FACS plots were generated using FlowJo v10.0.7.

### Immune Cell Analysis

Tumours were excised, washed with PBS and dissociated in DMEM media containing 10% FBS and 5 mg/mL collagenase IV (Thermo Fisher Scientific) using the GentleMACS dissociator (Miltenyi Biotech, Germany). Cells were filtered through a 70 μm strainer, blocked with anti-mouse CD16/CD32 antibody (BioLegend), and stained with anti-mouse CD45-PECy7 (BioLegend), anti-mouse CD11b-FITC (BioLegend), anti-mouse F4/80-APC (BioLegend), anti-mouse Ly6G/C-APC (BioLegend), anti-mouse CD3ε-APC (BioLegend), anti-mouse CD11c-PB (BioLegend), anti-mouse CD49b-APC (BioLegend), anti-mouse SiglecF-PE (BioLegend), anti-mouse CD103-APC (BioLegend), anti-mouse CD4-BV421 (BioLegend), anti-mouse CD8-APC (BioLegend), anti-mouse MHCII-PE-Cy7 (BioLegend), anti-mouse CD206-APC (BioLegend), anti-mouse CD86-APC-Cy7 (BioLegend), anti-FLAG-APC antibody (BioLegend) or anti-human EGFR nanobody for 30 min at 4° C. Cells were subsequently washed three times in FACS buffer. Cells were analyzed using a FACS LSRII cytometer (BD BioSciences) or CytoFLEX S cytometer (Beckman Coulter) or CytoFLEX LX cytometer (Beckman Coulter). Dead cells were identified by staining with SYTOX™ dye (Thermo Fisher Scientific) and removed from the analysis. FCS files were analyzed using FlowJo v10.0.7, Kaluza (Beckman Coulter) or Cytobank viSNE (Beckman Coulter). Briefly, cells were first gated using an FSC-A versus SSC-A plot to exclude debris and dead cells. Single cells were subsequently gated via an FSC-W versus FSC-H plot, excluding doublets and aggregated cells. The fluorescent-positive population of cells was subsequently gated by targeted fluorescent channels and then subjected to viSNE analysis. Equal event sampling was selected. viSNE plots for each individual parameter were downloaded from Cytobank. Cellular phenotypes were assigned to the viSNE plot based on distribution and expression characteristics using phenotypic markers.

### Ex Vivo Analysis of Tumour-Specific T Cell Activity

For detection of CD69 and granzyme B expression of tumour-infiltrated CD8+ T cells, CD8+ T cells from dissociated tumour cells were selected using CD8+ tumour-infiltrating lymphocytes isolation kit (Miltenyi Biotec). The isolated CD8+ T cells were blocked with anti-mouse CD16/CD32 antibody and stained with anti-mouse CD69-PE (BioLegend) or anti-mouse granzyme B-FITC antibody (BioLegend). Data were obtained using a FACS LSRII cytometer and analyzed using FlowJo. For detection of tumour-specific T cell activity, isolated CD8+ T cells were incubated with 4T1-hEGFR-luciferase cells at a ratio of 50:1 for 48 h. The supernatant devoid of floating cells was harvested. The quantity of IFNγ of each supernatant was analyzed using a mouse IFNγ ELISA kit (BioLegend) according to the manufacturer's instruction. The tumour cell viability was assessed using a luciferase assay kit (Promega, USA) according to the manufacturer's instruction. Data were obtained using a microplate reader (Tecan).

### Mouse Cytokine Immunoassay

Mouse blood was harvested by cardiac puncture at the endpoint of in vivo treatments. The whole blood was clotted for 30 min at 37° C. and the sera were isolated following centrifugation at 5,000 rpm for 20 min. The isolated sera were collected and stored at −80° C. The concentration of IFNβ in the sera was measured using a Recombinant Mouse IFNβ ELISA kit (Biolegend) according to the manufacturer's instruction. For multiplex immunoassay, cell culture supernatant was collected following centrifugation at 300×g for 5 min. Tumours were excised and homogenized in cold RIPA buffer supplemented with protease inhibitor. Tumour lysates were collected using centrifugation at 12,000×g for 20 min. The concentrations of cytokines in the collected supernatant or tumour lysates were measured using a ProcartaPlex™ Multiplex Immunoassay kit (Invitrogen, USA) according to the manufacturer's instruction. The cytokines assayed include IFNα, IFNβ, IL-6, IL-10, IL-12p40 and TNFα.

### RNA Extraction and RT-qPCR

Total RNA was extracted from cells or tissues using TRIzol (Thermo Fisher Scientific) according to the manufacturer's manuals. RNA was converted to cDNA using a high capacity cDNA reverse transcription kit (Thermo Fisher Scientific) following the manufacturer's protocol. mRNA levels were quantified using Ssofast® Green qPCR kit (Bio-Rad), normalized to GAPDH (for primer sequences, see Supplementary Table S1). miRNA levels were quantified using Taqman® miRNA qPCR kit (Thermo Fisher Scientific), normalized to snoRNA234 (mouse) or U6B snRNA (human). All qPCR reactions were performed using a CFX96 Touch™ Real-Time PCR Detection System (Bio-Rad) or a QuantStudio 6 Flex Real-Time PCR System (Life Technologies).

### Hematoxylin and Eosin (H & E) Staining and TUNEL Assay

Following overnight fixation in 10% neutral buffered formalin (Sigma), tumor or lung tissues were sequentially dehydrated in 70%, 80%, 90% and 100% ethanol at 37° C. using a Leica TP1020 tissue processor (Leica, Germany). Samples were cleared in three baths of Histo-Clear (National Diagnostics, USA), each for 1.5 hours at 37° C., and impregnated in 3 baths of paraffin wax (Thermo Fisher Scientific), each for 1 hour at 62° C., respectively. The paraffin blocks were sectioned at 5 μm using a Leica RM2255 rotary microtome. Sections were dried at 37° C. and serially dewaxed in 3 baths of Histo-Clear, then immersed in 2 baths of absolute ethanol and 1 bath of 70% ethanol, each for 10 min. Sections were rehydrated in 90%, 75% and 50% ethanol, each for 5 min, and distilled water for 10 min. Subsequently, sections were stained with Hematoxylin (Abcam) for 5 min. After washing with water, the sections were treated with 0.3% acid alcohol, washed and blued with bluing reagent (Abcam). Sections were subsequently stained with 0.5% Eosin (Abcam) for 1 min. After washing with water, sections were dehydrated in absolute ethanol, cleared in Histo-Clear then mounted using Histomount mounting solution (National Diagnostics). Images were acquired with a TissueFAXS PLUS slide scanner (TissueGnostics, Austria). Image acquisition was conducted using TissueFAXS viewer software while further analysis and quantification was conducted using ImageJ v1.8.0.

Apoptosis was evaluated using a TUNEL assay BrdU-Red kit (Abcam) in combination with immunofluorescent staining. 4T1 tumour sections were washed twice with PBS. Retrieval of antigen was conducted by superheating the sections in a microwave oven for 15 min until boiling of antigen retrieval solution (Tris-EDTA, pH 9.0) was attained. Sections were allowed to cool down in the retrieval solution for 30 min. Blocking buffer (5% normal donkey serum and 0.3% TritonX-100 in PBS) was applied for 1 h at room temperature. After incubation with rabbit anti-mouse α-smooth muscle actin antibody (Abcam, dilution 1:250) overnight at 4° C., the sections were incubated with donkey anti-rabbit secondary antibody conjugated with Alexa Fluor® 647 fluorophore (Jackson ImmunoResearch, dilution 1:200) for 1 h at room temperature. Sections were then stained using the TUNEL assay kit according to the manufacturer's protocol. After staining, images of TUNEL staining were acquired with an LSM-710 NLO confocal microscope (Zeiss, Germany) or an Olympus FV3000 confocal microscope (Olympus). Image acquisition was conducted using Zeiss Zen software or FluoView software while further analysis and quantification was conducted using ImageJ v1.8.0. Nuclei areas were selected as regions of interest (ROI) based on Hoechst signal. BrdU-Red signals were measured as mean pixel intensity in selected ROIs.

### Statistical Analysis

All statistical analysis was performed using Student's two-tailed t-tests in GraphPad Prism 8 (GraphPad Software, CA) to determine significant differences between treated samples and control. P-values less than 0.05 were considered significant, based on at least 3 independent replicates. In all the graphs, data are presented as median or mean and standard error of the mean (SEM). Animal experiments were repeated in groups of 5 to 6 mice. The minimum sample size of 3 was determined using G*Power analysis which compares the mean difference of 2 independent groups with a error prob=0.05, effect size d=5 and power=0.95.

### Results

RBCEVs can be Loaded with Small RNAs Using Multiple Methods for the Delivery of RNAs to Cancer Cells

RBCEVs were purified according to a previous protocol (Usman et al., 2018). Purified RBCEVs were enriched in common EV markers, such as ALIX and TSG101, as well as hemoglobin A (HBA), the major RBC protein (FIG. 11A). It was also found that RBCEVs express glycophorin A (GPA) on the surface as an exclusive marker for EVs originated from human RBCs (FIGS. 11A-B). The cytoskeleton protein β-actin was almost absent in RBCEVs, suggesting that purified RBCEVs did not contain cellular debris (FIG. 11A). Additionally, purified RBCEVs were homogenous in size, 120-200 nm in diameter, as determined using a nanoparticle analyzer (FIG. 1D).

In order to deliver therapeutic RNAs to cancer cells, RBCEVs were loaded with small RNAs using multiple methods including electroporation, Exo-Fect-mediated transfection and REG1-mediated transfection. RBCEVs were separated from RNA-transfectant complexes using three rounds of centrifugation. Bead-assisted flow cytometry analysis of RBCEVs loaded with FAM-ASO revealed that FAM-ASO was loaded efficiently into RBCEVs using three different methods (FIGS. 1A-B). The loading efficiency by Exo-Fect and REG1 were relatively higher than that by electroporation (FIGS. 1A-B). It was also observed that the loading with REG1 yielded a homogenous population of RBCEVs with strong FAM fluorescence (FIGS. 1A-B). In contrast, a simple incubation did not facilitate the entry of FAM-ASO into RBCEVs (FIG. 1A). Transmission electron microscopy revealed that following REG1 transfection, RBCEVs retained a similar morphology to untreated control, while the electroporated and Exo-fect-transfected RBCEVs showed signs of aggregation as indicated by the greater extent of ruffled membranes (FIG. 1C). Nanoparticle tracking analysis also revealed that REG1 maintained the size of RBCEVs (FIG. 1D). Upon electroporation and Exo-Fect transfection, RBCEVs were prone to aggregation as evidenced by the increase in the proportion of EVs with larger particle size, ˜400-600 nm in diameter (FIG. 1D).

The uptake of RBCEVs loaded with FAM-ASO was further investigated using three loading methods by human breast cancer MCF10aCA1a (CA1a) cells. FACS and immunofluorescent analysis demonstrated that CA1a cells readily took up RBCEVs containing FAM-ASO (FIGS. 1E-G). Confocal imaging revealed that the FAM signal was present inside the cells (FIG. 1E). Exo-Fect-transfected RBCEVs exhibited the highest uptake by CA1a cells shown by FACS (FIG. 1G), whereas REG1-transfected RBCEVs were taken up the most by CA1a cells shown by immunofluorescent analysis (FIGS. 1E-F). To evaluate the potential toxic effects of three loading methods, CA1a cells were incubated with electroporated, Exo-Fect-transfected and REG1-transfected RBCEVs for three days. CCK-8 assay analysis showed that electroporation and REG1 were non-toxic to CA1a cells, whereas Exo-fect exhibited a mild toxicity to the cells after 24 h incubation but not at later time points (FIG. 1H). Overall, RBCEVs treated with REG1 and Exo-Fect are comparable in delivery. Both REG1 and Exo-Fect perform better than electroporation for the loading of RNA into RBCEVs. As such, Exo-Fect and REG1 were used as transfectants for RBCEVs in the subsequent experiments. To estimate the loading efficiency of the transfectants, RBCEVs were transfected with miR-125b ASO using Exo-Fect and REG1 and then lysed by detergents. miR-125b ASOs were separated by gel electrophoresis (FIG. 1I). Based on the band fluorescent intensity, approximately ˜94.2% and ˜86% of 125b-ASO was observed to be loaded in RBCEVs by Exo-Fect and REG1, respectively (FIG. 1I). Of the two transfection methods, REG1 conferred stronger inhibition of miR-125b in CA1a cells with similar transfection efficiency as shown by a dose response assay (FIG. 1J).

### RBCEVs Deliver Immunomodulatory RNA to Activate the RIG-I Pathway and Induce Immunogenic Cell Death of Cancer Cells

A series of immRNA species are known in the art as potent RIG-I agonists in human skin dendritic cells and macrophages (Yong et al., 2019; Ho et al., 2019). Among the immRNA variants, 3p10LA9 showed the strongest cellular IFNs-producing activity. To examine the effects of the immRNA 3p10LA9 (FIG. 2A) on breast cancer cells, RBCEVs were transfected with immRNA using REG1 and incubated the immRNA-loaded RBCEVs with the highly metastatic mouse breast cancer 4T1 cells for 24 hours. A scrambled RNA was used as a negative control (NC). qPCR analysis revealed that the uptake of immRNA-loaded RBCEVs by 4T1 cells led to significant up-regulation of cellular RLRs, Ddx58 (the gene encoding RIG-I) and Mda5, as well as RLR downstream effectors including Irf3, Irf7, Ifnb, Rsad2 (Viperin) and Isg56, as compared to NC-RNA-loaded RBCEVs (FIG. 2B). The effects of immRNA were also assessed in human triple-negative basal-like breast cancer (TNBC) CA1a cells, which is a progressively aggressive and metastatic derivative of MCF10A cells, with the same doses of immRNA-loaded RBCEVs, yielding a similar up-regulation of RIG-I related genes. The uptake of immRNA-loaded RBCEVs by CA1a cells up-regulated the cellular levels of DDX58, MDA5, IRF3, IRF7, IFNB, RSAD2 and ISG56 significantly (FIG. 2C). Similarly, human TNBC MDA-MB-468 cells were highly responsive to immRNA-loaded RBCEVs as shown by substantial up-regulation of DDX58, MDA5, IRF7, IFNB, RSAD2, and ISG56 (FIG. 12A). However, immRNA-loaded RBCEVs did not induce RIG-I activation in human TNBC MDA-MB-231 cells (FIG. 12B).

The delivery of immRNA to lung cancer cells was also tested. Human lung cancer NCI-H358 (H358) cells showed significant up-regulation of DDX58, MDA5, MAVS, IRF7, IFNB, RSAD2, and ISG56, after incubation with immRNA-loaded RBCEVs (FIG. 2D). RSAD2, known as an IFN-stimulated gene, increased by 305-fold, 518-fold and 143-fold, respectively, in all three cell lines (FIGS. 2B-D and 12A).

To assess the effects of immRNA on normal cells, untransformed mammary gland epithelial MCF10A cells were incubated with immRNA-loaded RBCEVs, which led to a slight increase in the level of DDX58 and RSAD2 but not other RIG-I-related genes in the cells (FIG. 12C). Mouse lung epithelial (mLE) cells were also produced using a previously established protocol (Kasinski & Slack, 2013). Surface marker EpCAM of the cells was determined by flow cytometry (FIG. 11C). The uptake of immRNA-loaded RBCEVs by lung epithelial cells led to significant up-regulation of Ddx58, Mda5, Irf7, Rsad2, and Isg56. Ifnb was not detectable in the cells, whereas Ifna4, Ifna11, and Ifna12 were upregulated significantly (FIG. 12D).

Type-I-IFNs-producing activity induced by immRNA was assessed. immRNA-loaded RBCEVs were incubated with human lung epithelial carcinoma A549-Dual™ and A549-Dual™ RIG-I^{-/-} cells. These two cell lines secrete the interferon regulatory factor (IRF) pathway mediated Lucia luciferase under the control of an ISG54 minimal promoter in conjunction with five IFN-stimulated response elements. As a result, the wild-type (WT) A549-Dual™ cells incubated with immRNA-RBCEVs were responsive to this RIG-I activator as shown by the increased luciferase activity after 24 and 48 hours (FIG. 2E). Removal of the RIG-I gene from the cells resulted in complete abrogation of IFN signalling in response to immRNA (FIG. 2E), confirming RIG-I as the primary immRNA sensor in the cells. To evaluate the effect of immRNA on the inflammatory response, the concentrations of key inflammatory mediators were measured in 4T1-conditioned media. 4T1 cells treated with immRNA-loaded RBCEVs secreted substantially higher levels of IFNα (˜55.95 pg/mL) and IFNβ (˜1844.33 pg/mL) (FIG. 2F). Furthermore, immRNA-RBCEVs taken up by 4T1 cells also induced the secretion of proinflammatory cytokines TNFα (˜10.91 pg/mL) and IL-6 (˜793.22 pg/mL) (FIG. 2F).

The induction of immunogenic cell death in cancer cells was subsequently quantified using Annexin V (ANXV) and Propidium Iodide (PI) staining for each EV treatment. After 72 h of incubation, immRNA-loaded RBCEVs induced apoptosis of 4T1 cells (˜15.9%) (FIGS. 2G and 12E), CA1a cells (˜24.6%) (FIGS. 2H and 12E), H358 cells (˜17.6%) (FIGS. 2I and 12E), and MDA-MB-468 cells (˜16.0%) (FIGS. 12E-F) as shown by the average percentage of ANXV+PI+ population. ImmRNA-loaded RBCEVs failed to induce cell death in RIG-I-low MDA-MB-231 cells (FIGS. 12E-F), indicating that RIG-I is an essential receptor in response to immRNA. MCF10A cells and mLE cells did not succumb to apoptosis after the incubation with immRNA-loaded RBCEVs (FIGS. 12E-F). These data suggest that immRNA-loaded RBCEVs activate the cellular RIG-I pathway, triggering type I IFN secretion and inducing cell death in cancer cells, while concurrently sparing non-malignant cells.

RBCEVs Deliver Bi-Functional ASOs to Simultaneously Inhibit Oncogenic miR-125b and Activate the RIG-I Pathway Leading to Cell Death in Cancer Cells

After confirming that 5′ triphosphate immRNA could effectively trigger RIG-I activation, a combinatorial approach that incorporates ASO-mediated oncogene silencing and RIG-I-mediated immune activation simultaneously was tested. A miR-125b ASO coupled with a triphosphate group at the 5′ end (3p-125b-ASO) (FIG. 3A) was designed. To improve the yield of the 3p-125b-ASO, the first four nucleotides of the original 125b-ASO (full-length complementary to miR-125b) were removed and replaced with two guanine nucleotides. A couple of 3′ end truncations were also tested. A dsDNA template of the modified 125b-ASO sequence was inserted after the T7 promoter for efficient in vitro transcription (IVT) according to an established protocol (Luo et al., 2012; Yong et al., 2019). This resulted in a longer product due to the product RNA rebound to the T7 RNA polymerase and self-primed (in cis) generation of a hairpin duplex (FIG. 13A). The sequence of the IVT-produced 3p-125b-ASO was confirmed by sequencing.

To assess the functional impacts of 3p-125b-ASO in cancer cells, 3p-125b-ASO was delivered using RBCEVs. Following incubation of ASO-loaded RBCEVs with cancer cells for 24 hours, the miR-125b silencing activity of the IVT-produced 3p-125b-ASO was compared with an unmodified miR-125 ASO (125b-ASO) carrying a free 5′-OH group using qPCR. As a result, unmodified 125b-ASO and modified 3p-125b-ASO inhibited endogenous miR-125b levels to a similar extent in 4T1 cells, CA1a cells and H358 cells (FIGS. 3B, D, F). Thus, silencing activity was not impeded by the presence of the triphosphate group at the 5′ end. The RIG-I signalling in cancer cells was then assessed in response to the treatment with 3p-125b-ASO-loaded RBCEVs. The uptake of 3p-125b-ASO-loaded RBCEVs induced significant up-regulation of Ddx58, Mda5, Irf7, Ifnb, Rsad2 and Isg56 in 4T1 cells (FIG. 3C); DDX58, MDA5, MAVS, IRF3, IRF7, IFNB, RSAD2 and ISG56 in CA1a cells (FIG. 3E); DDX58, MDA5, MAVS, IRF7, IFNB, RSAD2 and ISG56 in H358 cells (FIG. 3G). Silencing of miR-125b by the unmodified 125b-ASO had no effects on RIG-I activation in the three cell lines (FIGS. 3C, E, G). Similar to immRNA-loaded RBCEVs, 3p-125b-ASO-loaded RBCEVs showed no effect on MDA-MB-231 cells, modest effects on MCF10A cells, and up-regulated Ifna but not Ifnb via RIG-I activation in mLE cells (FIGS. 13B-D).

To validate the activity of type-I IFNs secretion induced by 3p-125b-ASO, the reporter assay was repeated using A549-Dual™ and A549-Dual™ RIG-I^{-/-} cells. RIG-I stimulation by 3p-125b-ASO-loaded RBCEVs significantly induced the secretion of IFNs in A549-Dual™ wild type (WT) cells after 24 and 48 hours in comparison with A549-Dual™ RIG-I^{-/-} cells (FIG. 3H). RIG-I knockout confirmed the critical role of RIG-I for inducing IFN secretion. Likewise, the cytokine immunoassay analysis revealed that 4T1 cells taking up 3p-125b-ASO-loaded RBCEVs released significantly higher levels of IFNα (˜25.42 pg/mL), IFNβ (˜815.41 pg/mL) and TNFα (˜8.23 pg/mL) (FIG. 3I).

To examine the functional impact of 3p-125b-ASO on intrinsic apoptosis in cancer cells, cancer cells were incubated with 3p-125b-ASO-RBCEVs for 72 h. The treatment with 3p-125b-ASO-RBCEVs strongly induced apoptosis in 4T1 (˜15.5%) (FIGS. 3J and 13E), CA1a (˜21.7%) (FIGS. 3K and 13E), and H358 cells (˜14.6%) (FIGS. 3L and 13E), similar to the effect of the original 125b-ASO, determined by ANXV and PI binding. 3p-125b-ASO-RBCEVs also induced cell death in MDA-MB-231 cells, and exerted modest cytotoxicity to non-malignant cells, which is an effect attributed to the inhibition of miR-125b in the cells (FIGS. 13E-F). Of note, the combinatorial treatments of immRNA-RBCEVs and 3p-125b-ASORBCEVs in 4T1 cells and CA1a cells did not enhance the effect of each agent on RIG-I activation and apoptosis (FIGS. 13G-H). In contrast, the combination of immRNA-RBCEVs and 3p-125b-ASO-RBCEVs resulted in less activity of RIG-I response pathway and exhibited less effect on apoptosis than single-agent treatments (FIGS. 13G-H). Together, these data suggest that the bi-functional 3p-125b-ASO comprises two distinct and independent functions, which can simultaneously inhibit oncogenic miR-125b and activate RIG-I signalling, leading to immunogenic death of cancer cells.

Intratumoral Delivery of immRNA Suppresses Breast Cancer Growth by Triggering RIG-I Mediated Immune Responses

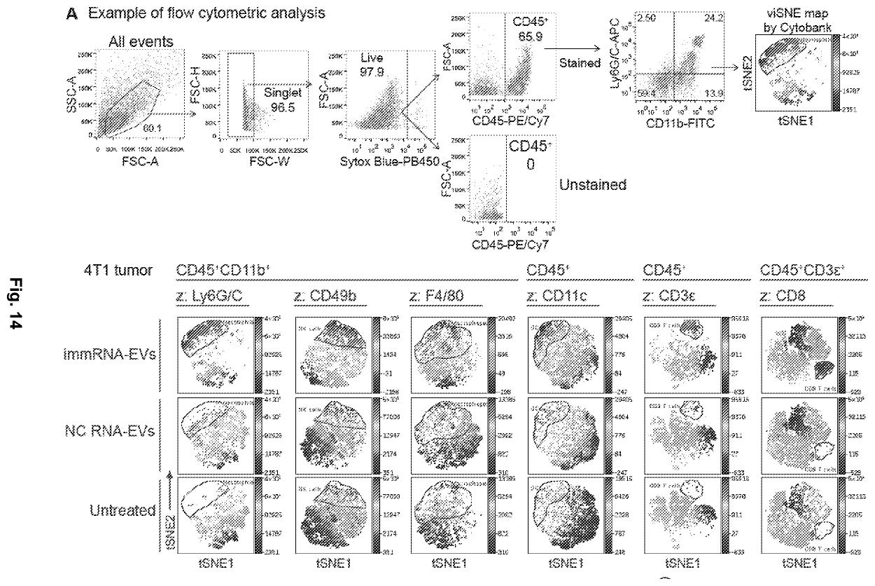

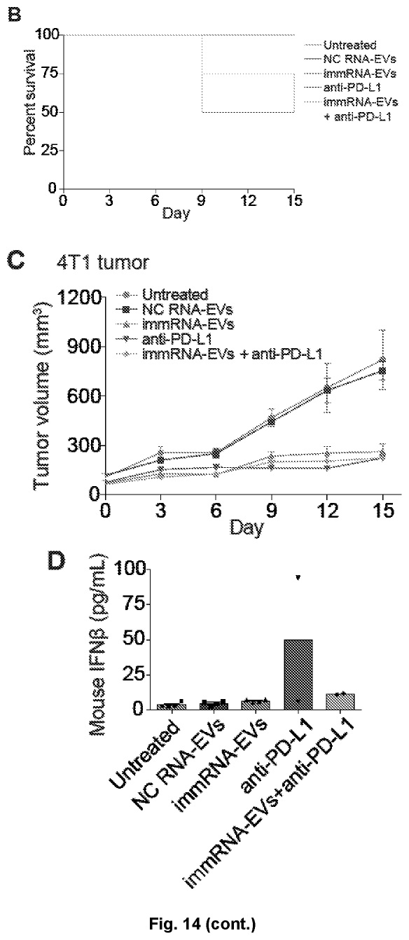

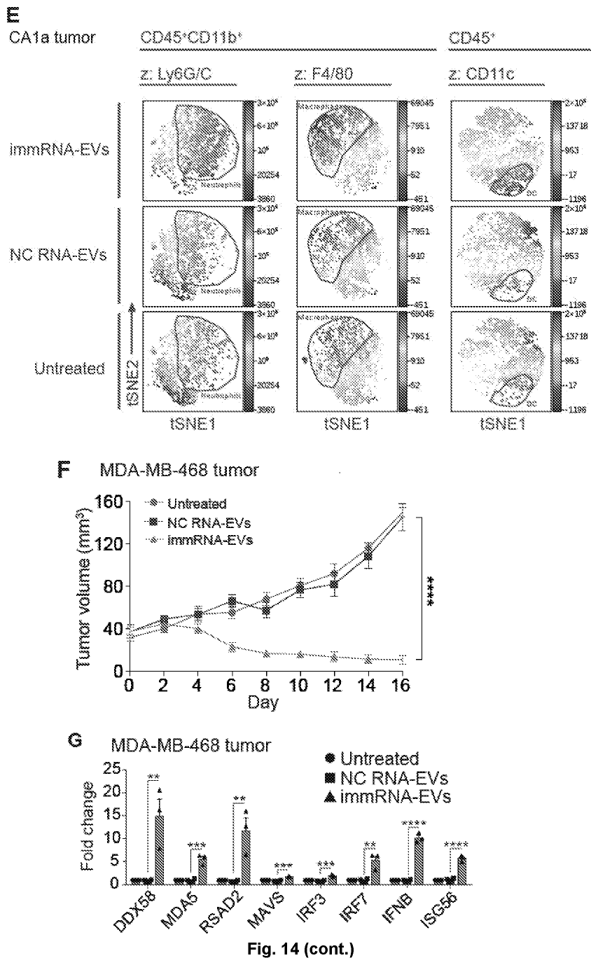

To examine the anti-tumour activity of immRNA in vivo, 4T1 cells were implanted in the 4th mammary fat pad (MFP) of BALB/c mice (FIG. 4A). Tumours were treated with RBCEVs containing immRNA or NC RNA intratumorally every three days and monitored the growth of tumours (FIG. 4A). The treatment with immRNA-loaded RBCEVs was observed to significantly dampened 4T1 tumour growth as compared to NC-RNA-loaded RBCEVs (FIG. 4B). The mice were sacrificed and tumours were excised on day 18 when the untreated tumours reached ˜15 mm in diameter. qPCR analysis of the dissociated tumour cells revealed that the delivery of immRNA-loaded RBCEVs led to up-regulations of Ddx58, Mda5, Mavs, Irf3, Irf7, Ifnb, Rsad2 and Isg56 in the tumors (FIG. 4C). To confirm the cytotoxic effects of immRNAloaded RBCEVs on the tumours, TUNEL staining was conducted, and the tumour sections were co-stained with an antibody for α-smooth muscle actin (αSMA) to mark cancer-associated fibroblasts, the most abundant cell type in the tumour stroma. Increased apoptosis was observed in tumours treated with immRNA-loaded RBCEVs, compared to the treatment with NC-RNA-loaded RBCEVs (FIG. 4G-H). Furthermore, TUNEL positive cells gathered at the tumour bulk rather than within the stromal compartment (FIG. 4G). Interestingly, a potent immune activation was observed as evidenced by increased infiltration of neutrophils (CD45+CD11b+Ly6G/C+), natural killer (NK) cells (CD45+CD11b+CD49b+), macrophages (CD45+CD11b+F4/80+), dendritic cells (DCs) (CD45+CD11c+), total T cells (CD45+CD3ε+), and CD8+ T cells (CD45+CD38+CD8+) in the tumours treated with immRNA-RBCEVs in comparison with NC-RNA-loaded RBCEVs (FIGS. 4D and 14A). Additionally, the serum IFN-β level was measured at the endpoint. Intratumoral administration of immRNA-loaded RBCEVs slightly increased serum IFN-β level (˜6.99 pg/mL) in mice (FIG. 4E). To further characterize the cytokine milieu in the tumours, qPCR of cytokine-encoding genes was conducted in the dissociated tumour cells. The results revealed that immRNA-loaded RBCEVs significantly increased the mRNA levels of Ifna4, Ifna11, and Ifna12, and multiple pro-inflammatory cytokine genes, including Tnf, Il6, Il12b, 112, Ifng, Il1b, and Il18, while decreasing the levels of anti-inflammatory cytokine genes, including Il4, Il5 and Tgfb1, indicative of a shift from Th2 toward Th1 immune response (FIG. 4F).

4T1 tumours were treated with 2.5 mg/kg immRNA-loaded RBCEVs (intratumoral (i.t)) and 2 mg/kg anti-PD-L1 monoclonal antibody (intraperitoneal (i.p.)) one day apart. Monotherapy of immRNA-loaded RBCEVs or anti-PD-L1 antibody was used as a control. The dose and frequency of anti-PD-L1 antibody treatment was applied based on a previous study (Hong et al., 2019). 2 mg/kg of anti-PD-L1 is 34 times lower than the clinical-equivalent dose (Herbst et al., 2014). Surprisingly, it was observed that 2 out of 4 mice treated with anti-PD-L1 alone and 2 out of 4 mice treated with immRNARBCEVs combined with anti-PD-L1 were dead after three or five doses (FIG. 14B). In contrast, all of the mice survived after five doses of immRNA-RBCEVs monotherapy treatment (FIG. 14B). The serum IFNβ level of the survived mice at the endpoint indicated that anti-PD-L1 alone triggered cytokine release syndrome, which seemed to be alleviated when anti-PD-L1 was combined with immRNA-RBCEVs even though the endpoint-related mortality was the same as that caused by anti-PD-L1 monotherapy (FIG. 14D). The combined treatment and anti-PD-L1 treatment alone did not have better effects on tumour suppression as compared to immRNA-RBCEVs monotherapy (FIG. 14C). Therefore, these data suggest that immRNA-loaded RBCEVs are safe and sufficiently effective as a single-agent immunotherapy for breast cancer treatment.

As a model of human breast cancer, CA1a cells were implanted in the fourth MFP of immunocompromised NSG-SGM3 mice (FIG. 4I). Similarly, the tumours were treated with immRNA-loaded RBCEVs intratumorally every three days and measured the tumour size (FIG. 4I). The treatment with immRNA-loaded RBCEVs inhibited the growth of CA1a tumours significantly (FIG. 4J). qPCR results showed that the RIG-I cascade including DDX58, MDA5, MAVS, IRF3, IRF7, IFNB, RSAD2, and ISG56 was up-regulated in the tumours upon immRNA-RBCEVs treatment (FIG. 4K). Additionally, the treatment with immRNA-RBCEVs was associated with enhanced infiltration of neutrophils, macrophages and dendritic cells in the tumours (FIGS. 4L and 14E). The same set of experiments was carried out in the MDA-MB-468 tumour model in nude mice, with four mice per group. immRNA-loaded RBCEVs treatment led to rapid and extensive shrinkage of MDA-MB-468 tumours (FIG. 14F), one of which was eliminated. RIG-I activation was addressed with the limited number of tumour cells by qPCR. The results revealed that immRNA-loaded RBCEVs treatment activated the RIG-I pathway by up-regulating DDX58, MDA5, RSAD2, MAVS, IRF3, IRF7, IFNB, and ISG56 in the tumour cells (FIG. 14G). Taken together, these data suggest that intratumoral administration of immRNA-RBCEVs triggers anti-tumour responses by activating the RIG-I pathway and recruiting immune cells for orthotopic and xenograft breast cancer suppression.

### Intratumoral Delivery of Bi-Functional ASO Suppresses Breast Cancer Growth by Triggering Apoptosis and RIG-I Mediated Immune Responses

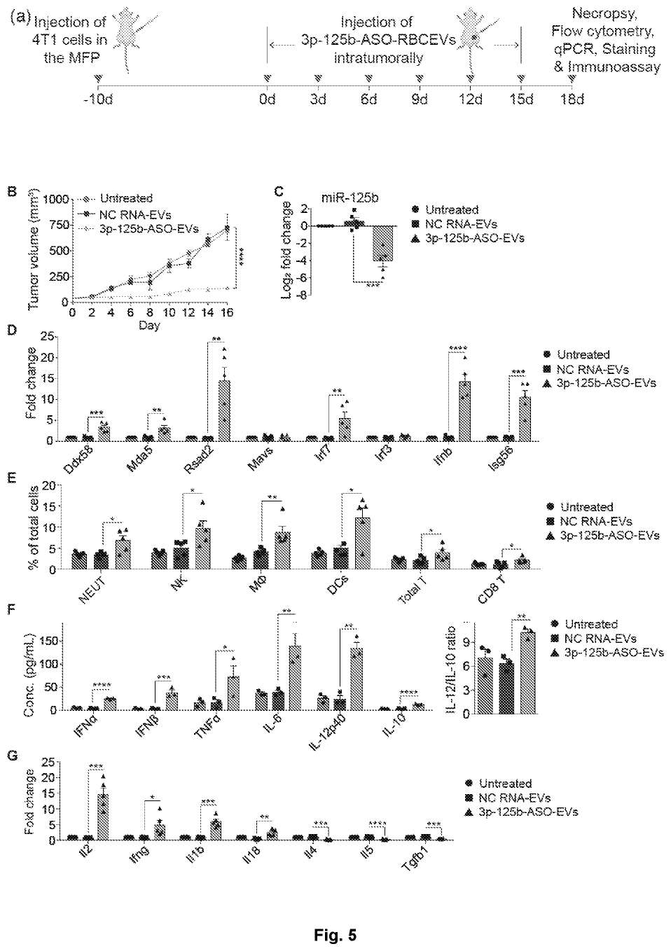

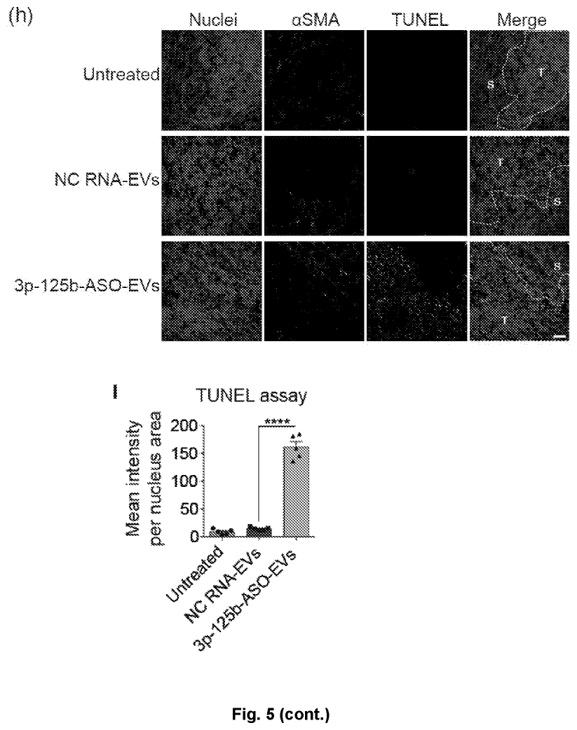

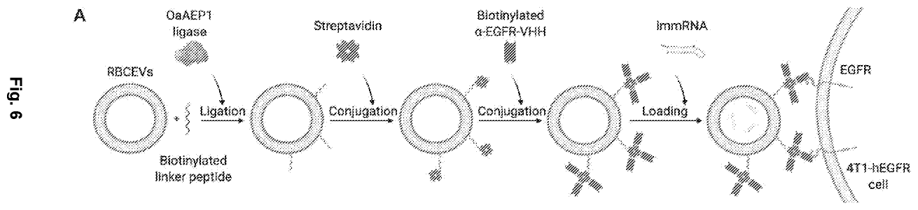

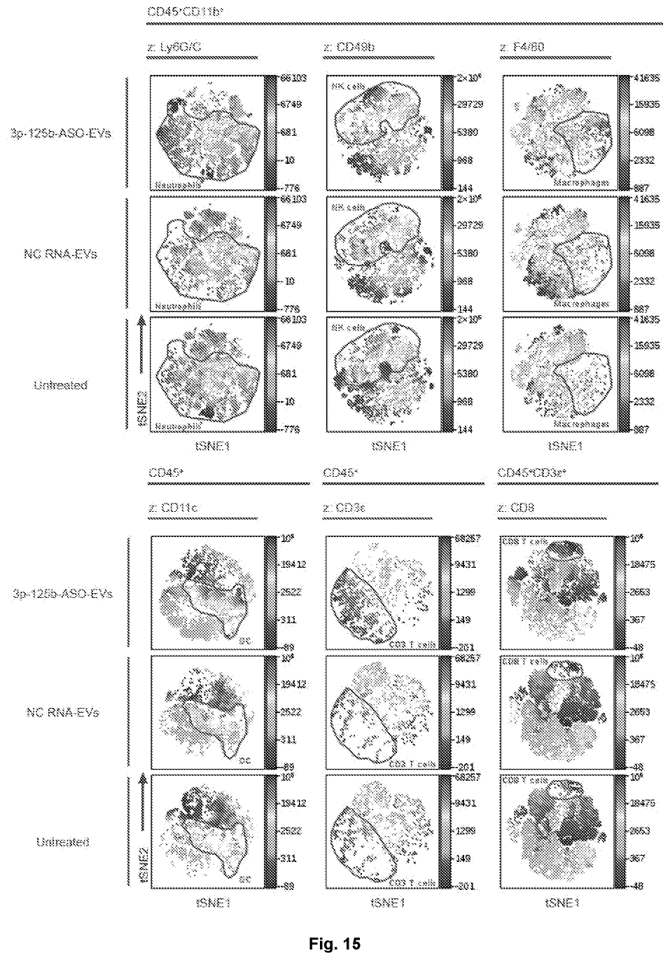

Next, the anti-tumour efficacy of 3p-125b-ASO was tested in 4T1 cancer model in vivo. 4T1 cells were implanted in the 4th MFP of BALB/c mice (FIG. 5A). 4T1 tumours were treated with 5 mg/kg 3p-125b-ASO-loaded RBCEVs every three days and monitored every two days (FIG. 5A). The mice were sacrificed and tumours were collected when the untreated tumours reached ˜15 mm in diameter. Similar to the immRNA treatment, it was observed that 3p-125b-ASO delivered using RBCEVs suppressed 4T1 tumour growth significantly (FIG. 5B). qPCR analysis of the dissociated tumour cells clearly showed decreased level of miR-125b and increased levels of Ddx58, Mda5, Mavs, Irf3, Irf7, Ifnb, Rsad2 and Isg56 in the tumours treated with 3p-125b-ASO-loaded RBCEVs as compared to NC-RNA-loaded RBCEVs (FIGS. 5C-D). Furthermore, FACS analysis revealed increased numbers of tumour-infiltrating neutrophils, NK cells, macrophages, dendritic cells, total T cells, and CD8+ T cells in the tumours treated with 3p-125b-ASO-loaded RBCEVs as compared with NC-RNA-loaded RBCEVs (FIGS. 5E and 15). As systemic low IFNβ level was detected by ELISA in the tumour-bearing mice receiving intratumoral administration of immRNA-RBCEVs, cytokine immunoassay was performed in the tumour lysates to improve the sensitivity in detecting the lowest concentration of cytokines. The data revealed that elevated levels of IFNα (˜25.62 pg/mL), IFNβ (˜38.71 pg/mL), TNFα (˜72.64 pg/mL), IL-6 (˜139.26 pg/mL), IL-12p40 (˜100.78 pg/mL) and IL-10 (˜23.06 pg/mL) were detected in the tumours treated with 3p-125b-ASO-loaded RBCEVs as compared to the controls (FIG. 5F). The IL-12/IL-10 ratio significantly increased upon 3p-125b-ASO-loaded RBCEV treatment (FIG. 5F), suggesting a proinflammatory tumour microenvironment. An additional qPCR analysis of the tumour cells showed significant increases in Il2, Ifng, Il1b, and Il18, and decreases in Il4, Il5, and Tgfb1 (FIG. 5G), indicative of Th1-dominant immune response. Indeed, 3p-125b-ASO-loaded RBCEVs substantially induced apoptosis with TUNEL positive cells concentrated in the tumour bulk but not in the stroma as determined by TUNEL staining of the tumour sections, suggesting that miR-125b knockdown and RIG-I activation contribute to the tumoricidal activity mediated by 3p-125b-ASO (FIGS. 5H-I). Together, these data provide evidence that both miR-125b inhibition and RIG-I-mediated activation enhanced anti-tumour efficacy of 3p-125b-ASO in an orthotopic breast cancer model.

Conjugation of RBCEVs with EGFR-Binding Nanobody Promotes Specific Delivery of immRNA to Metastatic EGFR-Positive Breast Cancer Cells

Targeting cancer cells specifically is a vital characteristic of EV-based drug delivery, as it enhances the therapeutic efficacy while protecting normal cells from toxicity. EGFR, known as a proto-oncogene in many types of cancers, is considered a common cancer biomarker. Mouse breast cancer 4T1 cells were transduced with lentiviruses carrying the human EGFR expression vectors. An RBCEV surface functionalization method was previously developed using OaAEP1 ligase to ligate peptides with ligase-binding motif (NGL) at the C-terminus onto RBCEV surface (Pham et al., 2021). In the present study, the enzymatic ligation method was combined with a streptavidinbiotin conjugation method to conjugate RBCEVs with nanobodies via a linker peptide (FIG. 6A). Specifically, RBCEVs were enzymatically ligated with a biotinylated linker peptide (biotin-TRNGL), which was sequentially conjugated with tetrameric streptavidin and biotinylated anti-EGFR nanobody (α-EGFR-VHH) (FIG. 6A). Flow cytometry analysis showed the specific binding of α-EGFR-VHH to human EGFR-expressing cells (CA1a, H358, and 4T1-hEGFR cells) rather than mouse EGFR-expressing 4T1 cells (FIG. 11C). To assess the specificity of EGFR-targeting RBCEVs towards EGFR-expressing cancer cells, EGFR-VHH-coated RBCEVs were labelled with CFSE and incubated with 4T1 cells expressing human EGFR (4T1-hEGFR) and parental 4T1 cells at a suboptimal dose for 2 hours. An anti-mCherry nanobody was used as a negative control (Ctrl-VHH). The CFSE fluorescence intensity in 4T1-hEGFR cells treated with EGFR-targeting CFSE-labelled RBCEVs was ˜28.6-fold higher than that in 4T1-hEGFR cells treated with non-targeted CFSE-labelled RBCEVs (FIG. 6B). The non-targeted CFSE-labelled RBCEVs were taken up by 4T1-hEGFR cells at similar levels as non-targeted and EGFR-targeted CFSE-labelled RBCEVs by 4T1 cells (FIG. 6B). The specific uptake of EGFR-targeting RBCEVs carrying fluorescent RNAs was observed in 4T1-hEGFR cells. Consistently, the FAM fluorescence intensity was significantly higher in hEGFR-positive 4T1 cells treated with EGFR-VHH-coated FAM-ASO-loaded RBCEVs compared to parental 4T1 cells treated with EGFR-VHH-coated FAM-ASO-loaded RBCEVs and 4T1-hEGFR cells treated with non-targeted FAM-ASO-loaded RBCEVs (FIG. 6C). To verify if the EGFR-targeting RBCEVs specifically deliver functional RNAs to 4T1-hEGFR cells, EGFR-VHH-coated RBCEVs were loaded with immRNA. The EGFR-VHH-coated immRNA-loaded RBCEVs exhibited a higher functional uptake by 4T1-hEGFR cells compared to 4T1 cells as evidenced by the substantial increases in cellular Ddx58, Rsad2 and Ifnb expression (FIGS. 6D and 16A).

To generate a lung metastatic breast cancer model, 4T1-hEGFR cells were injected intravenously in the tail vein of BALB/c mice (FIG. 6E). After seven days, non-targeted and EGFR-targeted CFSE-labelled RBCEVs were delivered via intrapulmonary administration to the mice bearing metastatic breast cancer. CFSE-labelled RBCEVs were washed extensively using size exclusion chromatography and centrifugation. The flowthrough of the last wash was used as a negative control. 24 hours after intrapulmonary delivery, the biodistribution of non-targeted and EGFR-targeted CFSE-labelled RBCEVs in the lungs were analyzed using flow cytometry. The intrapulmonary delivery of EGFR-targeted and non-targeted RBCEVs was consistent as shown by similar percentage (˜16%) of CFSE-positive cells in the homogenized whole lung cells (FIGS. 6F and 16B). The conjugation of RBCEVs with EGFR nanobody increased the percentage of CFSE-positive tumour cells to ˜68% (FIGS. 6F and 16B). No significant change was observed in alveolar macrophages, which are located in the airway lumen and always took EVs up at a high rate (FIGS. 6F and 16B). In addition, a significant decrease in the percentage of CFSE-positive neutrophils and a slight decrease in the percentage of CFSE-positive interstitial macrophages were observed in the lungs of mice treated with EGFR-targeting RBCEVs (FIGS. 6F and 16B). This can be attributed to the hEGFR-specific RBCEVs being preferentially taken up by hEGFR-positive tumour cells over the hEGFR-negative cells in the lung parenchyma. These data suggest that EGFR nanobody conjugation can drive RBCEVs specifically towards EGFR-positive metastatic breast cancer cells in vivo.

Intrapulmonary Delivery of immRNA Using EGFR-Targeted RBCEVs Suppresses Breast Cancer Metastasis in the Lung

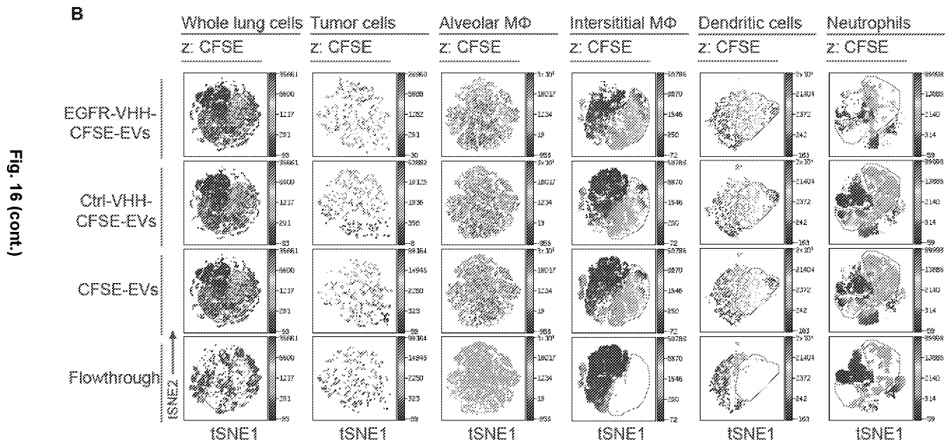

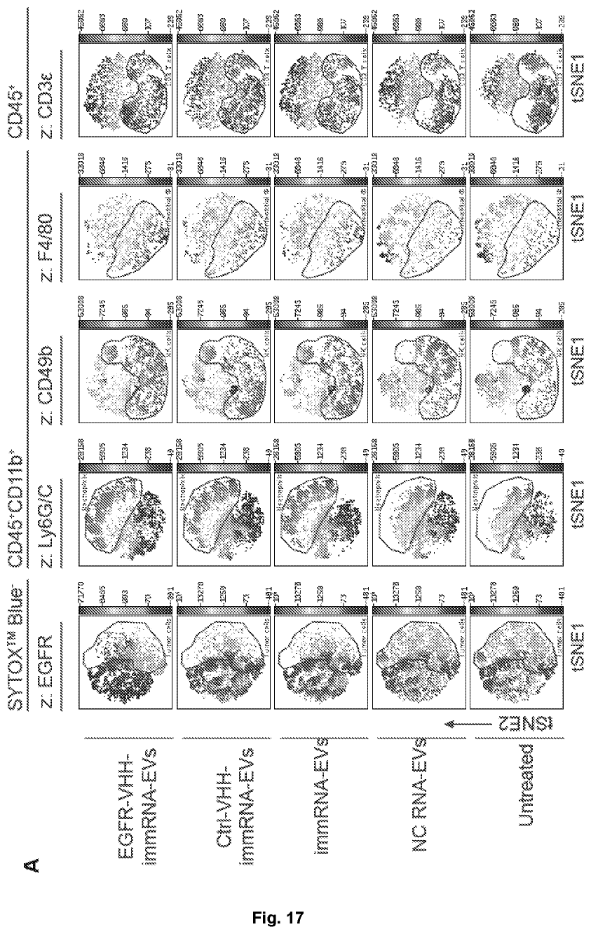

In order to treat metastatic breast cancer in the lung, multiple doses of EGFR-targeting immRNA-loaded RBCEVs were delivered via intrapulmonary administration every other day (FIG. 7A). After 5 treatments, the mice were sacrificed and the lungs were excised for subsequent analysis. As expected, non-targeted and EGFR-targeted immRNA-loaded RBCEVs suppressed the engraftment of metastatic tumour cells as shown by the decreased percentage of hEGFR-positive cells in the lungs (FIGS. 7B, C, and 17A). In particular, EGFR-targeted immRNA-loaded RBCEVs repressed tumour growth in the lungs, which is an effect attributed to the increased apoptosis in this treatment. H & E staining of the lung sections showed that the metastatic areas of lungs were significantly lower in the mice treated with EGFR-targeted immRNA-loaded RBCEVs (FIGS. 7D-E). qPCR analysis of the whole lung lysate revealed that immRNA-loaded RBCEVs triggered RIG-I cascade activation by up-regulating Ddx58, Mda5, Rsad2, Irf7, Ifnb and Isg56. Although there was no substantial increase in RLR genes, Ifnb and IFN-stimulated genes Isg56 and Rsad2 increased significantly upon EGFRtargeted RBCEVs treatment compared to non-targeted RBCEVs treatment (FIG. 7F). According to FACS analysis, increased infiltrations of neutrophils, NK cells, interstitial macrophages, cDCs, T cells including CD8+ T cells but not CD4+ T cells were observed in the lungs of mice treated with immRNA-loaded RBCEVs (FIGS. 7G and 17A). EGFR-targeted RBCEVs further increased the number of infiltrating neutrophils, interstitial macrophages, cDCs, T cells and CD8+ T cells (FIGS. 7G and 17A). Alveolar macrophages were almost eliminated in the airways of mice treated with the control and EGFR-targeted RBCEVs containing immRNA (FIGS. 7G and 17A). TUNEL staining of the lung sections further confirmed that the treatment with EGFR-targeted RBCEVs containing immRNA increased apoptosis in the lungs compared to the treatment with non-targeted immRNA-loaded RBCEVs (FIGS. 7H-I). To investigate whether the respiratory inflammatory state in mice affects metastasis, the concentrations of cytokines were detected in the lung homogenates. All tested cytokines were significantly elevated in the lungs of mice treated with EGFR-targeted and nontargeted immRNA-loaded RBCEVs versus the control mice (FIG. 7J). The mice receiving EGFR-targeted immRNA-loaded RBCEVs exhibited even higher IFNα (˜17.97 pg/mL), IFNβ (˜22.47 pg/mL), TNFα (˜312.86 pg/mL), IL-12p40 (˜72.73 pg/mL), IL-10 (˜16.91 pg/mL), and IL-12/IL-10 ratio, as compared with those receiving non-targeted immRNA-loaded RBCEVs, whereas there was no significant difference in the levels of IL-6 (FIG. 7J). qPCR analysis of the lung lysate revealed that immRNA-loaded RBCEVs increased the mRNA levels of Il2, Ifng, Il1b, and Il18, while decreasing the levels of Il4, Il5, and Tgfb1 (FIG. 17B), indicating Th1-dominant immunity in the lung. EGFR-targeted RBCEVs further enhanced the expression of Il2 and Ifng, and reduced the expression of Tgfb1, as compared to non-targeted RBCEVs (FIG. 17B). Taken together, these data suggest that EGFR-targeted RBCEVs enhance the specificity of delivery to EGFR-positive cancer cells leading to improved therapeutic efficacy of immRNA in vivo.

EGFR-Targeted immRNA-Loaded RBCEVs Induce DC Activation, Promote M1 Macrophage Polarization and Potentiate Tumour-Specific CD8+ T Cell Activity

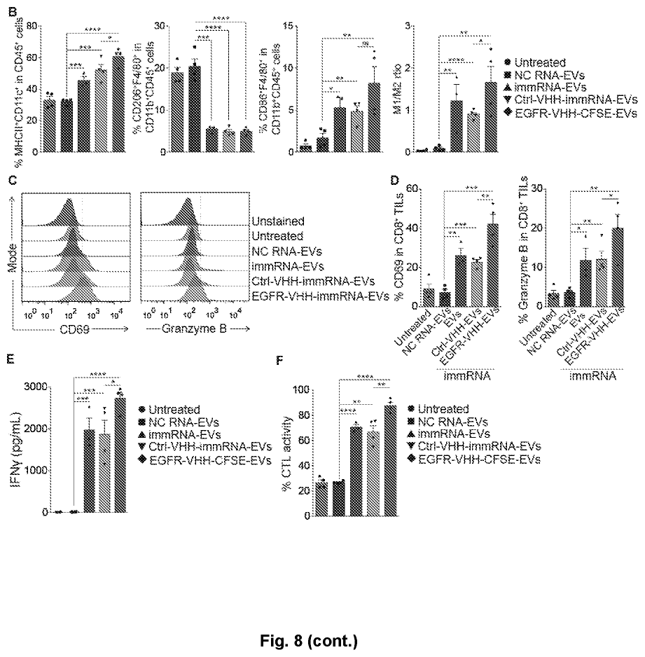

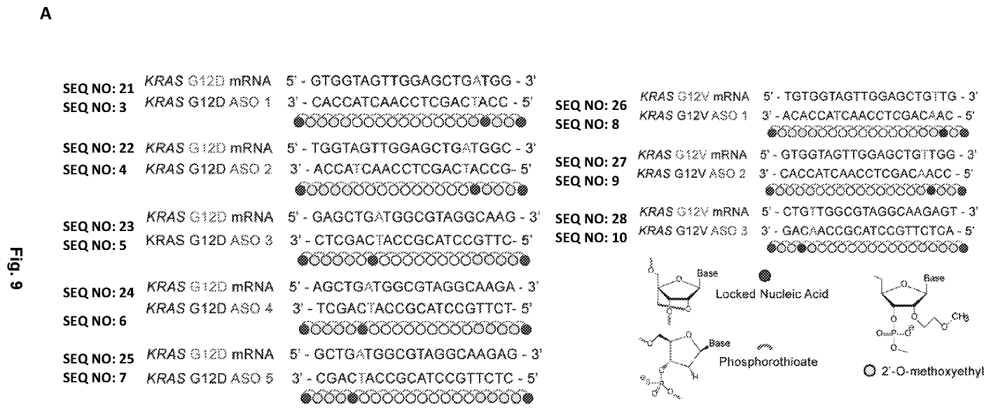

To assess the ability of EGFR-targeted immRNA-loaded RBCEVs to induce DC activation and regulate macrophage polarization in the lung, the expression of DC activation marker and macrophage M1/M2 markers were determined using flow cytometry. The data revealed that non-targeted and EGFR-targeted immRNA-loaded RBCEVs treatments elevated the percentage of MHCII+CD11c+ DCs in CD45+ cells (FIGS. 8A-B). EGFR-targeted immRNA-loaded RBCEVs treatment further increased the percentage of MHCII+CD11c+ DCs as compared to non-targeted immRNA-loaded RBCEVs treatment (FIGS. 8A-B). In addition, immRNA-loaded RBCEVs treatment led to lower percentage of CD206+F4/80+M2-like macrophages and higher percentage of CD86+F4/80+M1-like macrophages in CD11b+CD45+ cells (FIGS. 8A-B). The M1/M2 ratio significantly increased upon EGFR-targeted immRNA-loaded RBCEVs treatment as compared to non-targeted immRNA-loaded RBCEVs treatment (FIG. 8B). To quantify the activity of CD8+ tumour-infiltrating lymphocytes (TILs) against tumour-associated antigens, CD8+ TILs were isolated from the lungs for cytotoxicity analysis. As a result, CD69 and granzyme B levels significantly increased in CD8+ TILs upon immRNA-loaded RBCEV treatments (FIGS. 8C-D). Given that the tumour-specific immune response is a key factor in cancer immunotherapy, the tumour-specific T cell response was evaluated. CD8+ TILs were incubated with 4T1-hEGFR-luciferase cells at a ratio of 50:1 for 48 h. Re-stimulation of isolated CD8+ TILs from immRNA-loaded RBCEVs treated mice with tumour antigens markedly augmented the production of IFNγ, which is associated with a significant increase in cytotoxic T lymphocytes (CTL) activity (FIGS. 8E-F). EGFR-targeted immRNA-loaded RBCEVs further potentiated the cytotoxicity of CD8+ TILs as demonstrated by the increased cellular expression of CD69 and granzyme B, secretion of IFNγ and CTL activity (FIGS. 8C-F). Collectively, these data correlated with the elevated expression of Th1 cytokines and reduced expression of Th2 cytokines in the lungs of EGFR-targeted immRNA-RBCEVs treated mice (FIGS. 7J and 17B), indicating Th1-associated and CTL-mediated anti-tumour immune responses.

### Specific Knockdown of Mutant KRAS and EGFR in Various Types of Cancers Using RBCEVs-Delivered ASOs

Cancer cells elicit numerous genetic alterations to support the tumor progression by providing survival and proliferation advantages as well as by forming poorly immunogenic tumor microenvironments (TMEs). The most common oncogenic driver in cancer is KRAS activating mutation, found in ˜30% of patients with lung cancer and colorectal carcinoma, and nearly 100% patients with pancreatic cancer. Most KRAS-mutant cancer cells carry a mismatch mutation at glycine 12 (G12). Although ARS1620, a small molecule drug, is recently invented as an effective inhibitor of KRAS-G12C, it is not applicable to other KRAS mutants, even other mismatches of G12. RBCEVs were used herein to deliver ASO drugs targeting oncogenic drivers, including KRAS G12D, KRAS G12V, EGFR L858R, and EGFR T790M, where the combination of oncogene-targeting ASO and immRNA achieved a superior anti-cancer effect.

The ASOs were designed using a Gapmer approach. The ASOs were chemically modified with locked nucleic acid (LNA) at 2 ends and the nucleotide matching with the missense mutation to increase specificity. In addition, phosphorothioate (PS) modification was included in every nucleotide and the 2′-O-methoxyethyl (2′MOE) modification was added to the five nucleotides at each wing to increase stability of the ASO (FIGS. 9A and 18A). KRAS mutant-bearing cancer cell lines, A427 (KRAS G12D), AsPC-1 (KRAS G12D) and H441 (KRAS G12V) were used to examine the specific knockdown by the corresponding ASO-loaded RBCEVs. H1975 and DU145 cells, which harbor WT KRAS gene, were used as control cell lines. By Western Blot and qPCR analysis, it was found that KRAS G12D ASO 2 and KRAS G12V ASO 2 showed significant knockdown efficacy of the corresponding mutant gene in A427, AsPC-1, and H441 cells, respectively, resulting in noticeably reduced viability of mutant-bearing cells determined by CCK-8 assay, while minimizing the cross reaction in WT gene-harboring H1975 and DU145 cells (FIG. 9B-I).

Similarly, the specific knockdown of EGFR L858R and T790M mutations by ASOs delivered by RBCEVs in H1975 cells bearing these two mutations was examined. A549 cells which carry WT EGFR were used as a control cell line (FIG. 18A). FIG. 18B-G revealed that EGFR L858R ASO4 and T790M ASO4 specifically inhibited EGFR protein expression in target cells while showing limited effect on the EGFR WT in A549 control cells. Based on these data, KRAS G12D ASO 2, KRAS G12V ASO 2, EGFR L858R ASO 4, and EGFR T790M ASO 4 was selected for further examination.

Combination of Oncogene Addiction-Targeting ASO with immRNA Synergistically Potentiates the RIG-I Pathway Activation in Cancer Cells

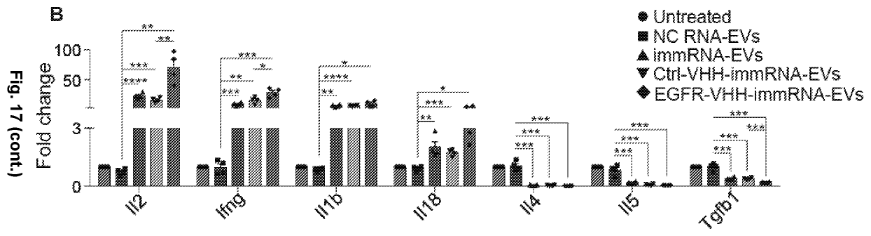

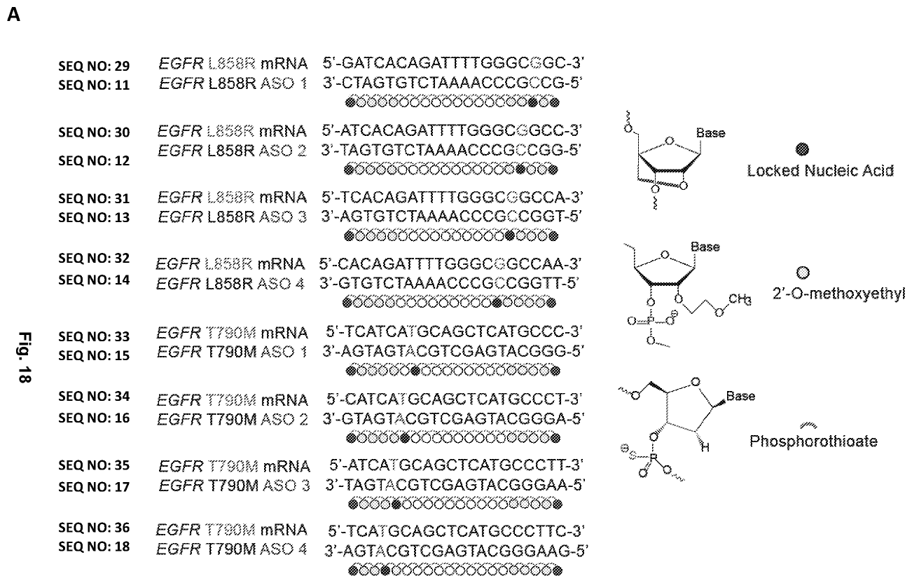

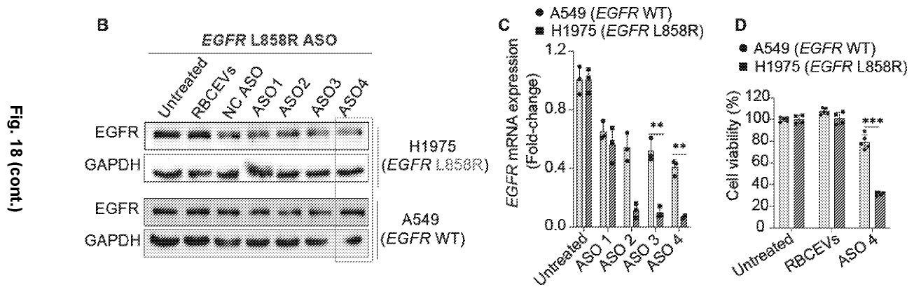

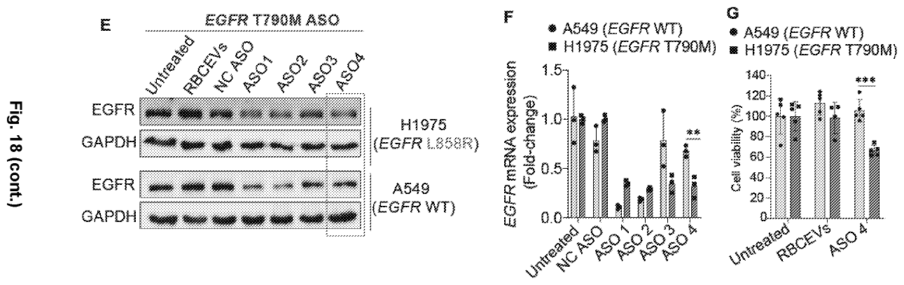

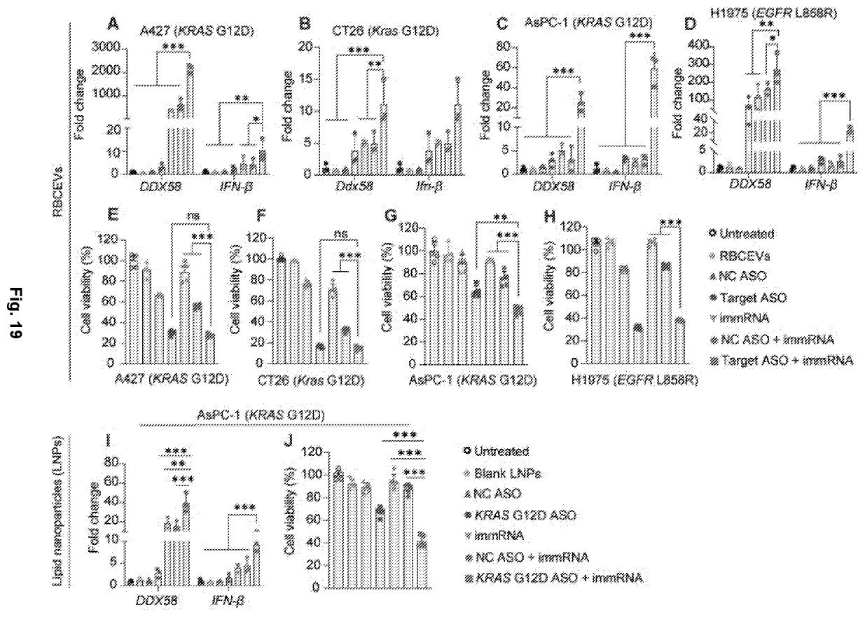

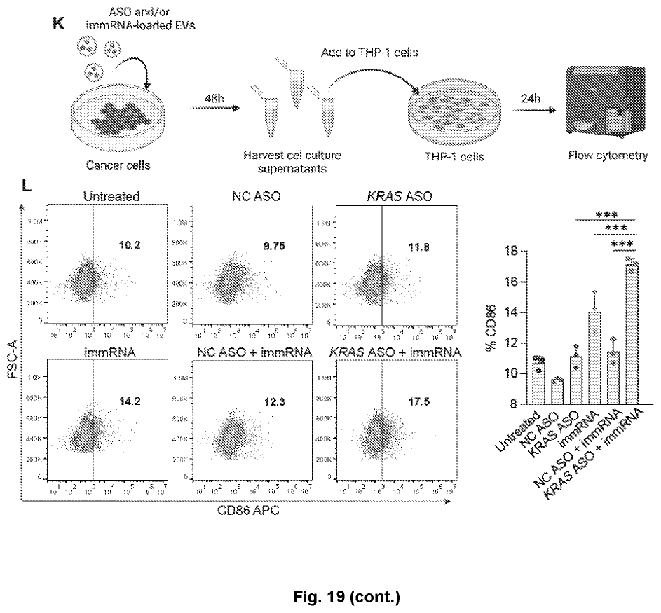

The anti-cancer potency of the combinatorial regime of oncogene addiction inhibition and RIG-I activation by co-delivering the ASO and immRNA to various mutant-bearing cancer cell lines, including A427 (KRAS G12D) lung cancer cells, CT26 (Kras G12D) colorectal cancer cells, AsPC-1 (KRAS G12D) pancreatic cancer cells, H441 (KRAS G12V) lung cancer cells, and H1975 (EGFR L858R/T790M) lung cancer cells, using the RBCEV and LNP platforms, was investigated. The expression level of DDX58 and IFN-β in treated cells was first quantified using qPCR. Notably, the cells treated with immRNA in combination with either KRAS ASO or EGFR ASO exhibited highest expression of both DDX58 and IFN-β than those received single treatment (FIG. 19A-D). Next, cancer cell viability was determined after various treatments with RBCEV formulations by CCK-8 assay (FIGS. 19E-H) and it was found that this combination exerted significantly enhanced cytotoxicity against KRAS G12D-expressing pancreatic AsPC-1 cells (FIG. 19G). A similar observation was found when the AsPC-1 cells were treated with ASO and immRNA delivered by LNPs (FIG. 19I-J).

The activation of THP-1 monocytic cells in a co-culture with pre-treated AsPC-1 cells was next examined (FIG. 19K). By flow cytometric analysis, it was found that AsPC-1 cells pretreated with KRAS ASO and immRNA expressed an elevated level of CD86, an activation marker of monocytic THP-1 cells (FIG. 19L).

RBCEVs Delivered with EGFR ASO Effectively Inhibit Lung Tumor Growth

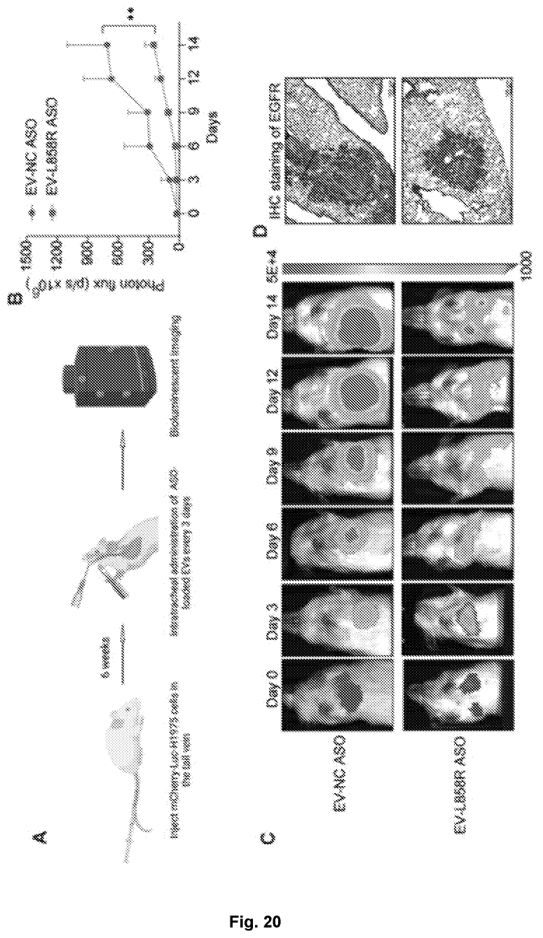

To investigate the therapeutic potential of mutant-specific ASOs in vivo, H1975 tumor cell xenografted mice was generated via intravenous injection. EGFR L858R ASO was loaded onto EVs and delivered into tumor-bearing mice intratracheally every 3 days (FIG. 20A). Noticeably, EGFR L858R ASO-loaded EVs displayed a remarkable tumor inhibition effect, indicated via a significantly lower luciferase signal than that of mice received NC ASO-loaded EVs (FIG. 20B-C). By immunohistochemistry (IHC) staining, a noticeable decrease in EGFR level in the L858R ASO-EV-treated group, compared to that in mice treated with NC ASO-loaded EVs (EV-NC ASO), was observed (FIG. 20D).

RBCEV-Mediated Combination of immRNA with Oncogene-Targeting ASOs Exhibit a Potent Anti-Cancer Effect In Vivo

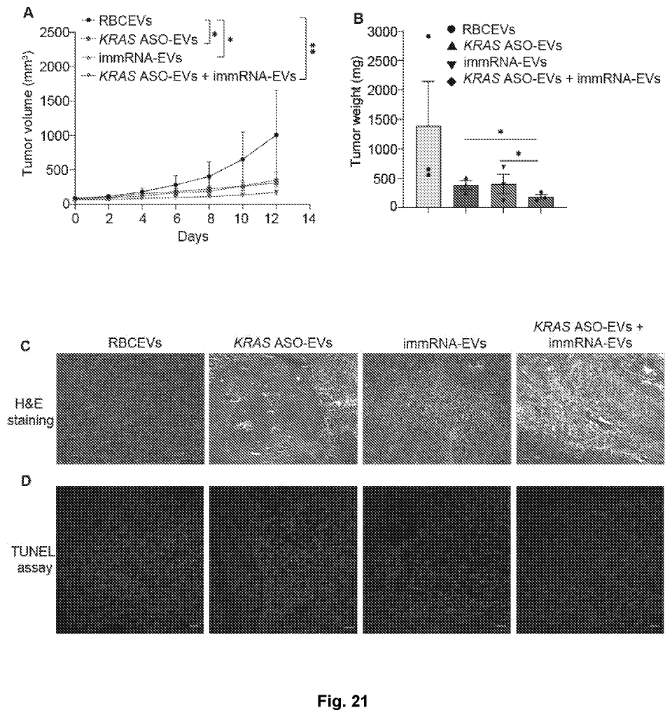

A mouse model of syngeneic CT26 colorectal cancer with KRAS G12D mutation was generated by subcutaneously (s.c) inoculating CT26 colorectal cancer cells into BALB/c mice. RBCEVs loaded with KRAS ASO, immRNA, or combined KRAS ASO and immRNA were injected into the tumors every two days. It was observed that the combination of KRAS G12D ASO and immRNA exhibited a superior tumor inhibition effect, indicated by the reduced tumor volume (FIG. 21A) and tumor weight (FIG. 21B). Moreover, by H&E staining (FIG. 21C) and TUNEL assay (FIG. 21D), it was also observed that there was a decrease in tumor cell density and formation of necrotic areas in tumors collected from mice received the combination treatment. These results suggest the potential of a combination regimen of KRAS inhibiting ASO and RIG-I activation using RBCEV platform for KRAS driven cancer.

In summary, a robust platform for local and targeted delivery of immunomodulatory RIG-I agonists using RBCEVs was demonstrated. The treatments of orthotopic and metastatic breast cancers with both immRNA- and 3p-125b-ASO-loaded RBCEVs allowed activation of RIG-I signalling, which results in a three-pronged attack: (i) strong tumour growth inhibition; (ii) augmented levels of type I IFN and pro-inflammatory cytokines; and (iii) IFN-mediated recruitment of innate immune cells (macrophages, neutrophils, NK cells), activation of dendritic cells and M1 macrophage polarization, and cross-priming of adaptive immune effectors (CD8+ T cells) through antigen-presenting cells (dendritic cells and macrophages) (FIG. 10), indicating a reversal of the immunosuppressive tumour microenvironment. Polarization of Th1/Th2 immune factors promotes anti-tumour immunocompetence. Th1 cytokines, such as IL-1B, IL-2, IL-12, IL-18, TNFα, and IFNγ, are associated with proinflammation, while Th2 cytokines, such as IL-4, IL-5, and TGFβ, play a suppressive role in the tumour immune microenvironment. The data demonstrated that immRNA- and 3p-125b-ASO-loaded RBCEVs altered the Th1/Th2 balance toward Th1 dominance in the tumour microenvironment contributing to tumour suppression. Effective cancer immunotherapy requires T cell priming, activation and tumour infiltration. Hence, intratumoral delivery of immRNA- and 3p-125b-ASO-loaded RBCEVs induced massive cell apoptosis in the tumour bulk, whereas tumour stroma showed no signs of apoptosis.

I ImmRNA-loaded RBCEVs and anti-PD-L1 were combined for the enhanced anti-tumour activity. However, ˜50% mortality was observed in tumour-bearing mice receiving repeated doses of systemic anti-PD-L1 administration. The fatality of mice might be attributed to the hypersensitivity reactions caused by anti-PD-L1. These findings highlight the previously uncharacterized adverse toxicity of anti-PD-L1 not reported in previous studies of RIG-I-agonist and anti-PD-L1 combination. Indeed, various clinical studies have demonstrated that anti-PDL1 as a single treatment greatly induced systemic IFNβ release and cytokine storm i. Systemic administration of RIG-I agonists is likely to trigger the cytokine release syndrome as well but local administration of immRNA is safe and sufficiently effective according to the data disclosed herewith.

A combinatorial approach was demonstrated to simultaneously inhibit oncogenic miR-125b and activate the RIG-I pathway. Due to their genetic and epigenetic plasticity, tumour cells tend to evade single-targeted therapies such as specific kinase/oncogene inhibitors or immunotherapies. Therefore, multi-targeted therapies are needed. 3p-125b-ASO offers advantages over combinations of multiple single-targeted therapies. It's small and easy to synthesize. It comprises two distinct and independent properties of RIG-I activator and oncogene suppressor. Moreover, RIG-I induced apoptosis of cancer cells synergized with apoptosis induced by ASO-mediated inhibition of miR-125b. Such bi-functional ASOs can be adapted to different tumour entities by targeting key oncogenes that drive tumorigenicity. Moreover, the synergistic anti-cancer effect was observed when targeting both KRAS-G12D mutant and the RIG-I pathway in pancreatic cancer, at least at in vitro setting. These results suggest the potential of immRNA for inflaming the poorly immunogenic “cold” status of pancreatic tumour, which is greatly contributed by KRAS mutations. Collectively, these data suggest the potential of immRNA, 3p-125b-ASO and KRAS-G12D ASO for clinical application.

Biosafety and reproducibility of drug delivery systems are critical for their clinical translation. In vivo-jetPEI is a hitherto widely used delivery system for RIG-I agonists and other kinds of RNA therapeutics in preclinical and clinical studies. However, it has been reported to be associated with fatal hepatotoxicity in mice. Owing to their biocompatibility, stability and limited immunogenicity, EVs provide multiple advantages as a delivery system over traditional synthetic delivery vehicles. RBCEVs with high availability and scalability and without risk of horizontal gene transfer have been illustrated to be biosafe, efficient and amendable for therapeutic delivery in cancer treatment Effective immunomodulation of the tumour microenvironment and potent anti-tumor activity using RBCEVs as delivery vehicles for RIG-I agonists without any observable adverse effects was achieved, suggesting the advantages of RBCEVs for clinical application.

An RBCEV surface functionalization method with EGFR-targeted nanobodies was also described, which can enhance the delivery of RIG-I agonists toward EGFR-positive cancer cells, thereby improving therapeutic efficacy while reducing side effects. In the experiments, a depletion of alveolar macrophages in the respiratory tract was observed upon intrapulmonary delivery of non-targeted and EGFR-targeted RBCEVs containing immRNA. From the data on biodistribution of intrapulmonary delivered RBCEVs, alveolar macrophages were the primary recipient of both non-targeted and EGFR-targeted RBCEVs. It is evident that RBCEVs themselves have no influence on alveolar macrophages because CFSE-labeled or NC-RNA-loaded RBCEVs did not cause rapid death of these cells. The alveolar macrophages are assigned an important role in removing antigens in the lungs by nonspecific phagocytosis. The depletion of alveolar macrophages might be an effect attributed to RIG-I-mediated immune responses from phagocytized RBCEVs containing immRNA. Thus, described herein is the EV-mediated delivery of therapeutic RNA via intrapulmonary administration. The data show that RNA-loaded RBCEVs are stable and able to penetrate deeply into the lung parenchyma for the delivery of immRNA. The composition described herein can be used for therapeutic RNA delivery that is applicable not only to cancer metastasis but also to other pulmonary diseases such as influenza and asthma.

TABLE 1
List of primers

| | Forward (F)/ | | SEQ | | SEQ |
|---|---|---|---|---|---|
| | Reverse (R) | | ID | | ID |
| Genes | strand | Mouse | NO: | Human | NO: |
| GAPDH | F | AGGTCGGTGTGAACGGATTTGT | 59 | GGAGCGAGATCCCTCCAAAAT | 61 |
| | R | GTAGACCATGTAGTTGAGGTCA | 60 | GGCTGTTGTCATACTTCTCATG | 62 |
| | | | | G | |
| DDX58 | F | GAG AGT CAC GGG ACC | 63 | GCCATTACACTGTGCTTGGAGA | 65 |
| | | CAC T | | | |
| | R | CGG TCT TAG CAT CTC | 64 | CCAGTTGCAATATCCTCCACCA | 66 |
| | | CAA CG | | | |
| MDA5 | F | TGATGCACTATTCCAAGAACTA | 67 | GAGCAACTTCTTTCAACCACAG | 69 |
| | | AC | | | |
| | R | TCTGTGAGACGAGTTAGCCAAG | 68 | CACTTCCTTCTGCCAAACTTG | 70 |
| RSAD2 | F | ACAGCCAAGACATCCTTCGT | 71 | CACAAAGAAGTGTCCTGCTTGG | 73 |
| | | | | T | |
| | R | AAAAGTTGATCTTCTCCAAACC | 72 | AAGCGCATATATTCATCCAGAA | 74 |
| | | A | | T | |
| MAVS | F | CTGCCTCACAGCTAGTGACC | 75 | AGGAGACAGATGGAGACACA | 77 |
| | R | CCGGCGCTGGAGATTATTG | 76 | CAGAACTGGGCAGTACCC | 78 |
| IFNB | F | AACCTCACCTACAGGGCGGACT | 79 | CTCTCCTGTTGTGCTTCTCC | 81 |
| | | TC | | | |
| | R | TCCCACGTCAATCTTTCCTCTT | 80 | GTCAAAGTTCATCCTGTCCTTG | 82 |
| | | GCT | | | |
| IRF3 | F | CGGAAAGAAGTGTTGCGGTT | 83 | ACCAGCCGTGGACCAAGAG | 85 |
| | R | TTTTCCTGGGAGTGAGGCAG | 84 | TACCAAGGCCCTGAGGCAC | 86 |
| IRF7 | F | AGGGCGTTTTATCTTGCG | 87 | TGGTCCTGGTGAAGCTGGAA | 89 |
| | R | TGGAGCCCAGCATTTTCTCT | 88 | GATGTCGTCATAGAGGCTGTTG | 90 |
| | | | | G | |
| EGFR | F | | | GTGTGCCACCTGTGCCATCC | 91 |
| | R | | | GCCACCACCAGCAGCAAGAG | 92 |
| ISG56 | F | TACAGGCTGGAGTGTGCTGAGA | 93 | TAGCCAACATGTCCTCACAGAC | 95 |
| | R | CTCCACTTTCAGAGCCTTCGCA | 94 | TCTTCTACCACTGGTTTCATGC | 96 |
| IL4 | F | ATCATCGGCATTTTGAACGAGG | 97 | | |
| | R | GCAGCTCCATGAGAACACTA | 98 | | |
| IL5 | F | CTCTGTTGACAAGCAATGAGAC | 99 | | |
| | | G | | | |
| | R | TCTTCAGTATGTCTAGCCCCTG | 100 | | |
| IFNG | F | GCCACGGCACAGTCATTGA | 101 | | |
| | R | TGCTGATGGCCTGATTGTCTT | 102 | | |
| IL2 | F | GTGCTCCTTGTCAACAGCG | 103 | | |
| | R | GGGGAGTTTCAGGTTCCTGTA | 104 | | |
| TGFB1 | F | CTTCAATACGTCAGACATTCGG | 105 | | |
| | | G | | | |
| | R | GTAACGCCAGGAATTGTTGCT | 106 | | |
| | | A | | | |
| ILIB | F | GCAACTGTTCCTGAACTCAACT | 107 | | |
| | R | ATCTTTTGGGGTCCGTCAACT | 108 | | |
| IL18 | F | GACTCTTGCGTCAACTTCAAGG | 109 | | |
| | R | CAGGCTGTCTTTTGTCAACGA | 110 | | |
| IL10 | F | AGCCTTATCGGAAATGATCCAG | 111 | | |
| | | T | | | |
| | R | GGCCTTGTAGACACCTTGGT | 112 | | |
| IL6 | F | TCTATACCACTTCACAAGTCGG | 113 | | |
| | | A | | | |
| | R | TCTATACCACTTCACAAGTCGG | 114 | | |
| | | A | | | |
| IL12B | F | TGGTTTGCCATCGTTTTGCTG | 115 | | |
| | R | ACAGGTGAGGTTCACTGTTTCT | 116 | | |
| TNF | F | CCTGTAGCCCACGTCGTAG | 117 | | |
| | R | GGGAGTAGACAAGGTACAACCC | 118 | | |
| IFNA4 | F | CTGGTAATGATGAGCTACTACT | 119 | | |
| | | GG | | | |
| | R | CCTTCTCCAAGGGGAATCCAA | 120 | | |
| IFNAII | F | GGTCCTGGCACAAATGAGGA | 121 | | |
| | R | TCCAAGCAGCAGATGAGTCC | 122 | | |
| IFNA12 | F | AAGACTGAGTGAGAAGGAGTGA | 123 | | |
| | | G | | | |
| | R | GAGATGCCAGAATTTGAGCAGT | 124 | | |
| | | G | | | |
| immRNAs | F | | | GGATTTCCCCCGAAGGTGGAAA | 125 |
| | | | | TCCTATAGTGAGTCGTATTAC | |
| | R | | | GTAATACGACTCACTATAGGAT | 126 |
| | | | | TTCCACCTTCGGGGGAAATCC | |
| 3p-125b- | F | | | AGCCTGAGACCCTAACTTCCTA | 127 |
| ASOs | | | | TAGTGAGTCGTATTAC | |
| | R | | | GTAATACGACTCACTATAGGAA | 128 |
| | | | | GTTAGGGTCTCAGGCT | |

TABLE 2
List of sequences and corresponding SEQ ID NO

| SEQ | | |
|---|---|---|
| ID | | |
| NO: | Name | Sequence (5′ to 3′) |
| 1 | Immunomodulatory RNA | GGAUUUCCACCUUCGGGGGAAAUCC |
| 2 | miRNA-125b-ASO | GGAAGUUAGGGUCUCAGGCCCUAACUUCC |
| 3 | KRAS G12D ASO 1 | CCATCAGCTCCAACTACCAC |
| 4 | KRAS G12D ASO 2 | GCCATCAGCTCCAACTACCA |
| 5 | KRAS G12D ASO 3 | CTTGCCTACGCCATCAGCTC |
| 6 | KRAS G12D ASO 4 | TCTTGCCTACGCCATCAGCT |
| 7 | KRAS G12D ASO 5 | CTCTTGCCTACGCCATCAGC |
| 8 | KRAS G12V ASO 1 | CAACAGCTCCAACTACCACA |
| 9 | KRAS G12V ASO 2 | CCAACAGCTCCAACTACCAC |
| 10 | KRAS G12V ASO 3 | ACTCTTGCCTACGCCAACAG |
| 11 | EGFR L858R ASO 1 | GCCGCCCAAAATCTGTGATC |
| 12 | EGFR L858R ASO 2 | GGCCGCCCAAAATCTGTGAT |
| 13 | EGFR L858R ASO 3 | TGGCCGCCCAAAATCTGTGA |
| 14 | EGFR L858R ASO 4 | TTGGCCGCCCAAAATCTGTG |
| 15 | EGFR T790M ASO 1 | GGGCATGAGCTGCATGATGA |
| 16 | EGFR T790M ASO 2 | AGGGCATGAGCTGCATGATG |
| 17 | EGFR T790M ASO 3 | AAGGGCATGAGCTGCATGAT |
| 18 | EGFR T790M ASO 4 | GAAGGGCATGAGCTGCATGA |
| 19 | modified immunomodulatory RNA (immRNA) sequence | pppGGAUUUCCACCUUCGGGGGAAAUCC |
| 20 | modified miRNA-125b-ASO sequence | pppGGAAGUUAGGGUCUCAGGCCCUAACUUCC |
| 21 | KRAS G12D ASO 1 target sequence | GTGGTAGTTGGAGCTGATGG |
| 22 | KRAS G12D ASO 2 target sequence | TGGTAGTTGGAGCTGATGGC |
| 23 | KRAS G12D ASO 3 target sequence | GAGCTGATGGCGTAGGCAAG |
| 24 | KRAS G12D ASO 4 target sequence | AGCTGATGGCGTAGGCAAGA |
| 25 | KRAS G12D ASO 5 target sequence | GCTGATGGCGTAGGCAAGAG |
| 26 | KRAS G12V ASO 1 target sequence | TGTGGTAGTTGGAGCTGTTG |
| 27 | KRAS G12V ASO 2 target sequence | GTGGTAGTTGGAGCTGTTGG |
| 28 | KRAS G12V ASO 3 target sequence | CTGTTGGCGTAGGCAAGAGT |
| 29 | EGFR L858R ASO 1 target sequence | GATCACAGATTTTGGGCGGC |
| 30 | EGFR L858R ASO 2 target sequence | ATCACAGATTTTGGGCGGCC |
| 31 | EGFR L858R ASO 3 target sequence | TCACAGATTTTGGGCGGCCA |
| 32 | EGFR L858R ASO 4 target sequence | CACAGATTTTGGGCGGCCAA |
| 33 | EGFR T790M ASO 1 target sequence | TCATCATGCAGCTCATGCCC |
| 34 | EGFR T790M ASO 2 target sequence | CATCATGCAGCTCATGCCCT |
| 35 | EGFR T790M ASO 3 target sequence | ATCATGCAGCTCATGCCCTT |
| 36 | EGFR T790M ASO 4 target sequence | TCATGCAGCTCATGCCCTTC |
| 37 | KRAS G12D ASO 1 modified sequence target sequence | GTGGTAGTTGGAGCTGATGG |
| 38 | KRAS G12D ASO 2 modified sequence target sequence | TGGTAGTTGGAGCTGATGGC |
| 39 | KRAS G12D ASO 3 modified sequence target sequence | GAGCTGATGGCGTAGGCAAG |
| 40 | KRAS G12D ASO 4 modified sequence target sequence | AGCTGATGGCGTAGGCAAGA |
| 41 | KRAS G12D ASO 5 modified sequence target sequence | GCTGATGGCGTAGGCAAGAG |
| 42 | KRAS G12V ASO 1 modified sequence target sequence | TGTGGTAGTTGGAGCTGTTG |
| 43 | KRAS G12V ASO 2 modified sequence target sequence | GTGGTAGTTGGAGCTGTTGG |
| 44 | KRAS G12V ASO 3 modified sequence target sequence | CTGTTGGCGTAGGCAAGAGT |
| 45 | EGFR L858R ASO 1 modified sequence target sequence | GATCACAGATTTTGGGCGGC |
| 46 | EGFR L858R ASO 2 modified sequence target sequence | ATCACAGATTTTGGGCGGCC |
| 47 | EGFR L858R ASO 3 modified sequence target sequence | TCACAGATTTTGGGCGGCCA |
| 48 | EGFR L858R ASO 4 modified sequence target sequence | CACAGATTTTGGGCGGCCAA |
| 49 | EGFR T790M ASO 1 modified sequence target sequence | TCATCATGCAGCTCATGCCC |
| 50 | EGFR T790M ASO 2 modified sequence target sequence | CATCATGCAGCTCATGCCCT |
| 51 | EGFR T790M ASO 3 modified sequence target sequence | ATCATGCAGCTCATGCCCTT |
| 52 | EGFR T790M ASO 4 modified sequence target sequence | TCATGCAGCTCATGCCCTTC |
| 53 | Anti-miR-125b ASO (125b-ASO) sequence | UCACAAGUUAGGGUCUCAGGGA |
| 54 | negative control RNA (NC RNA) sequence | CAGUACUUUUGUGUAGUACAA |
| 55 | FAM-labeled NC-ASO sequence | FAMCAGUACUUUUGUGUAGUACAA |
| 56 | 3p-125b-ASOs DNA template sequence | Sense: GTAATACGACTCACTATAGGAAGTTAGGGTCTCAGGCT |
| 57 | 3p-125b-ASOs DNA template sequence | Antisense: AGCCTGAGACCCTAACTTCCTATAGTGAGTCGTATTAC |
| 58 | 3′ adapter sequence | TGGAATTCTCGGGTGCCAAGG |
| 129 | KRAS G12D ASO 1 modified sequence | +C*mC*mA*+T*mC*A*G*C*T*C*C*A*A*C*T*mA*mC*mC*mA*+C |
| 130 | KRAS G12D ASO 2 modified sequence | +G*mC*mC*mA*+T*C*A*G*C*T*C*C*A*A*C*mT*mA*mC*mC*+A |
| 131 | KRAS G12D ASO 3 modified sequence | +C*mT*mT*mG*mC*C*T*A*C*G*C*C*A*+T*C*mA*mG*mC*mT*+C |
| 132 | KRAS G12D ASO 4 modified sequence | +T*mC*mT*mT*mG*C*C*T*A*C*G*C*C*A*+T*mC*mA*mG*mC*+T |
| 133 | KRAS G12D ASO 5 modified sequence | +C*mT*mC*mT*mT*G*C*C*T*A*C*G*C*C*A*+T*mC*mA*mG*+C |
| 134 | KRAS G12V ASO 1 modified sequence | +C*mA*+A*mC*mA*G*C*T*C*C*A*A*C*T*A*mC*mC*mA*mC*+A |
| 135 | KRAS G12V ASO 2 modified sequence | +C*mC*mA*+A*mC*A*G*C*T*C*C*A*A*C*T*mA*mC*mC*mA*+C |
| 136 | KRAS G12V ASO 3 modified sequence | +A*mC*mT*mC*mT*T*G*C*C*T*A*C*G*C*C*mA*+A*mC*mA*+G |
| 137 | EGFR L858R ASO 1 modified sequence | +G*mC*+C*mG*C*C*C*A*A*A*A*T*C*T*G*mT*mG*mA*mT*+C |
| 138 | EGFR L858R ASO 2 modified sequence | +G*mG*mC*+C*mG*C*C*C*A*A*A*A*T*C*T*mG*mT*mG*mA*+T |
| 139 | EGFR L858R ASO 3 modified sequence | +T*mG*mG*mC*+C*G*C*C*C*A*A*A*A*T*C*mT*mG*mT*mG*+A |
| 140 | EGFR L858R ASO 4 modified sequence | +T*mT*mG*mG*mC*+C*G*C*C*C*A*A*A*A*T*mC*mT*mG*mT*+G |
| 141 | EGFR T790M ASO 1 modified sequence | +G*mG*mG*mC*mA*T*G*A*G*C*T*G*C*+A*T*mG*mA*mT*mG*+A |
| 142 | EGFR T790M ASO 2 modified sequence | +A*mG*mG*mG*mC*A*T*G*A*G*C*T*G*C*+A*mT*mG*mA*mT*+G |
| 143 | EGFR T790M ASO 3 modified sequence | +A*mA*mG*mG*mG*C*A*T*G*A*G*C*T*G*C*+A*mT*mG*mA*+T |
| 144 | EGFR T790M ASO 4 modified sequence | +G*mA*mA*mG*mG*G*C*A*T*G*A*G*C*T*G*mC*+A*mT*mG*+A |

### REFERENCES

Ho, V., Yong, H. Y., Chevrier, M., Narang, V., Lum, J., Toh, Y.-X., Lee, B., Chen, J., Tan, E. Y., Luo, D., & Fink, K. (2019). RIG-I activation by a designer short RNA ligand protects human immune cells against dengue virus infection without causing cytotoxicity. Journal of Virology 93, e00102-e00119.
Kasinski, A. L., & Slack, F. J. (2013). Generation of mouse lung epithelial cells. Bio Protocol 3, e837.
Luo, D., Kohlway, A., Vela, A., & Pyle, A. M. (2012). Visualizing the determinants of viral RNA recognition by innate immune sensor RIG-I. Structure 20, 1983-1988.
Pham, C. T., Jayasinghe, M. K., Pham, T. T., Yan, Y., Wei, L., Usman, W. M., Chen, H., Pirisinu, M., Gong, J., Kim, S., Peng, B., Wang, W., Chan, C., Ma, V., Nguyen, N. T. H., Kappi, D., Nguyen, X.-H., Cho, W. C., Shi, J., & Le, M. T. N. (2021). Covalent conjugation of extracellular vesicles with peptides with nanobodies for targeted therapeutic delivery. Journal of Extracellular Vesicles 10, e12057.
Usman, W. M., Pham, T. C., Kwok, Y. Y., Vu, L. T., Ma, V., Peng, B., Chan, Y. S., Wei, L., Chin, S. M., Azad, A., He, A. B.-L., Leung, A. Y. H., Yang, M., Shyh-Chang, N., Cho, W. C., Shi, J., & Le, M. T. N. (2018). Efficient RNA drug delivery using red blood cell extracellular vesicles. Nature Communications 9, 2359.
Yong, H. Y., Zheng, J., Ho, V. C. Y., Nguyen, M. T., Fink, K., Griffin, P. R., & Luo, D. (2019). Structure-guided design of immunomodulatory RNAs specifically targeting the cytoplasmic viral RNA sensor RIG-I. FEBS Letters 593, 3003-3014.

1. A composition comprising a vesicle for delivery of a retinoic acid inducible gene I receptor (RIG-I) agonist to a cell.

2. The composition of claim 1, wherein the RIG-I agonist is selected from a group consisting of an immunomodulatory RNA (immRNA), an antisense oligonucleotide (ASO), a small interfering RNA (siRNA), and combinations thereof.

3. The composition of claim 2, wherein the RIG-I agonist comprises an immunomodulatory RNA (immRNA) and/or an antisense oligonucleotide (ASO).

4. The composition of claim 2, wherein the immunomodulatory RNA (immRNA) comprises a sequence 5′-GGAUUUCCACCUUCGGGGGAAAUCC-3′ (SEQ ID NO: 1).

5. The composition of claim 2, wherein the immunomodulatory RNA (immRNA) further comprises a modification in the 5′ and/or 3′ end, optionally wherein the modification is selected from a group consisting of a 5′ triphosphate cap, phosphorylation, methylation, adenylation, and combinations thereof.

6. The composition of claim 2, wherein the antisense oligonucleotide (ASO) comprises a miRNA-125b-ASO, a KRAS-ASO, or an EGFR targeting ASO.

7. The composition of claim 6, wherein the miRNA-125b-ASO comprises a sequence 5′-GGAAGUUAGGGUCUCAGGCCCUAACUUCC-3′ (SEQ ID NO: 2).

8. The composition of claim 6, wherein the KRAS-ASO is selected from a group consisting of a KRAS-G12D ASO, KRAS-G12V ASO, a KRAS-G12C ASO, and a KRAS-G12S ASO, optionally wherein the KRAS-G12D ASO comprises any one of the sequences below

| SEQ | | | SEQ | |
|---|---|---|---|---|
| ID | | ASO sequence | ID | Target sequence |
| No. | Name | (5′ -→ 3′) | No. | (5′ → 3′) |
| 3 | KRAS G12D ASO 1 | CCATCAGCTCCAACTACCAC | 21 | GTGGTAGTTGGAGCTGATGG |
| 4 | KRAS G12D ASO 2 | GCCATCAGCTCCAACTACCA | 22 | TGGTAGTTGGAGCTGATGGC |
| 5 | KRAS G12D ASO 3 | CTTGCCTACGCCATCAGCTC | 23 | GAGCTGATGGCGTAGGCAAG |
| 6 | KRAS G12D ASO 4 | TCTTGCCTACGCCATCAGCT | 24 | AGCTGATGGCGTAGGCAAGA |
| 7 | KRAS G12D ASO 5 | CTCTTGCCTACGCCATCAGC | 25 | GCTGATGGCGTAGGCAAGAG |

or wherein the KRAS-G12V ASO comprises any one of the sequences below

| SEQ | | | SEQ | |
|---|---|---|---|---|
| ID | | ASO sequence | ID | Target sequence |
| No. | Name | (5′ → 3′) | No. | (5′ → 3′) |
| 8 | KRAS G12V ASO 1 | CAACAGCTCCAACTACCACA | 26 | TGTGGTAGTTGGAGCTGTTG |
| 9 | KRAS G12V ASO 2 | CCAACAGCTCCAACTACCAC | 27 | GTGGTAGTTGGAGCTGTTGG |
| 10 | KRAS G12V ASO 3 | ACTCTTGCCTACGCCAACAG | 28 | CTGTTGGCGTAGGCAAGAGT |

9. The composition of claim 6, wherein the EGFR targeting ASO is selected from a group consisting of a EGFR L858R targeting ASO, or a EGFR T790M targeting ASO, optionally wherein the EGFR L858R targeting ASO comprises any one of the sequences below,

| SEQ | | | SEQ | |
|---|---|---|---|---|
| ID | | ASO sequence | ID | Target sequence |
| No. | Name | (5′ -→ 3′) | No. | (5′ → 3′) |
| 11 | EGFR L858R | GCCGCCCAAAATCTGTGATC | 29 | GATCACAGATTTTGGGCGGC |
| | ASO 1 | | | |
| 13 | EGFR L858R | GGCCGCCCAAAATCTGTGAT | 30 | ATCACAGATTTTGGGCGGCC |
| | ASO 2 | | | |
| 13 | EGFR L858R | TGGCCGCCCAAAATCTGTGA | 31 | TCACAGATTTTGGGCGGCCA |
| | ASO 3 | | | |
| 14 | EGFR L858R | TTGGCCGCCCAAAATCTGTG | 32 | CACAGATTTTGGGCGGCCAA |
| | ASO 4 | | | |

or wherein the EGFR T790M targeting ASO comprises any one of the sequences below.

| SEQ | | | SEQ | |
|---|---|---|---|---|
| ID | | ASO sequence | ID | Target sequence |
| No. | Name | (5′ -→ 3′) | No. | (5′ → 3′) |
| 15 | EGFR T790M ASO 1 | GGGCATGAGCTGCATGATGA | 33 | TCATCATGCAGCTCATGCCC |
| 16 | EGFR T790M ASO 2 | AGGGCATGAGCTGCATGATG | 34 | CATCATGCAGCTCATGCCCT |
| 17 | EGFR T790M ASO 3 | AAGGGCATGAGCTGCATGAT | 35 | ATCATGCAGCTCATGCCCTT |
| 18 | EGFR T790M ASO 4 | GAAGGGCATGAGCTGCATGA | 36 | TCATGCAGCTCATGCCCTTC |

10. The composition of claim 6, wherein the antisense oligonucleotide (ASO) further comprises a modification in the 5′ and/or 3′ end.

11. The composition of claim 10, wherein the modification is selected from a group consisting of a 5′ triphosphate cap, phosphorylation, methylation, adenylation, locked nucleic acid, phosphorothioate, 2-methoxyethyl, and combinations thereof.

12. The composition of claim 1, wherein the vesicle for delivery of a retinoic acid inducible gene I receptor (RIG-I) agonist to a cell comprises an extracellular vesicle or a lipid nanoparticle.

13. The composition of claim 12, wherein the extracellular vesicle is selected from a group consisting of a red blood cell-derived extracellular vesicle (RBCEV), a milk derived EV, a plasmid derived EV, and a cancer cell derived EV.

14. The composition of claim 13, wherein the extracellular vesicle is further conjugated to an antibody, optionally wherein the antibody is selected from a group consisting of a nanobody, a monoclonal antibody, and a single-chain antibody.

15. The composition of claim 14, wherein the antibody is a nanobody that binds to a cancer marker, optionally wherein the cancer marker is selected from a group consisting of epidermal growth factor receptor (EGFR), HER1, HER2, HER3, human growth factor (HGF), CXCR4, PD-L1, CD49a, CD49b, CD49c, CD49d, CD49e, CD49f, ITGA7, ITGA8, ITGA9, ITGA10, ITGA11, CD11D, CD103, CD11a, CD11b, CD51, CD41, CD11c, CD29, CD18, CD61, CD104, ITGB5, ITGB6, ITGB, ITGB8, FOLR1, FOLR2, or FOLR3.

16. A composition comprising:
a red blood cell-derived extracellular vesicle (RBCEV);
an immunomodulatory RNA (immRNA) comprising or consisting of 5′-GGAUUUCCACCUUCGGGGGAAAUCC-3′ (SEQ ID NO: 1), wherein the immunomodulatory RNA (immRNA) further comprises a 5′ triphosphate cap; and/or
an antisense oligonucleotide (ASO) comprising or consisting of 5′-GGAAGUUAGGGUCUCAGGCCCUAACUUCC-3′ (SEQ ID NO: 2), wherein the antisense oligonucleotide (ASO) further comprises a 5′ triphosphate cap.

17. A composition comprising:
a lipid nanoparticle;
an immunomodulatory RNA (immRNA) comprising 5′-GGAUUUCCACCUUCGGGGGAAAUCC-3′ (SEQ ID NO: 1), wherein the immunomodulatory RNA (immRNA) further comprises a 5′ triphosphate cap; and/or
a KRAS-ASO.

18-24. (canceled)


SEQUENCE LISTING (144 sequences)

SEQ ID NO: 1 - RNA, 25 nt, synthetic construct
  source 1..25: mol_type = other RNA
ggatttccaccttcgggggaaatcc

SEQ ID NO: 2 - RNA, 29 nt, synthetic construct
  source 1..29: mol_type = other RNA
ggaagttagggtctcaggccctaacttcc

SEQ ID NO: 3 - DNA, 20 bp, synthetic construct
  source 1..20: mol_type = other DNA
ccatcagctccaactaccac

SEQ ID NO: 4 - DNA, 20 bp, synthetic construct
  source 1..20: mol_type = other DNA
gccatcagctccaactacca

SEQ ID NO: 5 - DNA, 20 bp, synthetic construct
  source 1..20: mol_type = other DNA
cttgcctacgccatcagctc

SEQ ID NO: 6 - DNA, 20 bp, synthetic construct
  source 1..20: mol_type = other DNA
tcttgcctacgccatcagct

SEQ ID NO: 7 - DNA, 20 bp, synthetic construct
  source 1..20: mol_type = other DNA
ctcttgcctacgccatcagc

SEQ ID NO: 8 - DNA, 20 bp, synthetic construct
  source 1..20: mol_type = other DNA
caacagctccaactaccaca

SEQ ID NO: 9 - DNA, 20 bp, synthetic construct
  source 1..20: mol_type = other DNA
ccaacagctccaactaccac

SEQ ID NO: 10 - DNA, 20 bp, synthetic construct
  source 1..20: mol_type = other DNA
actcttgcctacgccaacag

SEQ ID NO: 11 - DNA, 20 bp, synthetic construct
  source 1..20: mol_type = other DNA
gccgcccaaaatctgtgatc

SEQ ID NO: 12 - DNA, 20 bp, synthetic construct
  source 1..20: mol_type = other DNA
ggccgcccaaaatctgtgat

SEQ ID NO: 13 - DNA, 20 bp, synthetic construct
  source 1..20: mol_type = other DNA
tggccgcccaaaatctgtga

SEQ ID NO: 14 - DNA, 20 bp, synthetic construct
  source 1..20: mol_type = other DNA
ttggccgcccaaaatctgtg

SEQ ID NO: 15 - DNA, 20 bp, synthetic construct
  source 1..20: mol_type = other DNA
gggcatgagctgcatgatga

SEQ ID NO: 16 - DNA, 20 bp, synthetic construct
  source 1..20: mol_type = other DNA
agggcatgagctgcatgatg

SEQ ID NO: 17 - DNA, 20 bp, synthetic construct
  source 1..20: mol_type = other DNA
aagggcatgagctgcatgat

SEQ ID NO: 18 - DNA, 20 bp, synthetic construct
  source 1..20: mol_type = other DNA
gaagggcatgagctgcatga

SEQ ID NO: 19 - RNA, 25 nt, synthetic construct
  source 1..25: mol_type = other RNA
  modified_base 1: mod_base = OTHER; note = triphosphate
ggatttccaccttcgggggaaatcc

SEQ ID NO: 20 - DNA, 29 bp, synthetic construct
  source 1..29: mol_type = other DNA
  modified_base 1: mod_base = OTHER; note = triphosphate
ggaagttagggtctcaggccctaacttcc

SEQ ID NO: 21 - RNA, 20 nt, Homo sapiens
  source 1..20: mol_type = mRNA
gtggtagttggagctgatgg

SEQ ID NO: 22 - RNA, 20 nt, Homo sapiens
  source 1..20: mol_type = mRNA
tggtagttggagctgatggc

SEQ ID NO: 23 - RNA, 20 nt, Homo sapiens
  source 1..20: mol_type = mRNA
gagctgatggcgtaggcaag

SEQ ID NO: 24 - RNA, 20 nt, Homo sapiens
  source 1..20: mol_type = mRNA
agctgatggcgtaggcaaga

SEQ ID NO: 25 - RNA, 20 nt, Homo sapiens
  source 1..20: mol_type = mRNA
gctgatggcgtaggcaagag

SEQ ID NO: 26 - RNA, 20 nt, Homo sapiens
  source 1..20: mol_type = mRNA
tgtggtagttggagctgttg

SEQ ID NO: 27 - RNA, 20 nt, Homo sapiens
  source 1..20: mol_type = mRNA
gtggtagttggagctgttgg

SEQ ID NO: 28 - RNA, 20 nt, Homo sapiens
  source 1..20: mol_type = mRNA
ctgttggcgtaggcaagagt

SEQ ID NO: 29 - RNA, 20 nt, Homo sapiens
  source 1..20: mol_type = mRNA
gatcacagattttgggcggc

SEQ ID NO: 30 - RNA, 20 nt, Homo sapiens
  source 1..20: mol_type = mRNA
atcacagattttgggcggcc

SEQ ID NO: 31 - RNA, 20 nt, Homo sapiens
  source 1..20: mol_type = mRNA
tcacagattttgggcggcca

SEQ ID NO: 32 - RNA, 20 nt, Homo sapiens
  source 1..20: mol_type = mRNA
cacagattttgggcggccaa

SEQ ID NO: 33 - RNA, 20 nt, Homo sapiens
  source 1..20: mol_type = mRNA
tcatcatgcagctcatgccc

SEQ ID NO: 34 - RNA, 20 nt, Homo sapiens
  source 1..20: mol_type = mRNA
catcatgcagctcatgccct

SEQ ID NO: 35 - RNA, 20 nt, Homo sapiens
  source 1..20: mol_type = mRNA
atcatgcagctcatgccctt

SEQ ID NO: 36 - RNA, 20 nt, Homo sapiens
  source 1..20: mol_type = mRNA
tcatgcagctcatgcccttc

SEQ ID NO: 37 - RNA, 20 nt, Homo sapiens
  source 1..20: mol_type = mRNA
gtggtagttggagctgatgg

SEQ ID NO: 38 - RNA, 20 nt, Homo sapiens
  source 1..20: mol_type = mRNA
tggtagttggagctgatggc

SEQ ID NO: 39 - RNA, 20 nt, Homo sapiens
  source 1..20: mol_type = mRNA
gagctgatggcgtaggcaag

SEQ ID NO: 40 - RNA, 20 nt, Homo sapiens
  source 1..20: mol_type = mRNA
agctgatggcgtaggcaaga

SEQ ID NO: 41 - RNA, 20 nt, Homo sapiens
  source 1..20: mol_type = mRNA
gctgatggcgtaggcaagag

SEQ ID NO: 42 - RNA, 20 nt, Homo sapiens
  source 1..20: mol_type = mRNA
tgtggtagttggagctgttg

SEQ ID NO: 43 - RNA, 20 nt, Homo sapiens
  source 1..20: mol_type = mRNA
gtggtagttggagctgttgg

SEQ ID NO: 44 - RNA, 20 nt, Homo sapiens
  source 1..20: mol_type = mRNA
ctgttggcgtaggcaagagt

SEQ ID NO: 45 - RNA, 20 nt, Homo sapiens
  source 1..20: mol_type = mRNA
gatcacagattttgggcggc

SEQ ID NO: 46 - RNA, 20 nt, Homo sapiens
  source 1..20: mol_type = mRNA
atcacagattttgggcggcc

SEQ ID NO: 47 - RNA, 20 nt, Homo sapiens
  source 1..20: mol_type = mRNA
tcacagattttgggcggcca

SEQ ID NO: 48 - RNA, 20 nt, Homo sapiens
  source 1..20: mol_type = mRNA
cacagattttgggcggccaa

SEQ ID NO: 49 - RNA, 20 nt, Homo sapiens
  source 1..20: mol_type = mRNA
tcatcatgcagctcatgccc

SEQ ID NO: 50 - RNA, 20 nt, Homo sapiens
  source 1..20: mol_type = mRNA
catcatgcagctcatgccct

SEQ ID NO: 51 - RNA, 20 nt, Homo sapiens
  source 1..20: mol_type = mRNA
atcatgcagctcatgccctt

SEQ ID NO: 52 - RNA, 20 nt, Homo sapiens
  source 1..20: mol_type = mRNA
tcatgcagctcatgcccttc

SEQ ID NO: 53 - RNA, 22 nt, synthetic construct
  source 1..22: mol_type = other RNA
tcacaagttagggtctcaggga

SEQ ID NO: 54 - RNA, 21 nt, synthetic construct
  source 1..21: mol_type = other RNA
cagtacttttgtgtagtacaa

SEQ ID NO: 55 - RNA, 21 nt, synthetic construct
  source 1..21: mol_type = other RNA
  modified_base 1: mod_base = OTHER; note = fluorescein
cagtacttttgtgtagtacaa

SEQ ID NO: 56 - DNA, 38 bp, Homo sapiens
  source 1..38: mol_type = genomic DNA
gtaatacgactcactataggaagttagggtctcaggct

SEQ ID NO: 57 - DNA, 38 bp, Homo sapiens
  source 1..38: mol_type = genomic DNA
agcctgagaccctaacttcctatagtgagtcgtattac

SEQ ID NO: 58 - DNA, 21 bp, synthetic construct
  source 1..21: mol_type = other DNA
tggaattctcgggtgccaagg

SEQ ID NO: 59 - DNA, 21 bp, synthetic construct
  source 1..21: mol_type = other DNA
aggtcggtgtgaacggatttg

SEQ ID NO: 60 - DNA, 23 bp, synthetic construct
  source 1..23: mol_type = other DNA
tgtagaccatgtagttgaggtca

SEQ ID NO: 61 - DNA, 21 bp, synthetic construct
  source 1..21: mol_type = other DNA
ggagcgagatccctccaaaat

SEQ ID NO: 62 - DNA, 23 bp, synthetic construct
  source 1..23: mol_type = other DNA
ggctgttgtcatacttctcatgg

SEQ ID NO: 63 - DNA, 19 bp, synthetic construct
  source 1..19: mol_type = other DNA
gagagtcacgggacccact

SEQ ID NO: 64 - DNA, 20 bp, synthetic construct
  source 1..20: mol_type = other DNA
cggtcttagcatctccaacg

SEQ ID NO: 65 - DNA, 22 bp, synthetic construct
  source 1..22: mol_type = other DNA
gccattacactgtgcttggaga

SEQ ID NO: 66 - DNA, 22 bp, synthetic construct
  source 1..22: mol_type = other DNA
ccagttgcaatatcctccacca

SEQ ID NO: 67 - DNA, 24 bp, synthetic construct
  source 1..24: mol_type = other DNA
tgatgcactattccaagaactaac

SEQ ID NO: 68 - DNA, 22 bp, synthetic construct
  source 1..22: mol_type = other DNA
tctgtgagacgagttagccaag

SEQ ID NO: 69 - DNA, 22 bp, synthetic construct
  source 1..22: mol_type = other DNA
gagcaacttctttcaaccacag

SEQ ID NO: 70 - DNA, 21 bp, synthetic construct
  source 1..21: mol_type = other DNA
cacttccttctgccaaacttg

SEQ ID NO: 71 - DNA, 20 bp, synthetic construct
  source 1..20: mol_type = other DNA
acagccaagacatccttcgt

SEQ ID NO: 72 - DNA, 23 bp, synthetic construct
  source 1..23: mol_type = other DNA
aaaagttgatcttctccaaacca

SEQ ID NO: 73 - DNA, 23 bp, synthetic construct
  source 1..23: mol_type = other DNA
cacaaagaagtgtcctgcttggt

SEQ ID NO: 74 - DNA, 23 bp, synthetic construct
  source 1..23: mol_type = other DNA
aagcgcatatattcatccagaat

SEQ ID NO: 75 - DNA, 20 bp, synthetic construct
  source 1..20: mol_type = other DNA
ctgcctcacagctagtgacc

SEQ ID NO: 76 - DNA, 19 bp, synthetic construct
  source 1..19: mol_type = other DNA
ccggcgctggagattattg

SEQ ID NO: 77 - DNA, 20 bp, synthetic construct
  source 1..20: mol_type = other DNA
aggagacagatggagacaca

SEQ ID NO: 78 - DNA, 18 bp, synthetic construct
  source 1..18: mol_type = other DNA
cagaactgggcagtaccc

SEQ ID NO: 79 - DNA, 24 bp, synthetic construct
  source 1..24: mol_type = other DNA
aacctcacctacagggcggacttc

SEQ ID NO: 80 - DNA, 25 bp, synthetic construct
  source 1..25: mol_type = other DNA
tcccacgtcaatctttcctcttgct

SEQ ID NO: 81 - DNA, 20 bp, synthetic construct
  source 1..20: mol_type = other DNA
ctctcctgttgtgcttctcc

SEQ ID NO: 82 - DNA, 22 bp, synthetic construct
  source 1..22: mol_type = other DNA
gtcaaagttcatcctgtccttg

SEQ ID NO: 83 - DNA, 20 bp, synthetic construct
  source 1..20: mol_type = other DNA
cggaaagaagtgttgcggtt

SEQ ID NO: 84 - DNA, 20 bp, synthetic construct
  source 1..20: mol_type = other DNA
ttttcctgggagtgaggcag

SEQ ID NO: 85 - DNA, 19 bp, synthetic construct
  source 1..19: mol_type = other DNA
accagccgtggaccaagag

SEQ ID NO: 86 - DNA, 19 bp, synthetic construct
  source 1..19: mol_type = other DNA
taccaaggccctgaggcac

SEQ ID NO: 87 - DNA, 18 bp, synthetic construct
  source 1..18: mol_type = other DNA
agggcgttttatcttgcg

SEQ ID NO: 88 - DNA, 20 bp, synthetic construct
  source 1..20: mol_type = other DNA
tggagcccagcattttctct

SEQ ID NO: 89 - DNA, 20 bp, synthetic construct
  source 1..20: mol_type = other DNA
tggtcctggtgaagctggaa

SEQ ID NO: 90 - DNA, 23 bp, synthetic construct
  source 1..23: mol_type = other DNA
gatgtcgtcatagaggctgttgg

SEQ ID NO: 91 - DNA, 20 bp, synthetic construct
  source 1..20: mol_type = other DNA
gtgtgccacctgtgccatcc

SEQ ID NO: 92 - DNA, 20 bp, synthetic construct
  source 1..20: mol_type = other DNA
gccaccaccagcagcaagag

SEQ ID NO: 93 - DNA, 22 bp, synthetic construct
  source 1..22: mol_type = other DNA
tacaggctggagtgtgctgaga

SEQ ID NO: 94 - DNA, 22 bp, synthetic construct
  source 1..22: mol_type = other DNA
ctccactttcagagccttcgca

SEQ ID NO: 95 - DNA, 22 bp, synthetic construct
  source 1..22: mol_type = other DNA
tagccaacatgtcctcacagac

SEQ ID NO: 96 - DNA, 22 bp, synthetic construct
  source 1..22: mol_type = other DNA
tcttctaccactggtttcatgc

SEQ ID NO: 97 - DNA, 22 bp, synthetic construct
  source 1..22: mol_type = other DNA
atcatcggcattttgaacgagg

SEQ ID NO: 98 - DNA, 20 bp, synthetic construct
  source 1..20: mol_type = other DNA
gcagctccatgagaacacta

SEQ ID NO: 99 - DNA, 23 bp, synthetic construct
  source 1..23: mol_type = other DNA
ctctgttgacaagcaatgagacg

SEQ ID NO: 100 - DNA, 22 bp, synthetic construct
  source 1..22: mol_type = other DNA
tcttcagtatgtctagcccctg

SEQ ID NO: 101 - DNA, 19 bp, synthetic construct
  source 1..19: mol_type = other DNA
gccacggcacagtcattga

SEQ ID NO: 102 - DNA, 21 bp, synthetic construct
  source 1..21: mol_type = other DNA
tgctgatggcctgattgtctt

SEQ ID NO: 103 - DNA, 19 bp, synthetic construct
  source 1..19: mol_type = other DNA
gtgctccttgtcaacagcg

SEQ ID NO: 104 - DNA, 21 bp, synthetic construct
  source 1..21: mol_type = other DNA
ggggagtttcaggttcctgta

SEQ ID NO: 105 - DNA, 23 bp, synthetic construct
  source 1..23: mol_type = other DNA
cttcaatacgtcagacattcggg

SEQ ID NO: 106 - DNA, 22 bp, synthetic construct
  source 1..22: mol_type = other DNA
gtaacgccaggaattgttgcta

SEQ ID NO: 107 - DNA, 22 bp, synthetic construct
  source 1..22: mol_type = other DNA
gcaactgttcctgaactcaact

SEQ ID NO: 108 - DNA, 21 bp, synthetic construct
  source 1..21: mol_type = other DNA
atcttttggggtccgtcaact

SEQ ID NO: 109 - DNA, 22 bp, synthetic construct
  source 1..22: mol_type = other DNA
gactcttgcgtcaacttcaagg

SEQ ID NO: 110 - DNA, 21 bp, synthetic construct
  source 1..21: mol_type = other DNA
caggctgtcttttgtcaacga

SEQ ID NO: 111 - DNA, 23 bp, synthetic construct
  source 1..23: mol_type = other DNA
agccttatcggaaatgatccagt

SEQ ID NO: 112 - DNA, 20 bp, synthetic construct
  source 1..20: mol_type = other DNA
ggccttgtagacaccttggt

SEQ ID NO: 113 - DNA, 23 bp, synthetic construct
  source 1..23: mol_type = other DNA
tctataccacttcacaagtcgga

SEQ ID NO: 114 - DNA, 23 bp, synthetic construct
  source 1..23: mol_type = other DNA
tctataccacttcacaagtcgga

SEQ ID NO: 115 - DNA, 21 bp, synthetic construct
  source 1..21: mol_type = other DNA
tggtttgccatcgttttgctg

SEQ ID NO: 116 - DNA, 22 bp, synthetic construct
  source 1..22: mol_type = other DNA
acaggtgaggttcactgtttct

SEQ ID NO: 117 - DNA, 19 bp, synthetic construct
  source 1..19: mol_type = other DNA
cctgtagcccacgtcgtag

SEQ ID NO: 118 - DNA, 22 bp, synthetic construct
  source 1..22: mol_type = other DNA
gggagtagacaaggtacaaccc

SEQ ID NO: 119 - DNA, 24 bp, synthetic construct
  source 1..24: mol_type = other DNA
ctggtaatgatgagctactactgg

SEQ ID NO: 120 - DNA, 21 bp, synthetic construct
  source 1..21: mol_type = other DNA
ccttctccaaggggaatccaa

SEQ ID NO: 121 - DNA, 20 bp, synthetic construct
  source 1..20: mol_type = other DNA
ggtcctggcacaaatgagga

SEQ ID NO: 122 - DNA, 20 bp, synthetic construct
  source 1..20: mol_type = other DNA
tccaagcagcagatgagtcc

SEQ ID NO: 123 - DNA, 23 bp, synthetic construct
  source 1..23: mol_type = other DNA
aagactgagtgagaaggagtgag

SEQ ID NO: 124 - DNA, 23 bp, synthetic construct
  source 1..23: mol_type = other DNA
gagatgccagaatttgagcagtg

SEQ ID NO: 125 - DNA, 43 bp, synthetic construct
  source 1..43: mol_type = other DNA
ggatttcccccgaaggtggaaatcctatagtgagtcgtattac

SEQ ID NO: 126 - DNA, 43 bp, synthetic construct
  source 1..43: mol_type = other DNA
gtaatacgactcactataggatttccaccttcgggggaaatcc

SEQ ID NO: 127 - DNA, 38 bp, synthetic construct; identical to SEQ ID NO: 57
  source 1..38: mol_type = other DNA

SEQ ID NO: 128 - DNA, 38 bp, synthetic construct; identical to SEQ ID NO: 56
  source 1..38: mol_type = other DNA

SEQ ID NO: 129-144 - skipped (16 entries)